SEQUENCE LISTING (131 sequences)

SEQ ID NO: 1 - DNA, 16 bp, Artificial Sequence
  Synthetic polynucleotide
tgaaggccacactaac

SEQ ID NO: 2 - DNA, 16 bp, Artificial Sequence
  Synthetic polynucleotide
atgaaggccacactaa

SEQ ID NO: 3 - DNA, 15 bp, Artificial Sequence
  Synthetic polynucleotide
gaaggccacactaac

SEQ ID NO: 4 - DNA, 16 bp, Artificial Sequence
  Synthetic polynucleotide
cacggtgctgaaagag

SEQ ID NO: 5 - DNA, 16 bp, Artificial Sequence
  Synthetic polynucleotide
acggtgctgaaagaga

SEQ ID NO: 6 - DNA, 15 bp, Artificial Sequence
  Synthetic polynucleotide
cggtgctgaaagaga

SEQ ID NO: 7 - DNA, 16 bp, Artificial Sequence
  Synthetic polynucleotide
tccttgaatggttgca

SEQ ID NO: 8 - DNA, 16 bp, Artificial Sequence
  Synthetic polynucleotide
ttgaatggttgcacgg

SEQ ID NO: 9 - DNA, 16 bp, Artificial Sequence
  Synthetic polynucleotide
cggtgctgaaagagag

SEQ ID NO: 10 - DNA, 16 bp, Artificial Sequence
  Synthetic polynucleotide
atttggcagtgggaag

SEQ ID NO: 11 - DNA, 16 bp, Artificial Sequence
  Synthetic polynucleotide
tttggcagtgggaagc

SEQ ID NO: 12 - DNA, 16 bp, Artificial Sequence
  Synthetic polynucleotide
ttggcagtgggaagca

SEQ ID NO: 13 - DNA, 16 bp, Artificial Sequence
  Synthetic polynucleotide
tcaaaaacataacgcc

SEQ ID NO: 14 - DNA, 16 bp, Artificial Sequence
  Synthetic polynucleotide
gtcaaaaacataacgc

SEQ ID NO: 15 - DNA, 17 bp, Artificial Sequence
  Synthetic polynucleotide
ggtcaaaaacataacgc

SEQ ID NO: 16 - DNA, 16 bp, Artificial Sequence
  Synthetic polynucleotide
ggtcaaaaacataacg

SEQ ID NO: 17 - DNA, 16 bp, Artificial Sequence
  Synthetic polynucleotide
aggtcaaaaacataac

SEQ ID NO: 18 - DNA, 17 bp, Artificial Sequence
  Synthetic polynucleotide
aggtcaaaaacataacg

SEQ ID NO: 19 - DNA, 15 bp, Artificial Sequence
  Synthetic polynucleotide
taggtcaaaaacata

SEQ ID NO: 20 - DNA, 15 bp, Artificial Sequence
  Synthetic polynucleotide
caaaaacataacgcc

SEQ ID NO: 21 - DNA, 16 bp, Artificial Sequence
  Synthetic polynucleotide
gatttggcattggaag

SEQ ID NO: 22 - DNA, 16 bp, Artificial Sequence
  Synthetic polynucleotide
ggaaggccatgtcaac

SEQ ID NO: 23 - DNA, 16 bp, Artificial Sequence
  Synthetic polynucleotide
ggcattgatgggggaa

SEQ ID NO: 24 - DNA, 17 bp, Artificial Sequence
  Synthetic polynucleotide
tcagaaatcataacgcc

SEQ ID NO: 25 - DNA, 287 bp, Mus musculus
atttttctgagttagtgtggccttcatctggtaatgtactacctgaggggggaggtgccgcctctctttcagcaccgtgcaaccattcaaggagggtgtgttgttcaccacatctgcttcccactgccaaatcaggcctcagaaaagctttctggaagtgacgccagcttcagggacaaggcccaagtttctaggggtcaacaccgttccatggttcctgaagagatggtagactatggaacgtaggcgttatgtttttgacctatgtaacatggtccactaactct

SEQ ID NO: 26 - DNA, 291 bp, Homo sapiens
agtctttccaagttgacatggccttcctggaggaattaccacttagggtagaggcaccccttcccccatcaatgccactgccccacattggaggaggggttgtttatgttcaccatgtgcctgcttccaatgccaaatccagcctcagaaagctttctggaagtgacgccaacttcaggggcaaggccctggttctggggtcagcaccattccgtggttcctgaagagatggtagactatggaacgtaggcgttatgatttctgacctatgtaacatggtccactaactct

SEQ ID NO: 27 - DNA, 23 bp, Artificial Sequence
  Synthetic polynucleotide
cctgaagagatggtagactatgg

SEQ ID NO: 28 - DNA, 25 bp, Artificial Sequence
  Synthetic polynucleotide
caccgcctgaagagatggtagacta

SEQ ID NO: 29 - DNA, 25 bp, Artificial Sequence
  Synthetic polynucleotide
aaactagtctaccatctcttcaggc

SEQ ID NO: 30 - DNA, 23 bp, Artificial Sequence
  Synthetic polynucleotide
gatggtagactatggaacgtagg

SEQ ID NO: 31 - DNA, 25 bp, Artificial Sequence
  Synthetic polynucleotide
caccggatggtagactatggaacgt

SEQ ID NO: 32 - DNA, 25 bp, Artificial Sequence
  Synthetic polynucleotide
aaacacgttccatagtctaccatcc

SEQ ID NO: 33 - DNA, 23 bp, Artificial Sequence
  Synthetic polynucleotide
tgtttttgacctatgtaacatgg

SEQ ID NO: 34 - DNA, 25 bp, Artificial Sequence
  Synthetic polynucleotide
caccgtgtttttgacctatgtaaca

SEQ ID NO: 35 - DNA, 25 bp, Artificial Sequence
  Synthetic polynucleotide
aaactgttacataggtcaaaaacac

SEQ ID NO: 36 - DNA, 23 bp, Artificial Sequence
  Synthetic polynucleotide
cctatgtaacatggtccactaac

SEQ ID NO: 37 - DNA, 25 bp, Artificial Sequence
  Synthetic polynucleotide
caccggttagtggaccatgttacat

SEQ ID NO: 38 - DNA, 25 bp, Artificial Sequence
  Synthetic polynucleotide
aaacatgtaacatggtccactaacc

SEQ ID NO: 39 - DNA, 23 bp, Artificial Sequence
  Synthetic polynucleotide
ccactaactctcagtatccaatc

SEQ ID NO: 40 - DNA, 25 bp, Artificial Sequence
  Synthetic polynucleotide
caccggattggatactgagagttag

SEQ ID NO: 41 - DNA, 25 bp, Artificial Sequence
  Synthetic polynucleotide
aaacctaactctcagtatccaatcc

SEQ ID NO: 42 - DNA, 40 bp, Artificial Sequence
  Synthetic polynucleotide
ttaatacgactcactatagggatggtagactatggaacgt

SEQ ID NO: 43 - DNA, 40 bp, Artificial Sequence
  Synthetic polynucleotide
ttaatacgactcactatagggttagtggaccatgttacat

SEQ ID NO: 44 - DNA, 20 bp, Artificial Sequence
  Synthetic polynucleotide
aaaagcaccgactcggtgcc

SEQ ID NO: 45 - DNA, 95 bp, Mus musculus
ggttcctgaagagatggtagactatggaacgtaggcgttatgtttttgacctatgtaacatggtccactaactctcagtatccaatccatcctcg

SEQ ID NO: 46 - DNA, 59 bp, Artificial Sequence
  Synthetic polynucleotide
ggttcctgaagagatggtagaacatggtccactaactctcagtatccaatccatcctcg

SEQ ID NO: 47 - DNA, 95 bp, Mus musculus; identical to SEQ ID NO: 45

SEQ ID NO: 48 - DNA, 59 bp, Artificial Sequence
  Synthetic polynucleotide
ggttcctgaagagtggtagaaacatggtccactaactctcagtatccaatccatcctcg

SEQ ID NO: 49 - DNA, 287 bp, Mus musculus; identical to SEQ ID NO: 25

SEQ ID NO: 50 - DNA, 291 bp, Homo sapiens; identical to SEQ ID NO: 26

SEQ ID NO: 51 - DNA, 297 bp, Artificial Sequence
  Synthetic polynucleotide
  misc_feature (1)..(1): Residue is a or absent
  misc_feature (27)..(27): Residue is a or absent
  misc_feature (28)..(28): Residue is t or absent
  misc_feature (69)..(69): Residue is c or absent
  misc_feature (80)..(80): Residue is a or absent
  misc_feature (87)..(87): Residue is a or absent
  misc_feature (107)..(107): Residue is t or absent
  misc_feature (108)..(108): Residue is a or absent
  misc_feature (109)..(109): Residue is t or absent
  misc_feature (121)..(121): Residue is g or absent
  misc_feature (122)..(122): Residue is c or absent
  misc_feature (133)..(133): Residue is c or absent
  misc_feature (155)..(155): Residue is a or absent
  misc_feature (199)..(199): Residue is t or absent
  misc_feature (202)..(202): Residue is a or absent
  misc_feature (268)..(268): Residue is c or absent
nrtytttcyragttrryrtggccttcnnctggwrrwrtwmymmctkagggkrgaggyrccscytcycynwtcaryrccrntgcmmcncattsraggagggkktgttnnngttcaccayrtnnctgcttccmantgccaaatcmrgcctcagaaangctttctggaagtgacgccarcttcagggrcaaggcccwrgttnctnggggtcarcaccrttccrtggttcctgaagagatggtagactatggaacgtaggcgttatgwtttntgacctatgtaacatggtccactaactct

SEQ ID NO: 52 - DNA, 225 bp, Mus musculus
tatagtcagcacagtggttcatttttctgagttagtgtggccttcatctggtaatgtactacctgaggggggaggtgccgcctctctttcagcaccgtgcaaccattcaaggagggtgtgttgttcaccacatctgcttcccactgccaaatcaggcctcagaaaagctttctggaagtgacgccagcttcagggacaaggcccaagtttctaggggtcaacacc

SEQ ID NO: 53 - DNA, 16 bp, Artificial Sequence
  Synthetic polynucleotide
cttcccactgccaaat

SEQ ID NO: 54 - DNA, 16 bp, Artificial Sequence
  Synthetic polynucleotide
atttggcagtgggaag

SEQ ID NO: 55 - DNA, 16 bp, Artificial Sequence
  Synthetic polynucleotide
  modified_base (1)..(16): Residues modified with phosphorothioate
  modified_base (1)..(3): Residues are LNA
  modified_base (14)..(16): Residues are LNA
tcaaaaacataacgcc

SEQ ID NO: 56 - DNA, 16 bp, Artificial Sequence
  Synthetic polynucleotide
  modified_base (1)..(16): Residues modified with phosphorothioate
  modified_base (1)..(3): Residues are LNA
  modified_base (14)..(16): Residues are LNA
cacggtgctgaaagag

SEQ ID NO: 57 - DNA, 16 bp, Artificial Sequence
  Synthetic polynucleotide
  modified_base (1)..(16): Residues modified with phosphorothioate
  modified_base (1)..(3): Residues are LNA
  modified_base (14)..(16): Residues are LNA
tgaaggccacactaac

SEQ ID NO: 58 - DNA, 16 bp, Artificial Sequence
  Synthetic polynucleotide
  modified_base (1)..(16): Residues modified with phosphorothioate
  modified_base (1)..(3): Residues are LNA
  modified_base (14)..(16): Residues are LNA
atttggcagtgggaag

SEQ ID NO: 59 - RNA, 25 nt, Artificial Sequence
  Synthetic polynucleotide
  misc_feature (24)..(24): Residue is DNA
  misc_feature (25)..(25): Residue is DNA
caucugcuucccacugccaaaucag

SEQ ID NO: 60 - RNA, 27 nt, Artificial Sequence
  Synthetic polynucleotide
cugauuuggcagugggaagcagaugug

SEQ ID NO: 61 - RNA, 25 nt, Artificial Sequence
  Synthetic polynucleotide
  misc_feature (24)..(24): Residue is DNA
  misc_feature (25)..(25): Residue is DNA
ucagcaccgugcaaccauucaagga

SEQ ID NO: 62 - RNA, 27 nt, Artificial Sequence
  Synthetic polynucleotide
uccuugaaugguugcacggugcugaaa

SEQ ID NO: 63 - RNA, 25 nt, Artificial Sequence
  Synthetic polynucleotide
  misc_feature (24)..(24): Residue is DNA
  misc_feature (25)..(25): Residue is DNA
cuucaucugguaauguacuaccuga

SEQ ID NO: 64 - RNA, 27 nt, Artificial Sequence
  Synthetic polynucleotide
ucagguaguacauuaccagaugaaggc

SEQ ID NO: 65 - RNA, 21 nt, Artificial Sequence
  Synthetic polynucleotide
ggaugcaagguaucagauggu

SEQ ID NO: 66 - RNA, 22 nt, Artificial Sequence
  Synthetic polynucleotide
uagguuauccguguugccuucg

SEQ ID NO: 67 - RNA, 22 nt, Artificial Sequence
  Synthetic polynucleotide
gaaguuguucgugguggauucg

SEQ ID NO: 68 - RNA, 21 nt, Artificial Sequence
  Synthetic polynucleotide
uucaccuacaaggagauacua

SEQ ID NO: 69 - RNA, 22 nt, Artificial Sequence
  Synthetic polynucleotide
cucuaaccgguacauuaugagu

SEQ ID NO: 70 - RNA, 23 nt, Artificial Sequence
  Synthetic polynucleotide
agguuacccgagcaacuuugcau

SEQ ID NO: 71 - RNA, 22 nt, Artificial Sequence
  Synthetic polynucleotide
uucuucacguggcgcuuacaaa

SEQ ID NO: 72 - DNA, 52 bp, Artificial Sequence
  Synthetic polynucleotide
gtcctttcctaataaaatgaggaaattgcatcgcattgtctgagtaggtgtc

SEQ ID NO: 73 - DNA, 198 bp, Artificial Sequence
  Synthetic polynucleotide
aagtgacgccagcttcagggacaaggcccaagtttctaggggtcaacaccgttccatggttcctgaagagagtcctttcctaataaaatgaggaaattgcatcgcattgtctgagtaggtgtcgacatgtaacatggtccactaactctcagtatccaatccatcctcggagggcaccccggaggtgttaccaacagc

SEQ ID NO: 74 - RNA, 22 nt, Artificial Sequence
  Synthetic polynucleotide
uguuuucaacggaaacacacua

SEQ ID NO: 75 - RNA, 22 nt, Artificial Sequence
  Synthetic polynucleotide
uauguaguaugguccacaucuu

SEQ ID NO: 76 - RNA, 22 nt, Artificial Sequence
  Synthetic polynucleotide
uucuucacgugguacaaacaaa

SEQ ID NO: 77 - RNA, 22 nt, Artificial Sequence
  Synthetic polynucleotide
agggaccccgagggagggcagg

SEQ ID NO: 78 - RNA, 22 nt, Artificial Sequence
  Synthetic polynucleotide
ugcccccuccaggaagccuucu

SEQ ID NO: 79 - DNA, 77 bp, Artificial Sequence
  Synthetic polynucleotide
ttccgtggttcctgaagagatggtagactatggaacgtaggcgttatgatttctgacctatgtaacatggtccacta

SEQ ID NO: 80 - DNA, 23 bp, Artificial Sequence
  Synthetic polynucleotide
ttccgtggttcctgaagagatgg

SEQ ID NO: 81 - DNA, 23 bp, Artificial Sequence
  Synthetic polynucleotide
ccgtggttcctgaagagatggta

SEQ ID NO: 82 - DNA, 23 bp, Artificial Sequence
  Synthetic polynucleotide
gatttctgacctatgtaacatgg

SEQ ID NO: 83 - RNA, 21 nt, Artificial Sequence
  Synthetic polynucleotide
ugcaccuaaaaggagauacua

SEQ ID NO: 84 - RNA, 22 nt, Artificial Sequence
  Synthetic polynucleotide
uaugugggaugguaaaccgcuu

SEQ ID NO: 85 - RNA, 21 nt, Artificial Sequence
  Synthetic polynucleotide
ugcaccuuaaaggagauacaa

SEQ ID NO: 86 - RNA, 21 nt, Artificial Sequence
  Synthetic polynucleotide
uguccgguagacacaauauaa

SEQ ID NO: 87 - RNA, 22 nt, Artificial Sequence
  Synthetic polynucleotide
cuccaaagggcacauacaaagu

SEQ ID NO: 88 - RNA, 22 nt, Artificial Sequence
  Synthetic polynucleotide
augguugaccauagaacaugcg

SEQ ID NO: 89 - RNA, 21 nt, Artificial Sequence
  Synthetic polynucleotide
aauaauacaugguugaucuuu

SEQ ID NO: 90 - RNA, 22 nt, Artificial Sequence
  Synthetic polynucleotide
ucucucucagacgggaacauau

SEQ ID NO: 91 - RNA, 25 nt, Artificial Sequence
  Synthetic polynucleotide
aaaggauucugcugucggucccacu

SEQ ID NO: 92 - RNA, 22 nt, Artificial Sequence
  Synthetic polynucleotide
uuucuccaauugguccacacaa

SEQ ID NO: 93 - RNA, 22 nt, Artificial Sequence
  Synthetic polynucleotide
ugucucucgaacgggaacauau

SEQ ID NO: 94 - RNA, 21 nt, Artificial Sequence
  Synthetic polynucleotide
gcaugcgauaugccagaugau

SEQ ID NO: 95 - RNA, 22 nt, Artificial Sequence
  Synthetic polynucleotide
ggggagaccaguuggucagugu

SEQ ID NO: 96 - RNA, 21 nt, Artificial Sequence
  Synthetic polynucleotide
ggaugcaagguaucagauggu

SEQ ID NO: 97 - RNA, 22 nt, Artificial Sequence
  Synthetic polynucleotide
uagguuauccguguugccuucg

SEQ ID NO: 98 - RNA, 22 nt, Artificial Sequence
  Synthetic polynucleotide
gaaguuguucgugguggauucg

SEQ ID NO: 99 - RNA, 22 nt, Artificial Sequence
  Synthetic polynucleotide
uuguaccuaaaaggagauacua

SEQ ID NO: 100 - RNA, 22 nt, Artificial Sequence
  Synthetic polynucleotide
cucuaaccgguacauuaugagu

SEQ ID NO: 101 - RNA, 23 nt, Artificial Sequence
  Synthetic polynucleotide
agguuacccgagcaacuuugcau

SEQ ID NO: 102 - RNA, 22 nt, Artificial Sequence
  Synthetic polynucleotide
uucuucacguggcgcuuacaaa

SEQ ID NO: 103 - RNA, 22 nt, Artificial Sequence
  Synthetic polynucleotide
uguuuucaacggaaacacacua

SEQ ID NO: 104 - RNA, 22 nt, Artificial Sequence
  Synthetic polynucleotide
uauguaauaugguccacaucuu

SEQ ID NO: 105 - RNA, 22 nt, Artificial Sequence
  Synthetic polynucleotide
uucuucacgugguacaaacaaa

SEQ ID NO: 106 - RNA, 22 nt, Artificial Sequence
  Synthetic polynucleotide
uaugugggaugguaaaccgcuu

SEQ ID NO: 107 - RNA, 21 nt, Artificial Sequence
  Synthetic polynucleotide
ugcacuuuaaaggagauacaa

SEQ ID NO: 108 - RNA, 21 nt, Artificial Sequence
  Synthetic polynucleotide
uguccgguagacacaauauaa

SEQ ID NO: 109 - RNA, 22 nt, Artificial Sequence
  Synthetic polynucleotide
cuccaaagggcacauacaaagu

SEQ ID NO: 110 - RNA, 22 nt, Artificial Sequence
  Synthetic polynucleotide
augguugaccauagaacaugcg

SEQ ID NO: 111 - RNA, 21 nt, Artificial Sequence
  Synthetic polynucleotide
aauaauacaugguugaucuuu

SEQ ID NO: 112 - RNA, 25 nt, Artificial Sequence
  Synthetic polynucleotide
aagggauucugauguuggucacacu

SEQ ID NO: 113 - RNA, 22 nt, Artificial Sequence
  Synthetic polynucleotide
uuuuuccaaucgacccacacaa

SEQ ID NO: 114 - RNA, 22 nt, Artificial Sequence
  Synthetic polynucleotide
ugucucucgaacgggaacauau

SEQ ID NO: 115 - RNA, 21 nt, Artificial Sequence
  Synthetic polynucleotide
gcaugcgauaugccagaugau

SEQ ID NO: 116 - RNA, 22 nt, Artificial Sequence
  Synthetic polynucleotide
uguuuucaacggaaacacacua

SEQ ID NO: 117 - DNA, 169 bp, Artificial Sequence
  Synthetic polynucleotide
ggtcaacaccgttccatggttcctgaagagaggttcctgaagagatggtagactatggaacgtaggcgttatgtttttgacctatgtaacatggtccactaactctcagtatccaatccatcctcgatgtaacatggtccactaactctcagtatccaatccatcctcg

SEQ ID NO: 118 - RNA, 287 nt, Mus musculus
auuuuucugaguuaguguggccuucaucugguaauguacuaccugaggggggaggugccgccucucuuucagcaccgugcaaccauucaaggaggguguguuguucaccacaucugcuucccacugccaaaucaggccucagaaaagcuuucuggaagugacgccagcuucagggacaaggcccaaguuucuaggggucaacaccguuccaugguuccugaagagaugguagacuauggaacguaggcguuauguuuuugaccuauguaacaugguccacuaacucu

SEQ ID NO: 119 - RNA, 291 nt, Homo sapiens
agucuuuccaaguugacauggccuuccuggaggaauuaccacuuaggguagaggcaccccuucccccaucaaugccacugccccacauuggaggagggguuguuuauguucaccaugugccugcuuccaaugccaaauccagccucagaaagcuuucuggaagugacgccaacuucaggggcaaggcccugguucuggggucagcaccauuccgugguuccugaagagaugguagacuauggaacguaggcguuaugauuucugaccuauguaacaugguccacuaacucu

SEQ ID NO: 120 - DNA, 25 bp, Artificial Sequence
  Synthetic polynucleotide
caccgttccgtggttcctgaagaga

SEQ ID NO: 121 - DNA, 25 bp, Artificial Sequence
  Synthetic polynucleotide
aaactctcttcaggaaccacggaac

SEQ ID NO: 122 - DNA, 25 bp, Artificial Sequence
  Synthetic polynucleotide
caccggatttctgacctatgtaaca

SEQ ID NO: 123 - DNA, 25 bp, Artificial Sequence
  Synthetic polynucleotide
aaactgttacataggtcagaaatcc

SEQ ID NO: 124 - DNA, 25 bp, Artificial Sequence
  Synthetic polynucleotide
caccgtaccatctcttcaggaacca

SEQ ID NO: 125 - DNA, 25 bp, Artificial Sequence
  Synthetic polynucleotide
aaactggttcctgaagagatggtac

SEQ ID NO: 126 - DNA, 25 bp, Artificial Sequence
  Synthetic polynucleotide
caccgccatagtctaccatctcttc

SEQ ID NO: 127 - DNA, 25 bp, Artificial Sequence
  Synthetic polynucleotide
aaacgaagagatggtagactatggc

SEQ ID NO: 128 - DNA, 16 bp, Artificial sequence
  Synthetic polynucleotide
ggcacatggtgaacat

SEQ ID NO: 129 - DNA, 16 bp, Artificial sequence
  Synthetic polynucleotide
acggaatggtgctgac

SEQ ID NO: 130 - DNA, 16 bp, Artificial sequence
  Synthetic polynucleotide
gtgaacataaacaacc

SEQ ID NO: 131 - DNA, 170 bp, Artificial sequence
  Synthetic polynucleotide
cacacaggaaacagctatgaccatgattacgccaagctcagaattaaccctcactaaagggactagtcctgcaggtttaaacgaattcgcccttaagggcgaattcgcggccgctaaattcaattcgccctatagtgagtcgtattacaattcactggccgtcgttttac

(19) United States
(12) Patent Application Publication
(10) Pub. No.: US 2021/0310000 A1
(43) Pub. Date: Oct. 7, 2021

(54) COMPOUNDS OF CHEMICALLY MODIFIED OLIGONUCLEOTIDES AND METHODS OF USE THEREOF

(71) Applicant: CITY OF HOPE, Duarte, CA (US)

(72) Inventors: Mitsuo KATO, Duarte, CA (US); Rama NATARAJAN, Duarte, CA (US)

(21) Appl. No.: 17/268,068

(22) PCT Filed: Aug. 16, 2019
(86) PCT No.: PCT/US19/46896

§ 371 Date: Feb. 11, 2021

Related U.S. Application Data
(60) Provisional application No. 62/719,566, filed on Aug. 17, 2018.

(51) Int. Cl. C12N 15/113; A61K 31/7125; A61P 3/10; A61P 3/04; A01K 67/027

(52) U.S. Cl. CPC C12N 15/113; A61K 31/7125; A61P 3/10; A61P 3/04; A01K 67/0278; A01K 67/0276; C12N 2310/346; A01K 2217/072; A01K 2227/105; A01K 2217/075; C12N 2310/113; C12N 2310/3231; C12N 2310/341; C12N 2310/315

(57) ABSTRACT

The present disclosure relates to isolated compounds including a nucleic acid sequence capable of hybridizing to an RNA sequence 10 to 270 nucleobases downstream of the transcription start site of a mammalian microRNA-379 transcript; methods of treating a condition of a subject (e.g., diabetes, obesity, or complications thereof) with the compounds; and methods of inhibiting expression of a mammalian microRNA-379 megacluster with the compounds.

### CROSS-REFERENCE

This application claims the benefit of priority to U.S. Provisional Patent Application Ser. No. 62/719,566, filed Aug. 17, 2018, which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant No. NIH R01 DK081705 awarded by the National Institutes of Health. The government has certain rights to this invention.

### SEQUENCE LISTING

The material in the accompanying Sequence Listing is hereby incorporated by reference in its entirety. The accompanying Sequence Listing file, named “048440-709001WO_SequenceListing_ST25.txt”, was created on Aug. 2, 2019, and is 31,674 bytes in size.

### BACKGROUND OF THE DISCLOSURE

Diabetes mellitus is a major health epidemic categorized into two subclasses: type 1, known as insulin dependent diabetes mellitus (IDDM), and type 2, noninsulin dependent diabetes mellitus (NIDDM). Type 2 diabetes is a chronic and progressive metabolic disorder of carbohydrate and lipid metabolism and accounts for nearly 90% of diabetes mellitus and results from impaired insulin secretion and reduced peripheral insulin sensitivity—a burgeoning, worldwide health problem affecting almost twenty-six million people in the United States. Deficiencies associated with currently available treatments include hypoglycemic episodes, weight gain, gastrointestinal problems, edema, and loss of responsiveness over time.

### BRIEF SUMMARY OF THE DISCLOSURE

In view of the foregoing, there is a need for alternative compositions and methods for treating diabetes. The present disclosure addresses this need, and provides additional advantages as well. In particular, various aspects and embodiments of the present disclosure provide methods and compositions for use in the treatment of diabetes (e.g., pancreatic islet dysfunction), obesity, and complications of one or more of these. In embodiments, the present disclosure provides nucleic acids that hybridize to a microRNA-379 transcript, such as a microRNA-379 transcript in a live cell, and methods of using the same.

In embodiments, the present disclosure provides a method of treating a condition of a subject, the method comprising administering to the subject an effective amount of a compound comprising a nucleic acid sequence capable of hybridizing to an RNA sequence 10 to 270 nucleobases downstream of the transcription start site of a mammalian microRNA-379 transcript, wherein (i) said nucleic acid sequence comprises a nucleobase analog or a modified internucleotide linkage, and (ii) said condition is diabetes or obesity. In embodiments, the condition is diabetes. In embodiments, the compound inhibits expression of a long non-coding RNA (lncMGC) comprising microRNA-376a, microRNA-299, microRNA-376c, microRNA-410, microRNA-494, microRNA-380-5p, microRNA-369-3p, microRNA-300, microRNA-541, microRNA-329, microRNA-381, microRNA-411, microRNA-134, microRNA-379, microRNA-154, microRNA-382, microRNA-376b, microRNA-496, microRNA-409-5p, microRNA-543, microRNA-377, microRNA-380-3p, or microRNA-495, in said subject. In embodiments, the compound inhibits expression of a microRNA-379 gene cluster. In embodiments, the nucleobase analog is at the 5′-end or the 3′-end of said nucleic acid sequence. In embodiments, the nucleic acid sequence comprises three nucleobase analogs at the 5′-end or the 3′-end of said nucleic acid sequence. In embodiments, the nucleobase analog is a Locked Nucleic Acid (LNA), 2′-O-alkyl nucleobase, 2′-Fluoro nucleobase, or 2′-OMe nucleobase. In embodiments, the RNA sequence is 11 to 27, 61 to 93, 115 to 139, or 246 to 265 nucleobases downstream of said transcription start site. In embodiments, the nucleic acid sequence comprises a modified internucleotide linkage. In embodiments, the modified internucleotide linkage is a phosphorothioate linkage. In embodiments, the nucleic acid sequence has at least 90% sequence identity with a continuous 10 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130. In embodiments, the nucleic acid sequence has at least 90% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130. In embodiments, the nucleic acid sequence is 10 to 30 nucleobases in length.

In embodiments, the present disclosure provides a use of a compound for treating diabetes or obesity in a subject, as well as use of a compound in the manufacture of a medicament for such treatment. In embodiments, the compound is a compound as disclosed herein, including compounds disclosed in connection with methods of various embodiments disclosed herein. In embodiments, the compound comprises a nucleic acid sequence capable of hybridizing to an RNA sequence 10 to 270 nucleobases downstream of the transcription start site of a mammalian microRNA-379 transcript. In embodiments, the nucleic acid sequence comprises a nucleobase analog or a modified internucleotide linkage.

In embodiments, the present disclosure provides a genetically engineered non-human animal comprising a recombinant nucleic acid molecule stably integrated into the genome of said animal. In embodiments, the recombinant nucleic acid molecule encodes an RNA sequence 10 to 270 nucleobases downstream of the transcription start site of a human microRNA-379 transcript. In embodiments, the recombinant nucleic acid differs in sequence from a corresponding wild-type nucleic acid of non-human animals of the same type. In embodiments, the non-human animal is a mouse.

Other features and advantages of the disclosure will be apparent from the following detailed description and claims.

Reference is made to US20160348105A1, which is incorporated by reference in its entirety for all purposes. Unless noted to the contrary, all publications, references, patents and/or patent applications reference herein are hereby incorporated by reference in their entirety for all purposes.

### BRIEF DESCRIPTION OF THE DRAWINGS

FIG. 1A depicts a schematic illustration of the microRNA-379 region of chromosome 12 at chr12qF1, and a diagram showing the mega cluster of microRNAs (miRNAs) and their upstream promoter region. The label “CHOP” indicates upstream binding sites for the C/EBP homologous protein (CHOP), a transcription factor (TF) associated with the ER and stress response.

FIG. 1B is a diagram of a mouse receiving an injection (e.g., subcutaneous injection) of MGC10.

FIGS. 2A-B show images of cells from streptozotocin (STZ)-injected Type 1 diabetic mice, some of which were treated with MGC10 (STZ-MGC10) and some of which were not (STZ-control).

FIGS. 3A-D are bar graphs illustrating results of example expression level analyses. Legends from top to bottom correspond to members in each group of bars from left to right. FIGS. 3A and 3B depict illustrative results for inhibition of the human homologue of lncMGC by HMGC10 in human kidney mesangial cells (HMC). FIGS. 3C-D depict illustrative results for effects of four GapmeRs targeting human lncMGC in human kidney Hk-2 cell line cells.

FIGS. 4A-B illustrate an example strategy for producing a recombinant mouse comprising a humanized lncMGC.

FIG. 5 illustrates results of a PCR analysis of F1 mice following introduction of a humanized lncMGC sequence.

FIG. 6 illustrates an example strategy for producing a recombinant mouse comprising a humanized lncMGC.

FIG. 7 is a bar graph illustrating blood glucose levels in groups of mice.

FIG. 8 shows images of tissue samples stained for islets.

FIG. 9 shows images of tissue samples stained for insulin-positive β-cells.

FIG. 10 shows images of tissue samples stained for EDEM3.

FIG. 11 shows images of tissue samples stained for CHOP.

FIGS. 12A-C are bar graphs illustrating blood glucose levels in groups of mice. FIG. 12C shows lower trends of blood glucose levels in miR-379KO mice with 50 mg/mkg×5 STZ injections. Error bars are ±SEM, “*” indicates a statistically significant difference, and “NS” indicates a statistically non-significant difference between CTR and 50 mg/kg×4 or 50 mg/kg×5 at p<0.05. In FIG. 12A in each group of two bars, bars from left to right correspond to results for wild-type and miR-379KO mice, respectively. In FIG. 12B in each group of three bars, bars from left to right correspond to results for control mice, mice with 40 mg/mkg×4 STZ injections, and mice with 40 mg/mkg×5 STZ injections, respectively.

FIG. 13 is a bar graph illustrating expression levels of lncMGC in human islets isolated from type 2 diabetics (T2D) and healthy controls.

FIG. 14 is a graph illustrating body weight among groups of mice. Error bars are ±SEM, and “*” indicates a statistically significant difference between KO-HFD and WT-HFD at p<0.05.

FIG. 15 illustrates an example strategy for crossing miR-379KO mice with Akita diabetic mice, including results of a PCR analysis of F1 mice tested for the Akita genotype.

FIG. 16A illustrates predicted target sites for miR-494 (top table) and miR-376 (bottom table) in the 3′ UTR of Mettl3.

FIG. 16B illustrates results for expression analysis of Mettl3 protein in db/db and db/+ mice.

FIGS. 17A-C illustrate comparison of Mettl3 expression levels in STZ-induced diabetic mice as compared to controls. FIG. 17A shows images of tissue samples stained for Mettl3. FIG. 17B is a graphical comparison of Mettl3 expression levels at 6 weeks after STZ injection. FIG. 17C is a graphical comparison of Mettl3 expression levels at 24 weeks after STZ injection.

FIG. 18 is a diagram of an example process for the identification of miRNA targets. Targets of miR-379 identified by this process are shown in the bottom right.

FIG. 19A-B illustrate example strategies for producing a recombinant mouse comprising a humanized lncMGC, a lncMGC knockout (KO), and targeting of humanized lncMGC with a GapmeR.

FIG. 19C illustrates example results of a PCR analysis of humanized lncMGC mice following introduction of a humanized lncMGC sequence.

FIG. 20 is a graph illustrating blood glucose levels in groups of mice.

FIG. 21 illustrates decreased Ago2-IP RNA sequence reads at Fis1 3′UTR in miR-379-KO cells.

FIG. 22 illustrates decreased Ago2-IP RNA seq reads at Txn1 3′UTR in miR-379 KO cells. Top two RNA reads are from WT cells and bottom two RNA reads are from miR-379 KO cells. The target site Txn1 is boxed, and the illustrated window spans a total of 929 base pairs in chromosome 4.

FIGS. 23A-E illustrates decreased Ago2-IP RNA seq reads at Vegfb (FIG. 23A), Slc20a1 (FIG. 23B), Hnrnpc (FIG. 23C), Clta (FIG. 23D), and Ap3s1 3′UTR (FIG. 23E) in KO cells.

FIG. 24 illustrates significant decrease of Ago2IP RNA levels in the 3′UTR of new targets in KO cells. “*” indicates P<0.05 and “**” indicates P<0.01.

FIGS. 25A-B are bar graphs illustrating significant decrease of 3′UTR (Fis1 and Txn1) luciferase reporter activity by miR-379. “*” indicates P<0.05, “**” indicates P<0.01, and “NC” indicates negative control.

FIGS. 26A-B illustrate mitochondrial activity in WT mouse mesangial cells (WT MMC) and miR-379KO MMC (KO MMC) in high glucose (HG) or low glucose (LG) conditions. In the top panel of FIG. 26A, the curves from top to bottom at 40 minutes are for WT MMC LG, KO MMC LG, KO MMC HG, and WT MMC HG, respectively. In the lower panels of FIGS. 26A-B in each group of four bars, bars from left to right correspond to results for: wild type in low glucose, wild type in high glucose, miR-379KO in low gluclose, and miR-379KO in high glucose, respectively. “****” indicates P<0.0001.

FIG. 27 shows images of glomerular mesangial cells.

FIGS. 28A-C are bar graphs illustrating body weight, total body fat, and total lean mass in diabetic WT mice and diabetic miR-379KO mice. In each group of four bars, bars from left to right correspond to results for wild-type control, wild-type treated with STZ, miR-379KO control, and miR-379KO treated with STZ, respectively.

FIGS. 29A-H show images of glomerular tissue and bar graphs illustrating inhibition of glomerular hypertrophy, fibrosis, GBM & podocyte dysfunction in diabetic miR-379KO mice. “**” indicates P<0.01, “***” indicates P<0.001, “****” indicates <0.0001. In each group of four bars in the bar graphs, bars from left to right correspond to results for wild-type control, wild-type treated with STZ, miR-379KO control, and miR-379KO treated with STZ, respectively.

FIGS. 30A-F show images of glomerular tissue and bar graphs illustrating significant decrease of EDEM3,Fis1 and Txn1 in diabetic WT mice but restored in diabetic miR-379KO mice. “*” indicates P<0.05, “**” indicates P<0.01, “****” indicates <0.0001. Data are presented as mean±SEM. In each group of four bars in the bar graphs, bars from left to right correspond to results for wild-type control, wild-type treated with STZ, miR-379KO control, and miR-379KO treated with STZ, respectively.

FIG. 31 shows transmission electron micrographs of mitochondrial structure. Black arrows indicate regular internal structure and elongated mitochondria, except for the WT STZ sample, in which black arrows indicate disrupted cristae.

FIGS. 32A-D. are bar graphs illustrating body weight of WT and miR379KO-HFD male and female mice. FIG. 32A shows male body weight gain (n=12/group) and FIG. 32B shows total body fat (n=5/group). FIG. 32C shows female body weight gain (n=6/group) and FIG. 32D total body fat (n=6/group) after 24 weeks high fat diet. Statistical analyses were performed by One-way ANOVA with post-hoc Tukey test for multiple comparisons. “*” indicates P<0.05, “**” indicates P<0.01, “****” indicates P<0.0001. Data are presented as mean±SEM.

FIG. 33A shows images of tissue from male mice stained with Periodic acid-Schiff stain (PAS).

FIG. 33B is a bar graph showing quantitative analysis of glomerular PAS positive area from male mice. n=30 in controls and n=50 glomeruli in HFD groups. Statistical analyses were performed by One-way ANOVA with post-hoc Tukey test for multiple comparisons. “*” indicates P<0.05 and “***” indicates P<0.001. All data are presented as mean±SEM.

FIG. 34A shows images of tissue from male mice stained with Masson's trichrome stain.

FIG. 34B is a bar graph showing quantitative analysis of Masson's trichrome positive area (n=10 field/group) from tissue from male mice. Statistical analyses were performed by One-way ANOVA with post-hoc Tukey test for multiple comparisons. “*” indicates P<0.05, “**” indicates P<0.01. All data are presented as mean±.

FIG. 35A shows images of tissue from female mice stained with Periodic acid-Schiff stain (PAS).

FIG. 35B is a bar graph showing quantitative analysis of glomerular PAS positive area from tissue from female mice. n=30 in controls and n=50 glomeruli in HFD groups. Statistical analyses were performed by One-way ANOVA with post-hoc Tukey test for multiple comparisons. “*” indicates P<0.05 and “***” indicates P<0.001. All data are presented as mean±SEM.

FIG. 36A show images of tissue from female mice stained with Masson's trichrome stain.

FIG. 36B is a bar graph showing quantitative analysis of Masson's trichrome positive area (n=10 field/group) from tissue from female mice. Statistical analyses were performed by One-way ANOVA with post-hoc Tukey test for multiple comparisons. “*” indicates P<0.05, “**” indicates P<0.01. All data are presented as mean±.

FIG. 37A is a plasmid map showing the position of the lncMGC sequence marked as PCR product. A portion of the double-stranded sequence is shown. The stop strand is SEQ ID NO: 131, and the bottom strand is the complement thereof.

FIG. 37B shows an SDS PAGE gel showing separation of lncMGC interacting proteins.

FIG. 37C shows identified lncMGC interacting proteins. Proteins are identified by accession number, and include the following, from top to bottom: NP_003861.1, NP_001180298.1, NP_954659.1, NP_001273490.1, NP_001302459.1, NP_003592.3, NP_055847.1, NP_066997.3, NP_542417.2, NP_001273294.1, NP_001001998.1, NP_001127911.1, NP_003971.1, NP_001099008.1, NP_055317.1, NP_001027454.1, NP_004550.2, and NP_001307896.1.

FIG. 38 is a protein-protein interaction network identifying candidate protein complexes, as illustrated by Search Tool for the Retrieval of Interacting Genes/Proteins (STRING) database, and superimposed with ovals indicated functional groups.

FIG. 39 shows in situ hybridization of MGC10 in mouse pancreas.

FIG. 40 shows images of tissue stained for insulin-positive cells.

FIG. 41A-B show images of tissue stained for insulin-positive cells (FIG. 41A) or insulin-positive cells, duct cells, and nuclei (FIG. 41B).

FIG. 42 is a bar graph illustrating lower expression of lncMGC in mouse duct progenitor cells. “**” indicates P<0.01.

FIGS. 43A-B are graphs illustrating lower blood glucose levels by GapmeR targeting lncMGC in non-obese diabetic (NOD) mice. “**” indicates P<0.001.

FIG. 44 show images of tissue stained with Haemotoxylin and Eosin (H&E) or immunofluorescence (IF).

FIG. 45A shows images of tissue stained with H&E and with varying degrees of insulitis.

FIG. 45B is a bar graph showing increase of healthy islets by GapmeR lncMGC injection. In each group of two bars, bars from left to right correspond to results for control NOD mice and NOD mice treated by GapmeR lncMGC, respectively.

FIG. 46 is a bar graph showing increase of hlncMGC by cytokines in the human β-cell line. “*” indicates P<0.05.

FIGS. 47A-B are bar graphs showing reduced human lncMGC (FIG. 47A) and miR-379 (FIG. 47B) expression in 1.1B4 cells treated with hMGC10. “***” indicates P<0.001.

FIGS. 48A-E are bar graphs showing decreased expression of miR-379 cluster miRNA members including miR411 (FIG. 48A), miR494 (FIG. 48B), miR495 (FIG. 48C), miR377 (FIG. 48D), and miR410 (FIG. 48E) in 1.1B4 cells treated with hMGC10. “***” indicates P<0.001.

### DETAILED DESCRIPTION OF THE DISCLOSURE

Provided herein is, inter alia, an isolated compound including a nucleic acid sequence capable of hybridizing to an RNA sequence 10 to 270 nucleobases downstream of the transcription start site of a mammalian microRNA-379 transcript; methods of treating a condition of a subject with the compound, wherein the condition is diabetes, obesity, or a complication thereof; and methods of inhibiting expression of a mammalian microRNA-379 megacluster.

In embodiments, the compound includes a nucleic acid sequence having a nucleobase analog. In embodiments, the nucleic acid sequence includes Locked Nucleic Acid (LNA), 2′-O-alkyl, 2′ O-Methyl, 2′-deoxy-2′fluoro, 2′-deoxy, a universal base, 5-C-methyl, an inverted deoxy abasic residue incorporation, or any combination thereof. In embodiments, the nucleic acid sequence may include analogs with positive backbones; non-ionic backbones, modified sugars, and non-ribose backbones (e.g. phosphorodiamidate morpholino oligos).

The current disclosure provides an isolated compound including a nucleic acid sequence having at least 90% sequence identity with a continuous 10 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130. The current disclosure further provides a pharmaceutical composition including a compound of this disclosure, and a pharmaceutically acceptable diluent, carrier, salt or adjuvant.

The following definitions are included for the purpose of understanding the present subject matter and for constructing the appended patent claims. Abbreviations used herein have their conventional meaning within the chemical and biological arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this disclosure. The following definitions are provided to facilitate understanding of certain terms used frequently herein and are not meant to limit the scope of the present disclosure.

“Nucleic acid” or “oligonucleotide” or “polynucleotide” or grammatical equivalents used herein means at least two nucleotides covalently linked together. The term “nucleic acid” includes single-, double-, multiple-stranded or branched DNA, RNA and analogs (derivatives) thereof.

The term “modified internucleotide linkage” or “internucleotide linkage analogue” and the like refers, in the usual and customary sense, to a non-physiologic linkage between nucleotides. For example, the term “phosphorothioate nucleic acid” refers to a nucleic acid in which one or more internucleotide linkages are through a phosphorothioate moiety (thiophosphate) moiety. The phosphorothioate moiety may be a monothiophosphate (—P(O)₃(S)³⁻—) or a dithiophosphate (—P(O)₂(S)₂³⁻—). In embodiments, one or more of the nucleosides of a phosphorothioate nucleic acid are linked through a phosphorothioate moiety (e.g. monothiophosphate) moiety, and the remaining nucleosides are linked through a phosphodiester moiety (—P(O)₄³⁻—). In embodiments, one or more of the nucleosides of a phosphorothioate nucleic acid are linked through a phosphorothioate moiety (e.g. monothiophosphate) moiety, and the remaining nucleosides are linked through a methylphosphonate linkage. In embodiments, all the nucleosides of a phosphorothioate nucleic acid are linked through a phosphorothioate moiety (e.g. a monothiophosphate) moiety.

As used herein, phosphorothioate oligonucleotides (phosphorothioate nucleic acids) are from about 5, 6, 7, 8, 9, 10, 11, 12, 13, 14, 15, 16, 17, 18, 19, 20, 21, 22, 23, 24, 25, 26, 27, 28, 29, or 30 nucleotides in length. In embodiments, the phosphorothioate nucleic acids herein contain one or more phosphodiester bonds. In other embodiments, the phosphorothioate nucleic acids include alternate backbones (e.g., mimics or analogs of phosphodiesters as known in the art, such as, boranophosphate, methylphosphonate, phosphoramidate, or O-methylphosphoroamidite linkages (see Eckstein, Oligonucleotides and Analogues: A Practical Approach, Oxford University Press).

In embodiments, the phosphorothioate nucleic acids may include one or more nucleic acid analog monomers known in the art, such as, peptide nucleic acid monomer or polymer, locked nucleic acid monomer or polymer, morpholino monomer or polymer, glycol nucleic acid monomer or polymer, or threose nucleic acid monomer or polymer. Other analog nucleic acids include those with positive backbones; non-ionic backbones, and nonribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. Phosphorothioate nucleic acids and phosphorothioate polymer backbones can be linear or branched. For example, the branched nucleic acids are repetitively branched to form higher ordered structures such as dendrimers and the like.

The terms “analog,” “nucleobase analog” and the like, in the context of nucleic acid bases refer, in the usual and customary sense, to chemical moieties that can substitute for normal (i.e., physiological) nucleobases (i.e., A, T, G, C and U) in nucleic acids. Nucleobase analogs can be categorized as purine analogs and pyrimidine analogs. Purine analogs have a core purine ring structure which is substituted to form a purine analog. Pyrimidine analogs have a core pyrimidine ring structure which is substituted to form a pyrimidine analog. Substitution may be endocyclic (i.e., within the purine or pyrimidine ring structure) or exocyclic (i.e., attached to the purine or pyrimidine ring structure). Exemplary nucleobase analogs include, but are not limited to: 1,5-dimethyluracil, 1-methyluracil, 2-amino-6-hydroxyaminopurine, 2-aminopurine, 3-methyluracil, 5-(hydroxymethyl)cytosine, 5-bromouracil, 5-carboxycytosine, 5-fluoroorotic acid, 5-fluorouracil, 5-formylcytosine, 5-formyluracil, 6-azathymine, 6-azauracil, 8-azaadenine, 8-azaguanine, N6-carbamoylmethyladenine, N6-hydroxyadenine, allopurinol, hypoxanthine, thiouracil, locked nucleic acid (LNA), 2′-O-alkyl nucleobase, 2′-Fluoro nucleobase, and 2′-OMe nucleobase.

As used herein, locked nucleic acid (LNA) is a modified RNA nucleotide. LNAs are RNA molecules which possess an extra bridge connecting the 2′ oxygen and 4′ carbon of the ribose moiety. The ribose becomes locked in the 3′-endo (North) conformation. Base stacking and backbone pre-organization are enhanced by the locked ribose conformation. In embodiments, LNA modification has several advantages, including reduced toxicity, lower dosing, higher affinity and efficient targeting.

As used herein the term “nucleobases” refers to the naturally occurring compounds, which form the differentiating component of nucleotides; five bases occur in nature, three of which are common to RNA and DNA (uracil replaces thymine in RNA). Bases are divided into two groups, purines and pyrimidines, based on their chemical structure. Purines are larger, double-ring molecules comprising adenine and guanine, whereas pyrimidines have only a single-ring structure and comprise cytosine and thymine/uracil. Because of the different size of the two types of nucleobases, purines can only base pair with pyrimidines in order to preserve the DNA molecule's constant width. More specifically, the only base pairs that will fit the structure of the particular molecule are adenine-thymine and cytosine-guanine.

The term “cell” as used herein also refers to individual cells, cell lines, or cultures derived from such cells. A “culture” refers to a composition comprising isolated cells of the same or a different type.

As used herein, “diabetes” refers herein to a group of metabolic diseases in which patients have high blood glucose levels. The term includes onset and inducement of diabetes mellitus in any manner, and includes type 1, type 2, gestational, steroid-induced, HIV treatment induced and autoimmune diabetes.

Diabetic nephropathy (DN), also known as diabetic kidney disease (DKD), is typically defined by macroalbuminuria—that is, a urinary albumin excretion of more than 300 mg in a 24-hour collection—or macroalbuminuria and abnormal renal function as represented by an abnormality in serum creatinine, calculated creatinine clearance, or glomerular filtration rate (GFR). Clinically, diabetic nephropathy is characterized by a progressive increase in proteinuria and decline in GFR, hypertension, and a high risk of cardiovascular morbidity and mortality.

As used herein, “early stage DN” or “incipient DN” is characterized by microalbuminuria, which is defined as levels of albumin ranging from 30 to 300 mg in a 24-h urine collection. Microalbuminuria progresses to overt nephropathy. Renal disease is suspected to be secondary to diabetes in the clinical setting of long-standing diabetes. This is supported by the history of diabetic retinopathy, particularly in type 1 diabetics, in whom there is a strong correlation. The natural history of diabetic nephropathy is a process that progresses gradually over years.

Renal biopsy findings consistent with diabetic nephropathy in the early stages of DN are mesangial expansion and glomerular basement membrane thickening. Eventual progression of diabetic nephropathy can lead to nodular glomerulosclerosis, also referred to as Kimmelstiel-Wilson disease.

Early diabetic nephropathy is heralded by glomerular hyperfiltration and an increase in GFR. This is believed to be related to increased cell growth and expansion in the kidneys, possibly mediated by hyperglycemia itself. Microalbuminuria typically occurs after 5 years in type 1 diabetes. Overt nephropathy, with urinary protein excretion higher than 300 mg/day, often develops after 10 to 15 years. ESRD develops in 50% of type 1 diabetics, with overt nephropathy within 10 years.

Kidney disease in type 2 diabetes has a more variable course. Patients often present at diagnosis with microalbuminuria because of delays in diagnosis and other factors affecting protein excretion. Fewer patients with microalbuminuria progress to advanced renal disease. Without intervention, approximately 30% progress to overt nephropathy and, after 20 years of nephropathy, approximately 20% develop ESRD. Because of the high prevalence of type 2 compared with type 1 diabetes, however, most diabetics on dialysis are type 2 diabetics.

Long-standing hyperglycemia is known to be a significant risk factor for the development of diabetic nephropathy. Hyperglycemia may directly result in mesangial expansion and injury by an increase in the mesangial cell glucose concentration. The glomerular mesangium expands initially by cell proliferation and then by cell hypertrophy. Increased mesangial stretch and pressure can stimulate this expansion, as can high glucose levels. Transforming growth factor 13 (TGF-β) is particularly important in the mediation of expansion and later fibrosis via the stimulation of collagen and fibronectin. Glucose can also bind reversibly and eventually irreversibly to proteins in the kidneys and circulation to form advanced glycosylation end products (AGEs). AGEs can form complex cross-links over years of hyperglycemia and can contribute to renal damage by stimulation of growth and fibrotic factors via receptors for AGEs. In addition, mediators of proliferation and expansion, including platelet-derived growth factor, TGF-β, and vascular endothelial growth factor (VEGF) that are elevated in diabetic nephropathy can contribute to further complications.

Proteinuria, a marker and potential contributor to renal injury, accompanies diabetic nephropathy. Increased glomerular permeability will allow plasma proteins to escape into the urine. Some of these proteins will be taken up by the proximal tubular cells, which can initiate an inflammatory response that contributes to interstitial scarring eventually leading to fibrosis. Tubulointerstitial fibrosis is seen in advanced stages of diabetic nephropathy and is a better predictor of renal failure than glomerular sclerosis. Hyperglycemia, angiotensin II, TGF-0, and likely proteinuria itself all play roles in stimulating this fibrosis. There is an epithelial-mesenchymal transition that takes place in the tubules, with proximal tubular cell conversion to fibroblast-like cells. These cells can then migrate into the interstitium and produce collagen and fibronectin.

In diabetic nephropathy, the activation of the local renin-angiotensin system occurs in the proximal tubular epithelial cells, mesangial cells, and podocytes. Angiotensin II (ATII) itself contributes to the progression of diabetic nephropathy. ATII is stimulated in diabetes despite the high-volume state typically seen with the disease, and the intrarenal level of ATII is typically high, even in the face of lower systemic concentrations. ATII preferentially constricts the efferent arteriole in the glomerulus, leading to higher glomerular capillary pressures. In addition to its hemodynamic effects, ATII also stimulates renal growth and fibrosis through ATII type 1 receptors, which secondarily upregulate TGF-β and other growth factors.

Control of hypertension has clearly shown to be an important and powerful intervention in decreasing the progression of diabetic nephropathy. In diabetics who have disordered autoregulation at the level of the kidney, systemic hypertension can contribute to endothelial injury. Human studies of type 2 diabetics have shown that blood pressure lowering, regardless of the agent used, retards the onset and progression of diabetic nephropathy. In animal studies, the degree and severity of the diabetic nephropathy were strongly linked to systemic blood pressure.

The fact that most types 1 and 2 diabetics do not develop diabetic nephropathy (DN) suggests that other factors may be involved. Genetic factors clearly play a role in the predisposition to diabetic nephropathy in family members who have DN, and linkage to specific areas on the human genome is evolving. The theory of a reduction in nephron number at birth indicates that individuals born with a reduced number of glomeruli may be predisposed to subsequent renal injury and progressive nephropathy. This has been shown in animal studies in which the mother was exposed to hyperglycemia at the time of pregnancy. If this linkage is true in humans, that would have important implications concerning the role of maternal factors in the eventual development of kidney disease.

Diabetic nephropathy (DN) includes the expansion and hypertrophy of glomerular mesangial cells (MCs), increased accumulation of extracellular matrix (ECM) proteins such as collagen 1alpha1 (Col1α1), Col1α2, Col4α1 and fibronectin, and tubulointerstitial fibrosis, podocyte dysfunction and proteinuria. Levels of transforming growth factor-beta1 (TGF-β1) are increased in MCs and other renal cells in diabetics and TGF-β1 mediates many of the adverse effects. Several biochemical mechanisms of action have been reported for TGF-β1. Factors relevant to the pathogenesis of DN such as angiotensin II, and high glucose (HG), increase TGF-β1 expression in MCs in vitro and in vivo. Signals from the activated TGF-β1 receptor complex are transduced to the nucleus by Smad proteins, including Smad2/3/4, which regulate TGF-β-induced genes, including PAI-1, collagen and p21cip1/waf1. However, the molecular mechanisms by which diabetic conditions and TGF-β1 regulate the genes that increase the hypertrophy, protein synthesis and fibrosis associated with DN are not fully clear. A few microRNAs (miRNAs or miRs, in short) are involved in mediating the pro-fibrotic effects of TGF-β1 in MCs in vitro and diabetic conditions in vivo.

microRNAs (miRNA) are endogenously produced, short single-stranded non-coding RNAs (˜20-23 nucleotides) that play key roles in post-transcriptional regulation of gene expression to silence genes by repressing the translation or inducing the degradation of target mRNAs. There are more than 1000 mammalian miRNAs that can target nearly 60% of mRNAs in the genome, and therefore, they regulate many key cellular functions. The terms microRNA, miRNA, and miR are interchangeable.

Long non-coding RNAs (lncRNAs) are long transcripts that range from >200 nucleotides up to −100 kb, and are similar to messenger RNAs (mRNAs) but lack protein coding (translation) potential. LncRNAs can regulate the expression of local and distal genes by various mechanisms that include recruiting histone modifying complexes and modulating the activities of transcription factors (TFs). LncRNAs also serve as hosts for miRNAs and/or a miRNA megacluster. LncRNAs have cell-specific expression, and function in various biological processes including transcription, differentiation, and the immune response.

As used herein, the microRNA megacluster is a region of the genome where more than 10 microRNA genes are encoded. In embodiments, 35-60 microRNAs are encoded in the region. In embodiments, some of these clustered miRNA genes may be encoded by a single-copy DNA sequence. Alternatively, the miRNA genes may be arranged in tandem arrays of closely related sequences.

As used herein, the microRNA-379 (miR-379) transcript is a RNA sequence transcribed from a microRNA-379 gene of a mammalian genome, e.g., a human genome. In its ordinary meaning, a “transcript” in molecular biology or similar context is a product of transcription. miRNAs are transcribed as much larger primary transcripts (pri-miRNAs). The vast majority of mature miRNAs are produced from primary transcripts of microRNAs (pri-miRNAs) by a multi-step pathway. In mammals, miRNAs are first transcribed as longer primary transcripts called primary miRNA (pri-miRNA). The transcript may contain multiple miRNA stem loops and is capped at the 5′ end through polyadenylation. Drosha, a nuclear RNase III, is recruited to crop the pri-miRNA transcript into a hairpin-shaped structure, about 70 nt long, known as precursor-miRNA (pre-miRNA). This cleavage event is critical and site-specific, as it determines the mature miRNA sequence. The pre-miRNA is then exported out of the nucleus for further cleavage into a 22 nt duplex. The complementary strand becomes degraded leaving one fully mature miRNA strand. Mature miRNA then associate with several members of the Argonaute protein family to form the RNA-induced silencing complex which then binds to specific protein-coding mRNA transcripts, directing mRNA inactivation by translational repression, deadenylation, or degradation. In embodiments, the miR-379 transcript is a mouse transcript. The sequence of the mouse miR-379 transcript including the upstream region of mouse miR-379 and the mouse miR-379 sequence is shown in SEQ ID NO: 118. In embodiments, the miR-379 transcript is a human transcript. The sequence of the human miR-379 transcript including the upstream region of human miR-379 and the human miR-379 sequence is shown in SEQ ID NO: 119.

As used herein, plasminogen activator inhibitor-1 (PAI-1) is an endothelial plasminogen activator inhibitor or serpin μl is a protein that in humans is encoded by the SERPINE1 gene. PAI-1 is a serine protease inhibitor (serpin) that functions as the principal inhibitor of tissue plasminogen activator (tPA) and urokinase (uPA), the activators of plasminogen and hence fibrinolysis (the physiological breakdown of blood clots). It is a serine protease inhibitor (serpin) protein (SERPINE1). Other PAI, plasminogen activator inhibitor-2 (PAI-2) is secreted by the placenta and only present in significant amounts during pregnancy. In addition, protease nexin acts as an inhibitor of tPA and urokinase. PAI-1, however, is the main inhibitor of the plasminogen activators.

As used herein, connective-tissue growth factor (CTGF) is a secreted protein implicated in multiple cellular events including angiogenesis, skeletogenesis and wound healing.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

“Patient,” “subject,” “patient in need thereof,” and “subject in need thereof” are herein used interchangeably and refer to a living organism suffering from or prone to a disease or condition that can be treated by administration using the methods and compositions provided herein. Non-limiting examples include humans, other mammals, bovines, rats, mice, dogs, monkeys, goat, sheep, cows, deer, and other non-mammalian animals. In some embodiments, a patient is human. Tissues, cells and their progeny of a biological entity obtained in vitro or cultured in vitro are also contemplated.

The terms “treat,” “treating” or “treatment,” and other grammatical equivalents as used herein, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

The terms “prevent,” “preventing,” or “prevention,” and other grammatical equivalents as used herein, include to keep from developing, occur, hinder or avert a disease or condition symptoms as well as to decrease the occurrence of symptoms. The prevention may be complete (i.e., no detectable symptoms) or partial, so that fewer symptoms are observed than would likely occur absent treatment. The terms further include a prophylactic benefit. For a disease or condition to be prevented, the compositions may be administered to a patient at risk of developing a particular disease, or to a patient reporting one or more of the physiological symptoms of a disease, even though a diagnosis of this disease may not have been made.

The term “inhibiting” also means reducing an effect (disease state or expression level of a gene/protein/mRNA) relative to the state in the absence of a compound or composition of the present disclosure.

A “test compound” as used herein refers to an experimental compound used in a screening process to identify activity, non-activity, or other modulation of a particularized biological target or pathway.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

“Disease” or “condition” refer to a state of being or health status of a patient or subject capable of being treated with the compounds or methods provided herein. In some instances, “disease” or “condition” refers to diabetes, obesity, or a complication thereof.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture. In some embodiments contacting includes allowing a compound described herein to interact with a protein or enzyme.

The terms “phenotype” and “phenotypic” as used herein refer to an organism's observable characteristics such as onset or progression of disease symptoms, biochemical properties, or physiological properties.

The word “expression” or “expressed” as used herein in reference to a DNA nucleic acid sequence (e.g. a gene) means the transcriptional and/or translational product of that sequence. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). When used in reference to polypeptides, expression includes any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “promoter” and the like in the usual and customary sense, is a region of DNA that initiates transcription of a particular gene. Promoters are located near the transcription start sites of genes, on the same strand and upstream on the DNA (towards the 5′ region of the sense strand). Upstream and downstream in the usual and customary sense both refer to a relative position in DNA or RNA. Each strand of DNA or RNA has a 5′ end and a 3′ end, so named for the carbon position on the deoxyribose (or ribose) ring. By convention, upstream and downstream relate to the 5′ to 3′ direction in which RNA transcription takes place. Upstream is toward the 5′ end of the RNA molecule and downstream is toward the 3′ end. When considering double-stranded DNA, upstream is toward the 5′ end of the coding strand for the gene in question and downstream is toward the 3′ end. Due to the anti-parallel nature of DNA, this means the 3′ end of the template strand is upstream of the gene and the 5′ end is downstream.

The term “an amount of” in reference to a polynucleotide or polypeptide, refers to an amount at which a component or element is detected. The amount may be measured against a control, for example, wherein an increased level of a particular polynucleotide or polypeptide in relation to the control, demonstrates enrichment of the polynucleotide or polypeptide. The term refers to quantitative measurement of the enrichment as well as qualitative measurement of an increase or decrease relative to a control.

Throughout the description and claims of this specification the word “comprise” and other forms of the word, such as “comprising” and “comprises,” means including but not limited to, and is not intended to exclude, for example, other components.

“Analog,” “analogue,” or “derivative” is used in accordance with its plain ordinary meaning within Chemistry and Biology and refers to a chemical agent that is structurally similar to another agent (i.e., a so-called “reference” agent) but differs in composition, e.g., in the replacement of one atom by an atom of a different element, or in the presence of a particular functional group, or the replacement of one functional group by another functional group, or the absolute stereochemistry of a chiral center of the reference agent. In some embodiments, a derivative may be a conjugate with a pharmaceutically acceptable agent, for example, phosphate or phosphonate.

As used herein, the term “salt” refers to acid or base salts of the agents used herein. Illustrative but non-limiting examples of acceptable salts are mineral acid (hydrochloric acid, hydrobromic acid, phosphoric acid, sulfuric acid, and the like) salts, organic acid (acetic acid, propionic acid, glutamic acid, citric acid, and the like) salts, and quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, which is combined with buffer prior to use.

Thus, the compounds of the present disclosure may exist as salts, such as with pharmaceutically acceptable acids. The present disclosure includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

An “adjuvant” (from Latin, adiuvare: to aid) is a pharmacological and/or immunological agent that modifies the effect of other agents.

A “diluent” (also referred to as a filler, dilutant or thinner) is a diluting agent. Certain fluids are too viscous to be pumped easily or too dense to flow from one particular point to the other. This can be problematic, because it might not be economically feasible to transport such fluids in this state. To ease this restricted movement, diluents are added. This decreases the viscosity of the fluids, thereby also decreasing the pumping/transportation costs.

The terms “administration” or “administering” refer to the act of providing an agent of the current embodiments or pharmaceutical composition including an agent of the current embodiments to the individual in need of treatment.

By “co-administer” it is meant that a composition described herein is administered at the same time, just prior to, or just after the administration of additional therapies. The compound or the composition of the disclosure can be administered alone or can be co-administered to the patient. Co-administration is meant to include simultaneous or sequential administration of the compound individually or in combination (more than one compound or agent). The preparations can also be combined, when desired, with other active substances (e.g. to reduce metabolic degradation).

As used herein, “sequential administration” includes that the administration of two agents (e.g., the compounds or compositions described herein) occurs separately on the same day or do not occur on a same day (e.g., occurs on consecutive days).

As used herein, “concurrent administration” includes overlapping in duration at least in part. For example, when two agents (e.g., any of the agents or class of agents described herein that has bioactivity) are administered concurrently, their administration occurs within a certain desired time. The agents' administration may begin and end on the same day. The administration of one agent can also precede the administration of a second agent by day(s) as long as both agents are taken on the same day at least once. Similarly, the administration of one agent can extend beyond the administration of a second agent as long as both agents are taken on the same day at least once. The bioactive agents/agents do not have to be taken at the same time each day to include concurrent administration.

As used herein, “intermittent administration” includes the administration of an agent for a period of time (which can be considered a “first period of administration”), followed by a time during which the agent is not taken or is taken at a lower maintenance dose (which can be considered an “off-period”) followed by a period during which the agent is administered again (which can be considered a “second period of administration”). Generally, during the second phase of administration, the dosage level of the agent will match that administered during the first period of administration but can be increased or decreased as medically necessary.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and. Other modes of delivery include, but are not limited to, the use of liposomal formulations, intravenous infusion, transdermal patches, etc.

The compositions disclosed herein can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions disclosed herein can also be delivered as microspheres for slow release in the body. For example, microspheres can be administered via intradermal injection of drug-containing microspheres, which slowly release subcutaneously (see Rao, J. Bioniater Sci. Polym. Ed. 7:623-645, 1995; as biodegradable and injectable gel formulations (see, e.g., Gao Phann. Res. 12:857-863, 1995); or, as microspheres for oral administration (see, e.g., Eyles, J. Phann. Pharmacol. 49:669-674, 1997).

As used herein, an “effective amount” or “therapeutically effective amount” is that amount sufficient to affect a desired biological effect, such as beneficial results, including clinical results. As such, an “effective amount” depends upon the context in which it is being applied. An effective amount may vary according to factors known in the art, such as the disease state, age, sex, and weight of the individual being treated. Several divided doses may be administered daily or the dose may be proportionally reduced as indicated by the exigencies of the therapeutic situation. In addition, the compositions/formulations of this disclosure can be administered as frequently as necessary to achieve a therapeutic amount.

Pharmaceutical compositions may include compositions wherein the therapeutic drug (e.g., agents described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of therapeutic drug effective to achieve the desired result, e.g., modulating the activity of a target molecule, and/or reducing, eliminating, or slowing the progression of disease symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and agents of this disclosure. Adjustment and manipulation of established dosages (e.g., frequency and duration) are well within the ability of those skilled in the art.

For any therapeutic agent described herein, the therapeutically effective amount can be initially determined from cell culture assays. Target concentrations will be those concentrations of therapeutic drug(s) that are capable of achieving the methods described herein, as measured using the methods described herein or known in the art.

Therapeutically effective amounts for use in humans can also be determined from animal models. For example, a dose for humans can be formulated to achieve a concentration that has been found to be effective in animals. The dosage in humans can be adjusted by monitoring agent's effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the therapeutic drug being employed. The dose administered to a patient should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the agent. Thereafter, the dosage is increased by small increments until the optimum effect under circumstances is reached. Dosage amounts and intervals can be adjusted individually to provide levels of the administered agent effective for the particular clinical indication being treated. This will provide a therapeutic regimen that is commensurate with the severity of the individual's disease state.

A weight percent of a component, unless specifically stated to the contrary, is based on the total weight of the formulation or composition in which the component is included.

“Excipient” is used herein to include any other agent that may be contained in or combined with a disclosed agent, in which the excipient is not a therapeutically or biologically active agent/agent. As such, an excipient should be pharmaceutically or biologically acceptable or relevant (for example, an excipient should generally be non-toxic to the individual). “Excipient” includes a single such agent and is also intended to include a plurality of excipients. For the purposes of the present disclosure the term “excipient” and “carrier” are used interchangeably in some embodiments of the present disclosure and said terms are defined herein as, “ingredients which are used in the practice of formulating a safe and effective pharmaceutical composition.”

The term “about” refers to any minimal alteration in the concentration or amount of an agent that does not change the efficacy of the agent in preparation of a formulation and in treatment of a disease or disorder. The term “about” with respect to concentration range of the agents (e.g., therapeutic/active agents) of the current disclosure also refers to any variation of a stated amount or range which would be an effective amount or range.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Compounds

The present disclosure includes an isolated compound including a nucleic acid sequence capable of hybridizing to an RNA sequence 10 to 270 nucleobases downstream of the transcription start site of a mammalian microRNA-379 transcript or a microRNA-379 megacluster transcript. In embodiments, the present disclosure includes an isolated compound including a nucleic acid sequence capable of hybridizing to at least one nucleic acid base of a downstream region of the transcription start site of a mammalian microRNA-379 transcript or a microRNA-379 megacluster transcript. In embodiments, the transcript is as exists immediately after transcription, e.g., primary transcript mRNA or pre-mRNA. In embodiments, the compound includes a nucleic acid sequence having a nucleobase analog or modified internucleotide linkage.

In embodiments, the compound includes a nucleic acid sequence having a nucleobase analog. In embodiments, the nucleic acid sequence includes Locked Nucleic Acid (LNA), 2′-O-alkyl, 2′ O-Methyl, 2′-deoxy-2′fluoro, 2′-deoxy, a universal base, 5-C-methyl, an inverted deoxy abasic residue incorporation, or any combination thereof. In embodiments, the nucleic acid sequence may include analogs with positive backbones; non-ionic backbones, modified sugars, and non-ribose backbones (e.g. phosphorodiamidate morpholino oligos), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids.

In embodiments, the nucleic acid sequence includes at least one nucleic acid analog. In embodiments, the nucleic acid sequence includes at least one nucleic acid analog having an alternate backbone (e.g. phosphodiester derivative (e.g. phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, or O-methylphosphoroamidite), peptide nucleic acid backbone(s), LNA, or linkages). In embodiments, a nucleic acid sequence includes or is DNA. In embodiments, a nucleic acid sequence includes or is RNA. In embodiments, a nucleic acid sequence includes or is a nucleic acid having internucleotide linkages selected from phosphodiesters and phosphodiester derivatives (e.g., phosphoramidate, phosphorodiamidate, phosphorothioate, phosphorodithioate, phosphonocarboxylic acids, phosphonocarboxylates, phosphonoacetic acid, phosphonoformic acid, methyl phosphonate, boron phosphonate, O-methylphosphoroamidite, or combinations thereof). In embodiments, a nucleic acid sequence consists of a nucleic acid having internucleotide linkages selected from phosphodiesters and phosphorothioates. In embodiments, a nucleic acid sequence includes or is a nucleic acid having backbone linkages selected from phosphodiesters and phosphorodithioates. In embodiments, a nucleic acid sequence includes or is a nucleic acid having phosphodiester backbone linkages. In embodiments, a nucleic acid sequence includes or is a nucleic acid having phosphorothioate backbone linkages. In embodiments, a nucleic acid sequence includes or is a nucleic acid having phosphorodithioate backbone linkages.

In embodiments, a nucleic acid sequence in the compound includes a nucleic acid analog (e.g. LNA, 2′-O-alkyl, 2′-Fluoro, or 2′ O-Methyl (2′-OMe)) at 1, 2, 3, 4, 5, 6, 7, 8, 9, or 10 nucleobases. In embodiments, the compound includes a nucleic acid sequence capable of hybridizing to an RNA sequence 10 to 270 nucleobases downstream of the transcription start site of a mammalian microRNA-379 transcript, where the nucleic acid sequence has an analog (e.g., LNA, 2′-O-alkyl, 2′-Fluoro, or 2′ O-Methyl (2′-OMe)) at 1, 2, 3, 4, 5, 6, 7, 8, 9, or 10 nucleobases. In embodiments, the compound includes a nucleic acid sequence with an analog (e.g., LNA, 2′-O-alkyl, 2′-Fluoro, or 2′ O-Methyl (2′-OMe)) at 3 nucleobases.

In embodiments, the nucleobase analog is at the 5′-end or the 3′-end of the nucleic acid sequence. In embodiments, the nucleobase analog (e.g., LNA, 2′-O-alkyl, 2′-Fluoro, or 2′ O-Methyl (2′-OMe)) is at the 5′-end or the 3′-end of the nucleic acid sequence. In embodiments, the nucleobase analog (e.g., LNA, 2′-O-alkyl, 2′-Fluoro, or 2′-OMe) is at the 5′-end and the 3′-end of the nucleic acid sequence.

In embodiments, the nucleic acid sequence includes three, four or five nucleobase analogs (e.g., LNA, 2′-O-alkyl, 2′-Fluoro, or 2′ O-Methyl (2′-OMe)) at the 5′-end or the 3′-end of the nucleic acid sequence. In embodiments, the nucleic acid sequence includes three, four or five nucleobase analogs (e.g., LNA, 2′-O-alkyl, 2′-Fluoro, or 2′ O-Methyl (2′-OMe)) at the 5′-end and the 3′-end of the nucleic acid sequence. In embodiments, the nucleic acid sequence includes three nucleobase analogs (e.g., LNA, 2′-O-alkyl, 2′-Fluoro, or 2′ O-Methyl (2′-OMe)) at the 5′-end or the 3′-end of the nucleic acid sequence. In embodiments, the nucleic acid sequence includes three nucleobase analogs (e.g., LNA, 2′-O-alkyl, 2′-Fluoro, or 2′ O-Methyl (2′-OMe)) at the 5′-end and the 3′-end of the nucleic acid sequence

In embodiments, the compound includes a nucleic acid sequence with a modified internucleotide linkage. In embodiments, the modified internucleotide linkage is a phosphorothioate (also known as phosphothioate) linkage. In other embodiments, nucleic acid analogs are included that may have alternate backbones (e.g. phosphodiester derivatives), including, e.g., phosphoramidate, phosphorodiamidate, phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

In embodiments, the compound includes a nucleic acid sequence with internal modified internucleotide linkage between nucleobases at one or more positions. In embodiments, the compound includes a nucleic acid sequence with internal modified internucleotide linkage between nucleobases at one or more positions, and one, two, three, or four nucleobase analogs at the 5′- or the 3′-ends of the nucleic acid sequence. In embodiments, the compound includes a nucleic acid sequence with internal internucleotide phosphorothioate linkage between nucleobases at one or more positions, and one, two, three, or four nucleobase LNA analogs at the 5′- or the 3′-ends of the nucleic acid sequence. In embodiments, the compound includes a nucleic acid sequence with internal modified internucleotide linkage between nucleobases at one or more positions, and one, two, three, or four nucleobase analogs at the 5′- and the 3′-ends of the nucleic acid sequence. In embodiments, the compound includes a nucleic acid sequence with internal internucleotide phosphorothioate linkage between nucleobases at one or more positions, and one, two, three, or four nucleobase LNA analogs at the 5′- and the 3′-ends of the nucleic acid sequence.

Structures of exemplary molecules for internucleotide analogues, such as an LNA monomer, and internucleotide linkages, such as phosphodiester linkage and phosphorothioate linkage, are depicted below.

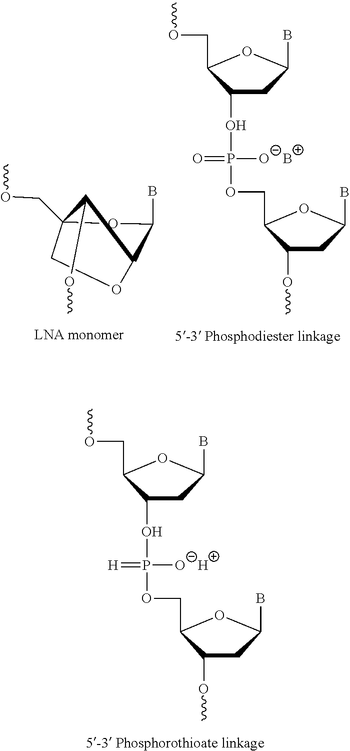

In embodiments, the RNA sequence to which a nucleic acid sequence of the present disclosure hybridizes includes 11 to 27, 61 to 93, 115 to 139, or 246 to 265 nucleobases downstream of the transcription start site of the gene. In embodiments, the target site on the RNA sequence to which a nucleic acid sequence of the present disclosure hybridizes to is listed in Table 1. The target site range listed in Table 1 (middle column) reflects the nucleobase positions counting from the transcription start site at +1 of a target RNA.

TABLE 1

| Nucleic | | |
|---|---|---|
| Acid | | |
| Identity | Target site | Nucleic acid sequence |
| MGC8 | +11 to +26 | TGAAGGCCACACTAAC |
| | | (SEQ ID NO: 1) |
| MGC12 | +12 to +27 | ATGAAGGCCACACTAA |
| | | (SEQ ID NO: 2) |
| MGC15 | +11 to +25 | GAAGGCCACACTAAC |
| | | (SEQ ID NO: 3) |
| MGC5 | +64 to +79 | CACGGTGCTGAAAGAG |
| | | (SEQ ID NO: 4) |
| MGC6 | +63 to +78 | ACGGTGCTGAAAGAGA |
| | | (SEQ ID NO: 5) |
| MGC13 | +63 to +77 | CGGTGCTGAAAGAGA |
| | | (SEQ ID NO: 6) |
| MGC14 | +78 to +93 | TCCTTGAATGGTTGCA |
| | | (SEQ ID NO: 7) |
| MGC18 | +75 to +90 | TTGAATGGTTGCACGG |
| | | (SEQ ID NO: 8) |
| MGC20 | +62 to +77 | CGGTGCTGAAAGAGAG |
| | | (SEQ ID NO: 9) |
| MGC10 | +117 to +132 | ATTTGGCAGTGGGAAG |
| | | (SEQ ID NO: 10) |
| MGC17 | +116 to +131 | TTTGGCAGTGGGAAGC |
| | | (SEQ ID NO: 11) |
| MGC19 | +115 to +130 | TTGGCAGTGGGAAGCA |
| | | (SEQ ID NO: 12) |
| MGC1 | +246 to +261 | TCAAAAACATAACGCC |
| | | (SEQ ID NO: 13) |
| MGC2 | +247 to +262 | GTCAAAAACATAACGC |
| | | (SEQ ID NO: 14) |
| MGC3 | +248 to +262 | GGTCAAAAACATAACGC |
| | | (SEQ ID NO: 15) |
| MGC4 | +248 to +263 | GGTCAAAAACATAACG |
| | | (SEQ ID NO: 16) |
| MGC7 | +249 to +264 | AGGTCAAAAACATAAC |
| | | (SEQ ID NO: 17) |
| MGC9 | +249 to +263 | AGGTCAAAAACATAACG |
| | | (SEQ ID NO: 18) |
| MGC11 | +251 to +265 | TAGGTCAAAAACATA |
| | | (SEQ ID NO: 19) |
| MGC16 | +246 to +260 | CAAAAACATAACGCC |
| | | (SEQ ID NO: 20) |
| HMGC10 | +124 to +139 | GATTTGGCATTGGAAG |
| | | (SEQ ID NO: 21) |
| HMGC8 | +12 to +27 | GGAAGGCCATGTCAAC |
| | | (SEQ ID NO: 22) |
| HMGC5 | +61 to +76 | GGCATTGATGGGGGAA |
| | | (SEQ ID NO: 23) |
| HMGC1 | +249 to +265 | TCAGAAATCATAACGCC |
| | | (SEQ ID NO: 24) |
| HMGCN1 | +106 to +121 | GGCACATGGTGAACAT |
| | | (SEQ ID NO: 128) |
| HMGCN2 | +200 to +215 | ACGGAATGGTGCTGAC |
| | | (SEQ ID NO: 129) |
| HMGCN4 | +98 to +113 | GTGAACATAAACAACC |
| | | (SEQ ID NO: 130) |

In embodiments, the compound includes, e.g., GATTTGGCATTGGAAG (SEQ ID NO: 21) with internal internucleotide phosphorothioate linkage between one or more nucleobases, and one, two, three, or four nucleobase LNA analogs at the 5′- and/or the 3′-ends of the nucleic acid sequence. In embodiments, the LNA analogs at the 5′ and/or the 3′-ends of the sequence are underlined, e.g., GATTTGGCATTGGAAG (SEQ ID NO: 21). In embodiments, the remaining internal internucleotide linkages between nucleobases (italicized in the above sequence) are phosphorothioate linkages. In embodiments, a compound is, e.g., GATTTGGCATTGGAAG (SEQ ID NO: 21), with internal internucleotide phosphorothioate linkage between one or more nucleobases.

In embodiments, the compound includes a nucleic acid that binds to the mouse miR-379 transcript including the upstream region of mouse miR-379 and the mouse miR-379 sequence. The sequence of the mouse miR-379 transcript including the upstream region of mouse miR-379 and the mouse miR-379 sequence is shown in SEQ ID NO: 118. The nucleic acid sequences of SEQ ID NOs: 1-20 hybridize to a region of mouse miR-379 transcript of SEQ ID NO: 25 (the transcription start site indicated with “+1”), i.e., SEQ ID NO: 118; in SEQ ID NO: 118, a uracil (“U”) replaces each thymine (“T”) of SEQ ID NO: 25.

+1
(SEQ ID NO: 25)
ATTTTTCTGAGTTAGTGTGGCCTTCATCTGGTAATGTACTACCTGAGGGGG
GAGGTGCCGCCTCTCTTTCAGCACCGTGCAACCATTCAAGGAGGGTGTGTT
GTTCACCACATCTGCTTCCCACTGCCAAATCAGGCCTCAGAAAAGCTTTCT
GGAAGTGACGCCAGCTTCAGGGACAAGGCCCAAGTTTCTAGGGGTCAACAC
CGTTCCATGGTTCCTGAAGAGATGGTAGACTATGGAACGTAGGCGTTATGT
TTTTGACCTATGTAACATGGTCCACTAACTCT
+1
(SEQ ID NO: 118)
AUUUUUCUGAGUUAGUGUGGCCUUCAUCUGGUAAUGUACUACCUGAGGGGG
GAGGUGCCGCCUCUCUUUCAGCACCGUGCAACCAUUCAAGGAGGGUGUGUU
GUUCACCACAUCUGCUUCCCACUGCCAAAUCAGGCCUCAGAAAAGCUUUCU
GGAAGUGACGCCAGCUUCAGGGACAAGGCCCAAGUUUCUAGGGGUCAACAC
CGUUCCAUGGUUCCUGAAGAGAUGGUAGACUAUGGAACGUAGGCGUUAUGU
UUUUGACCUAUGUAACAUGGUCCACUAACUCU

In embodiments, the compound includes a nucleic acid that binds to the human miR-379 transcript including the upstream region of human miR-379 and the human miR-379 sequence. The sequence of the human miR-379 transcript including the upstream region of human miR-379 and the human miR-379 sequence is shown in SEQ ID NO: 119. The nucleic acid sequences of SEQ ID NOs: 21-24 and 128-130 hybridize to a region of human miR-379 transcript of SEQ ID NO: 26 (the transcription start site indicated with “+1”), i.e., SEQ ID NO: 119; in SEQ ID NO: 119, a uracil (“U”) replaces each thymine (“T”) of SEQ ID NO: 26.

In embodiments, a nucleic acid sequence having 90-91%, 91-92%, 92-93%, 93-94%, 94-95%, 95-96%, 96-97%, 97-98%, or 98-99% sequence identity with a continuous 10 nucleobase sequence of SEQ ID NO: 1, 2, 3, 4, 5, 6, 7, 8, 9, 10, 11, 12, 13, 14, 15, 16, 17, 18, 19, or 20 hybridizes to a region of human miR-379 transcript of SEQ ID NO: 26, i.e., SEQ ID NO: 119; in SEQ ID NO: 119, a uracil (“U”) replaces each thymine (“T”) of SEQ ID NO: 26.

+1
(SEQ ID NO: 26)
AGTCTTTCCAAGTTGACATGGCCTTCCTGGAGGAATTACCACTTAGGGTAG
AGGCACCCCTTCCCCCATCAATGCCACTGCCCCACATTGGAGGAGGGGTTG
TTTATGTTCACCATGTGCCTGCTTCCAATGCCAAATCCAGCCTCAGAAAGC
TTTCTGGAAGTGACGCCAACTTCAGGGGCAAGGCCCTGGTTCTGGGGTCAG
CACCATTCCGTGGTTCCTGAAGAGATGGTAGACTATGGAACGTAGGCGTTA
TGATTTCTGACCTATGTAACATGGTCCACTAACTCT.
+1
(SEQ ID NO: 119)
AGUCUUUCCAAGUUGACAUGGCCUUCCUGGAGGAAUUACCACUUAGGGUAG
AGGCACCCCUUCCCCCAUCAAUGCCACUGCCCCACAUUGGAGGAGGGGUUG
UUUAUGUUCACCAUGUGCCUGCUUCCAAUGCCAAAUCCAGCCUCAGAAAGC
UUUCUGGAAGUGACGCCAACUUCAGGGGCAAGGCCCUGGUUCUGGGGUCAG
CACCAUUCCGUGGUUCCUGAAGAGAUGGUAGACUAUGGAACGUAGGCGUUA
UGAUUUCUGACCUAUGUAACAUGGUCCACUAACUCU

The consensus sequence of the mouse and human miR-379 transcript corresponds to a transcript of the consensus sequence provided in SEQ ID NO: 51.

In embodiments, the compound includes a nucleic acid sequence that is 10 to 30 nucleobases in length. In embodiments, the compound includes a nucleic acid sequence capable of hybridizing at least 5, 6, 7, 8, 9, 10, 11, 12, 13, 14, 15, 16, 17, 18, 19, 20, 21, 22, 23, 24, 25, 26, 27, 28, 29, or 30 nucleobases within a RNA sequence 10 to 270 nucleobases downstream of the transcription start site of a mammalian microRNA-379 transcript. In embodiments, the mammalian microRNA-379 transcript is a human microRNA-379 transcript. In embodiments, the compound includes a nucleic acid sequence capable of hybridizing at least 5, 6, 7, 8, 9, 10, 11, 12, 13, 14, 15, 16, 17, 18, 19, 20, 21, 22, 23, 24, 25, 26, 27, 28, 29, or 30 nucleobases within the sequence of SEQ ID NO: 118 or 119. In embodiments, the compound includes a nucleic acid sequence capable of hybridizing at least 5, 6, 7, 8, 9, 10, 11, 12, 13, 14, 15, 16, 17, 18, 19, 20, 21, 22, 23, 24, 25, 26, 27, 28, 29, or 30 nucleobases within 10-20, 10-30, 20-40, 20-50, 40-60, 40-70, 60-80, 60-90, 80-100, 80-110, 100-120, 100-130, 120-140, 120-150, 140-160, 140-170, 160-180, 160-190, 180-200, 180-210, 200-220, 200-230, 220-240, 220-230, 240-260, or 240-270 nucleobases downstream of the transcription start site (indicated with “+1”) of the transcript sequence of SEQ ID NO: 25 or 26 (i.e., transcript sequence SEQ ID NO: 118 or 119), a sequence including the transcript of SEQ ID NO: 25 or 26 (i.e., transcript sequence SEQ ID NO: 118 or 119), or a variation thereof. In embodiments, the compound includes a nucleic acid sequence capable of hybridizing at least 5 nucleobases within the sequence of 10-20, 10-30, 20-40, 20-50, 40-60, 40-70, 60-80, 60-90, 80-100, 80-110, 100-120, 100-130, 120-140, 120-150, 140-160, 140-170, 160-180, 160-190, 180-200, 180-210, 200-220, 200-230, 220-240, 220-230, 240-260, or 240-270 nucleobases downstream of the transcription start site (indicated with “+1”) of the transcript of SEQ ID NO: 25 or 26 (i.e., transcript sequence SEQ ID NO: 118 or 119).

In embodiments, the compound includes a nucleic acid sequence capable of hybridizing within the sequence of nucleobases 10-20 of SEQ ID NO. 119. In embodiments, the compound includes a nucleic acid sequence capable of hybridizing within the sequence of nucleobases 10-30 of SEQ ID NO. 119. In embodiments, the compound includes a nucleic acid sequence capable of hybridizing within the sequence of nucleobases 20-40 of SEQ ID NO. 119. In embodiments, the compound includes a nucleic acid sequence capable of hybridizing within the sequence of nucleobases 20-50 of SEQ ID NO. 119. In embodiments, the compound includes a nucleic acid sequence capable of hybridizing within the sequence of nucleobases 40-60 of SEQ ID NO. 119. In embodiments, the compound includes a nucleic acid sequence capable of hybridizing within the sequence of nucleobases 40-70 of SEQ ID NO. 119. In embodiments, the compound includes a nucleic acid sequence capable of hybridizing within the sequence of nucleobases 60-80 of SEQ ID NO. 119. In embodiments, the compound includes a nucleic acid sequence capable of hybridizing within the sequence of nucleobases 60-90 of SEQ ID NO. 119. In embodiments, the compound includes a nucleic acid sequence capable of hybridizing within the sequence of nucleobases 80-100 of SEQ ID NO. 119. In embodiments, the compound includes a nucleic acid sequence capable of hybridizing within the sequence of nucleobases 80-110 of SEQ ID NO. 119. In embodiments, the compound includes a nucleic acid sequence capable of hybridizing within the sequence of nucleobases 100-120 of SEQ ID NO. 119. In embodiments, the compound includes a nucleic acid sequence capable of hybridizing within the sequence of nucleobases 100-130 of SEQ ID NO. 119. In embodiments, the compound includes a nucleic acid sequence capable of hybridizing within the sequence of nucleobases 120-140 of SEQ ID NO. 119. In embodiments, the compound includes a nucleic acid sequence capable of hybridizing within the sequence of nucleobases 120-150 of SEQ ID NO. 119. In embodiments, the compound includes a nucleic acid sequence capable of hybridizing within the sequence of nucleobases 140-160 of SEQ ID NO. 119. In embodiments, the compound includes a nucleic acid sequence capable of hybridizing within the sequence of nucleobases 140-170 of SEQ ID NO. 119. In embodiments, the compound includes a nucleic acid sequence capable of hybridizing within the sequence of nucleobases 160-180 of SEQ ID NO. 119. In embodiments, the compound includes a nucleic acid sequence capable of hybridizing within the sequence of nucleobases 160-190 of SEQ ID NO. 119. In embodiments, the compound includes a nucleic acid sequence capable of hybridizing within the sequence of nucleobases 180-200 of SEQ ID NO. 119. In embodiments, the compound includes a nucleic acid sequence capable of hybridizing within the sequence of nucleobases 180-210 of SEQ ID NO. 119. In embodiments, the compound includes a nucleic acid sequence capable of hybridizing within the sequence of nucleobases 200-220 of SEQ ID NO. 119. In embodiments, the compound includes a nucleic acid sequence capable of hybridizing within the sequence of nucleobases 200-230 of SEQ ID NO. 119. In embodiments, the compound includes a nucleic acid sequence capable of hybridizing within the sequence of nucleobases 220-240 of SEQ ID NO. 119. In embodiments, the compound includes a nucleic acid sequence capable of hybridizing within the sequence of nucleobases 220-230 of SEQ ID NO. 119. In embodiments, the compound includes a nucleic acid sequence capable of hybridizing within the sequence of nucleobases 240-260 of SEQ ID NO. 119. In embodiments, the compound includes a nucleic acid sequence capable of hybridizing within the sequence of nucleobases 240-270 of SEQ ID NO. 119.

In embodiments, the compound includes a nucleic acid sequence capable of hybridizing at least 5 nucleobases within a RNA sequence 10 to 270 nucleobases downstream of the transcription start site of a mammalian microRNA-379 transcript. In embodiments, the compound includes a nucleic acid sequence capable of hybridizing at least 5 nucleobases within a RNA sequence 10-20, 10-30, 20-40, 20-50, 40-60, 40-70, 60-80, 60-90, 80-100, 80-110, 100-120, 100-130, 120-140, 120-150, 140-160, 140-170, 160-180, 160-190, 180-200, 180-210, 200-220, 200-230, 220-240, 220-230, 240-260, or 240-270 nucleobases downstream of the transcription start site of a mammalian microRNA-379 transcript.

In embodiments, the compound includes a nucleic acid sequence capable of hybridizing at least 10 nucleobases within the miR-379 transcript. In embodiments, the compound includes a nucleic acid sequence capable of hybridizing at least 15 nucleobases within the miR-379 transcript. In embodiments, the compound hybridizes within the sequence of nucleobases 124-139 of SEQ ID NO. 119. In embodiments, the compound hybridizes within the sequence of nucleobases 12-27 of SEQ ID NO. 119. In embodiments, the compound hybridizes within the sequence of nucleobases 249-265 of SEQ ID NO. 119. In embodiments, the compound hybridizes within the sequence of nucleobases 106-121 of SEQ ID NO. 119. In embodiments, the compound hybridizes within the sequence of nucleobases 200-215 of SEQ ID NO. 119. In embodiments, the compound hybridizes within the sequence of nucleobases 98-113 of SEQ ID NO. 119.

In embodiments, the compound includes a nucleic acid sequence having at least 90% identity with the sequence of SEQ ID NO: 21. In embodiments, the compound includes a nucleic acid sequence having at least 90% identity with the sequence of SEQ ID NO: 22. In embodiments, the compound includes a nucleic acid sequence having at least 90% identity with the sequence of SEQ ID NO: 23. In embodiments, the compound includes a nucleic acid sequence having at least 90% identity with the sequence of SEQ ID NO: 24. In embodiments, the compound includes a nucleic acid sequence having at least 90% identity with the sequence of SEQ ID NO: 128. In embodiments, the compound includes a nucleic acid sequence having at least 90% identity with the sequence of SEQ ID NO: 129. In embodiments, the compound includes a nucleic acid sequence having at least 90% identity with the sequence of SEQ ID NO: 130. In embodiments, the compound includes a nucleic acid sequence of SEQ ID NO: 21. In embodiments, the compound includes a nucleic acid sequence of SEQ ID NO: 22. In embodiments, the compound includes a nucleic acid sequence of SEQ ID NO: 23. In embodiments, the compound includes a nucleic acid sequence of SEQ ID NO: 24. In embodiments, the compound includes a nucleic acid sequence of SEQ ID NO: 128. In embodiments, the compound includes a nucleic acid sequence of SEQ ID NO: 129. In embodiments, the compound includes a nucleic acid sequence of SEQ ID NO: 130.

In embodiments, the present disclosure includes a compound including a nucleic acid sequence having 90-91%, 91-92%, 92-93%, 93-94%, 94-95%, 95-96%, 96-97%, 97-98%, 98-99%, or 99-100% sequence identity with a continuous 10 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, or analogues or derivatives thereof. In embodiments, the compound includes a nucleic acid sequence having at least 90% identity with a continuous 10 nucleobase sequence of SEQ ID NO: 21. In embodiments, the compound includes a nucleic acid sequence having at least 90% identity with a continuous 10 nucleobase sequence of SEQ ID NO: 22. In embodiments, the compound includes a nucleic acid sequence having at least 90% identity with a continuous 10 nucleobase sequence of SEQ ID NO: 23. In embodiments, the compound includes a nucleic acid sequence having at least 90% identity with a continuous 10 nucleobase sequence of SEQ ID NO: 24. In embodiments, the compound includes a nucleic acid sequence having at least 90% identity with a continuous 10 nucleobase sequence of SEQ ID NO: 128. In embodiments, the compound includes a nucleic acid sequence having at least 90% identity with a continuous 10 nucleobase sequence of SEQ ID NO: 129. In embodiments, the compound includes a nucleic acid sequence having at least 90% identity with a continuous 10 nucleobase sequence of SEQ ID NO: 130.

In embodiments, the compound includes a nucleic acid sequence having 90-91%, 91-92%, 92-93%, 93-94%, 94-95%, 95-96%, 96-97%, 97-98%, 98-99%, or 99-100% sequence identity with a continuous 10 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal modified internucleotide linkage between nucleobases and/or terminal nucleobase analogs at the 5′- and/or the 3′-ends of the nucleic acid sequence.

In embodiments, the compound includes a nucleic acid sequence having 90-91%, 91-92%, 92-93%, 93-94%, 94-95%, 95-96%, 96-97%, 97-98%, 98-99%, or 99-100% sequence identity with a continuous 10 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal internucleotide phosphorothioate linkage between nucleobases and/or terminal nucleobase LNA analogs at the 5′- and/or the 3′-ends of the nucleic acid sequence. In embodiments, the nucleobase analogs at the 5′- and/or the 3′ ends may be 2′-O-alkyl nucleobase, 2′-Fluoro nucleobase, or 2′-OMe nucleobase.

In embodiments, the present disclosure includes a compound including a nucleic acid sequence having at least 90% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130. In embodiments, the compound includes a nucleic acid sequence having at least 90% identity with a continuous 15 nucleobase sequence of SEQ ID NO: 21. In embodiments, the compound includes a nucleic acid sequence having at least 90% identity with a continuous 15 nucleobase sequence of SEQ ID NO: 22. In embodiments, the compound includes a nucleic acid sequence having at least 90% identity with a continuous 15 nucleobase sequence of SEQ ID NO: 23. In embodiments, the compound includes a nucleic acid sequence having at least 90% identity with a continuous 15 nucleobase sequence of SEQ ID NO: 24. In embodiments, the compound includes a nucleic acid sequence having at least 90% identity with a continuous 15 nucleobase sequence of SEQ ID NO: 128. In embodiments, the compound includes a nucleic acid sequence having at least 90% identity with a continuous 15 nucleobase sequence of SEQ ID NO: 129. In embodiments, the compound includes a nucleic acid sequence having at least 90% identity with a continuous 15 nucleobase sequence of SEQ ID NO: 130. In embodiments, the compound includes a nucleic acid sequence having at least 90% identity with a continuous 16 nucleobase sequence of SEQ ID NO: 21. In embodiments, the compound includes a nucleic acid sequence having at least 90% identity with a continuous 16 nucleobase sequence of SEQ ID NO: 22. In embodiments, the compound includes a nucleic acid sequence having at least 90% identity with a continuous 16 nucleobase sequence of SEQ ID NO: 23. In embodiments, the compound includes a nucleic acid sequence having at least 90% identity with a continuous 16 nucleobase sequence of SEQ ID NO: 24. In embodiments, the compound includes a nucleic acid sequence having at least 90% identity with a continuous 16 nucleobase sequence of SEQ ID NO: 128. In embodiments, the compound includes a nucleic acid sequence having at least 90% identity with a continuous 16 nucleobase sequence of SEQ ID NO: 129. In embodiments, the compound includes a nucleic acid sequence having at least 90% identity with a continuous 16 nucleobase sequence of SEQ ID NO: 130.

In embodiments, the compound includes a nucleic acid sequence having at least 90% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal modified internucleotide linkage between nucleobases and/or terminal nucleobase analogs at the 5′- and/or the 3′-ends of the nucleic acid sequence.

In embodiments, the compound includes a nucleic acid sequence having at least 90% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal internucleotide phosphorothioate linkage between nucleobases and/or nucleobase LNA analogs at the 5′- and/or the 3′-ends of the nucleic acid sequence. In embodiments, the nucleobase analogs at the 5′- and the 3′ ends may be 2′-O-alkyl nucleobase, 2′-Fluoro nucleobase, or 2′-OMe nucleobase, and any combination thereof.

In embodiments, the present disclosure includes a compound including a nucleic acid sequence having 90-91%, 91-92%, 92-93%, 93-94%, 94-95%, 95-96%, 96-97%, 97-98%, 98-99%, or 99-100% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130.

In embodiments, the compound includes a nucleic acid sequence having 90-91%, 91-92%, 92-93%, 93-94%, 94-95%, 95-96%, 96-97%, 97-98%, 98-99%, or 99-100% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal modified internucleotide linkage between nucleobases and/or terminal nucleobase analogs at the 5′- and/or the 3′-ends of the nucleic acid sequence.

In embodiments, the compound includes a nucleic acid sequence having 90-91%, 91-92%, 92-93%, 93-94%, 94-95%, 95-96%, 96-97%, 97-98%, 98-99%, or 99-100% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal internucleotide phosphorothioate linkage between nucleobases and/or nucleobase LNA analogs at the 5′- and/or the 3′-ends of the nucleic acid sequence. In embodiments, the nucleobase analogs at the 5′- and/or the 3′ ends may be 2′-O-alkyl nucleobase, 2′-Fluoro nucleobase, or 2′-OMe nucleobase, and any combination thereof

Complexes

In embodiments, the present disclosure provides a complex of a compound including a nucleic acid sequence described in this disclosure hybridized to an RNA sequence 10 to 270 nucleobases downstream of the transcription start site of a mammalian microRNA-379 transcript or a microRNA-379 megacluster transcript.

In embodiments, the present disclosure includes a nucleic acid sequence of SEQ ID NOs: 1-20 hybridized to a region of mouse miR-379 transcript of SEQ ID NO: 25 (i.e., transcript sequence SEQ ID NO: 118) to form a complex. In embodiments, the present disclosure includes a nucleic acid sequence of SEQ ID NOs: 21-24 and 128-130 hybridized to a region of human miR-379 transcript of SEQ ID NO: 26 (i.e., transcript sequence SEQ ID NO: 119) to form a complex. In embodiments, the present disclosure includes a nucleic acid sequence having 90-91%, 91-92%, 92-93%, 93-94%, 94-95%, 95-96%, 96-97%, 97-98%, or 98-99% sequence identity with a continuous 10 nucleobase sequence of SEQ ID NO: 1, 2, 3, 4, 5, 6, 7, 8, 9, 10, 11, 12, 13, 14, 15, 16, 17, 18, 19, or 20, or analogues thereof, hybridized to a region of human miR-379 transcript of SEQ ID NO: 26 (i.e., transcript sequence SEQ ID NO: 119) to form a complex.

In embodiments, the present disclosure includes a complex of a nucleic acid sequence having 90-91%, 91-92%, 92-93%, 93-94%, 94-95%, 95-96%, 96-97%, 97-98%, 98-99%, or 99-100% sequence identity with a continuous 10, 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal internucleotide phosphorothioate linkage between nucleobases and/or nucleobase LNA analogs at the 5′- and/or the 3′-ends of the nucleic acid sequence, hybridized to a RNA sequence 10 to 270 nucleobase downstream of the transcription start site of microRNA-379 transcript.

In embodiments, the present disclosure includes a complex of a nucleic acid sequence having 90-91%, 91-92%, 92-93%, 93-94%, 94-95%, 95-96%, 96-97%, 97-98%, 98-99%, or 99-100% sequence identity with a continuous 10, 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal internucleotide phosphorothioate linkage between nucleobases and/or nucleobase LNA analogs at the 5′- and/or the 3′-ends of the nucleic acid sequence, hybridized to a RNA sequence transcript at 10-20, 10-30, 20-40, 20-50, 40-60, 40-70, 60-80, 60-90, 80-100, 80-110, 100-120, 100-130, 120-140, 120-150, 140-160, 140-170, 160-180, 160-190, 180-200, 180-210, 200-220, 200-230, 220-240, 220-230, 240-260, or 240-270 nucleobases downstream of the transcription start site of microRNA-379.

Methods of Treatment or Use

The present disclosure provides a method of treating a condition of a subject in need thereof, the method comprising administering to the subject an effective amount of a compound of the present disclosure, wherein the condition is diabetes, obesity, or a complication thereof. In embodiments, the condition is diabetes (e.g., type 1 diabetes or type 2 diabetes). The present disclosure includes a method of treating the condition in a subject by administering to the subject about 0.001 mg/kg to about 100 mg/kg of a compound of the present disclosure. In embodiments, a compound of the present disclosure lowers blood glucose, protects against shrinking or loss of islets, decreases β-cell death, reduces insulitis, regenerates islet cells, reduces body weight, or has a combination of two or more of these effects, relative to a control or relative to a starting level, thereby treating the condition. In embodiments, treatment comprises protecting or regenerating islet cells.

In embodiments, the method of treating the condition in a subject includes administering to the subject a compound or a pharmaceutical composition including a nucleic acid sequence having 90-91%, 91-92%, 92-93%, 93-94%, 94-95%, 95-96%, 96-97%, 97-98%, 98-99%, or 99-100% sequence identity with a continuous 10, 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, or analogues thereof.

In embodiments, the method of treating the condition in a subject includes administering to the subject a compound or a pharmaceutical composition including a nucleic acid sequence having a nucleobase analog. In embodiments, the nucleic acid sequence includes Locked Nucleic Acid (LNA), 2′-O-alkyl, 2′ O-Methyl, 2′-deoxy-2′fluoro, 2′-deoxy, a universal base, 5-C-methyl, an inverted deoxy abasic residue incorporation, or any combination thereof. In embodiments, the nucleic acid sequence may include analogs with positive backbones; non-ionic backbones, modified sugars, and non-ribose backbones (e.g. phosphorodiamidate morpholino oligos).

In embodiments, the present disclosure includes a method of treating the condition by administering a compound to a subject in need of such treatment, where the compound inhibits expression of a long non-coding RNA (lncMGC) in the subject. The method of treating the condition is by administering a compound to a subject in need of such treatment, where the compound inhibits expression of a long non-coding RNA (lncMGC) in the subject, which includes microRNA-376a, microRNA-299, microRNA-376c, microRNA-410, microRNA-494, microRNA-380-5p, microRNA-369-3p, microRNA-300, microRNA-541, microRNA-329, microRNA-381, microRNA-411, microRNA-134, microRNA-379, microRNA-154, microRNA-382, microRNA-376b, microRNA-496, microRNA-409-5p, microRNA-543, microRNA-377, microRNA-380-3p, and/or microRNA-495.

In embodiments, the present disclosure includes a method of treating the condition by administering a compound to a subject, where the compound inhibits expression of a microRNA gene cluster. In embodiments, expression of the microRNA gene cluster that is inhibited for treating the condition is microRNA-379 gene cluster. In embodiments, the microRNA gene cluster expression of which is inhibited expresses microRNAs such as microRNA-376a, microRNA-299, microRNA-376c, microRNA-410, microRNA-494, microRNA-380-5p, microRNA-369-3p, microRNA-300, microRNA-541, microRNA-329, microRNA-381, microRNA-411, microRNA-134, microRNA-379, microRNA-154, microRNA-382, microRNA-376b, microRNA-496, microRNA-409-5p, microRNA-543, microRNA-377, microRNA-380-3p, and/or microRNA-495.

The sequence of the nucleic acid that inhibits the microRNA for treating diabetic nephropathy is complementary to the microRNA sequence, or complementary to a transcript that includes the targeted microRNA and binds downstream of the transcription start site.

Human microRNAs targeted for treating diabetic nephropathy are listed in Table 2.

TABLE 2
Human microRNAs

| Name | Sequence | SEQ ID NO: |
|---|---|---|
| microRNA-376a | UGCACCUAAAAGGAGAUACUA | 83 |
| microRNA-299-3p | UAUGUGGGAUGGUAAACCGCUU | 84 |
| microRNA-376c | UGCACCUUAAAGGAGAUACAA | 85 |
| microRNA-410 | UGUCCGGUAGACACAAUAUAA | 86 |
| microRNA-494 | CUCCAAAGGGCACAUACAAAGU | 87 |
| microRNA-380-5p | AUGGUUGACCAUAGAACAUGCG | 88 |
| microRNA-369-3p | AAUAAUACAUGGUUGAUCUUU | 89 |
| microRNA-300 | UCUCUCUCAGACGGGAACAUAU | 90 |
| microRNA-541 | AAAGGAUUCUGCUGUCGGUCCCACU | 91 |
| microRNA-329 | UUUCUCCAAUUGGUCCACACAA | 92 |
| microRNA-381 | UGUCUCUCGAACGGGAACAUAU | 93 |
| microRNA-411 | GCAUGCGAUAUGCCAGAUGAU | 94 |
| microRNA-134 | GGGGAGACCAGUUGGUCAGUGU | 95 |
| microRNA-379 | GGAUGCAAGGUAUCAGAUGGU | 96 |
| microRNA-154 | UAGGUUAUCCGUGUUGCCUUCG | 97 |
| microRNA-382 | GAAGUUGUUCGUGGUGGAUUCG | 98 |
| microRNA-376b | UUGUACCUAAAAGGAGAUACUA | 99 |
| microRNA-496 | CUCUAACCGGUACAUUAUGAGU | 100 |
| microRNA-409-5p | AGGUUACCCGAGCAACUUUGCAU | 101 |
| microRNA-543 | UUCUUCACGUGGCGCUUACAAA | 102 |
| microRNA-377 | UGUUUUCAACGGAAACACACUA | 103 |
| microRNA-380-3p | UAUGUAAUAUGGUCCACAUCUU | 104 |
| microRNA-495 | UUCUUCACGUGGUACAAACAAA | 105 |

In embodiments, mouse microRNA targeted for inhibition are listed in Table 3.

TABLE 3
Mouse microRNAs:

| Name | Sequence | SEQ ID NO: |
|---|---|---|
| microRNA-299-3p | UAUGUGGGAUGGUAAACCGCUU | 106 |
| microRNA-376c | UGCACUUUAAAGGAGAUACAA | 107 |
| microRNA-410 | UGUCCGGUAGACACAAUAUAA | 108 |
| microRNA-494 | CUCCAAAGGGCACAUACAAAGU | 109 |
| microRNA-380-5p | AUGGUUGACCAUAGAACAUGCG | 110 |
| microRNA-369-3p | AAUAAUACAUGGUUGAUCUUU | 111 |
| microRNA-541 | AAGGGAUUCUGAUGUUGGUCACACU | 112 |
| microRNA-329 | UUUUUCCAAUCGACCCACACAA | 113 |
| microRNA-381 | UGUCUCUCGAACGGGAACAUAU | 114 |
| microRNA-411 | GCAUGCGAUAUGCCAGAUGAU | 115 |
| microRNA-134 | UGUUUUCAACGGAAACACACUA | 116 |
| microRNA-379 | GGAUGCAAGGUAUCAGAUGGU | 65 |
| microRNA-154 | UAGGUUAUCCGUGUUGCCUUCG | 66 |
| microRNA-382 | GAAGUUGUUCGUGGUGGAUUCG | 67 |
| microRNA-376b | UUCACCUACAAGGAGAUACUA | 68 |
| microRNA-496 | CUCUAACCGGUACAUUAUGAGU | 69 |
| microRNA-409-5p | AGGUUACCCGAGCAACUUUGCAU | 70 |
| microRNA-543 | UUCUUCACGUGGCGCUUACAAA | 71 |
| microRNA-377 | UGUUUUCAACGGAAACACACUA | 74 |
| microRNA-380-3p | UAUGUAGUAUGGUCCACAUCUU | 75 |
| microRNA-495 | UUCUUCACGUGGUACAAACAAA | 76 |
| miR-3072-5p | AGGGACCCCGAGGGAGGGCAGG | 77 |
| miR-3072-3p | UGCCCCCUCCAGGAAGCCUUCU | 78 |

In embodiments, the present disclosure includes a method of treating the condition by administering a compound of the present disclosure, which upregulates microRNA target genes and down-regulates expression of profibrotic genes. In embodiments, the compound of the present disclosure up-regulates and down-regulates in kidney mesangial cells. In embodiments, miRNA target genes are unregulated in pancreatic cells, e.g. islet cells.

In embodiments, the compound of the present disclosure up-regulates target genes, for example, Tnrc6, CUGBP2, CPEB4, Pumillio2, BHC80, EDEM3, Fis1, Clathrin, Vegf-β, thioredoxin, Hnrnpc, Mettl3, CLTA, AP3S1, TXN1, SLC20A1, or any combination(s) thereof. In embodiments, at least 2, 3, 4, 5, 10, 15, or more of these target genes are upregulated. In embodiments, all of these target genes are upregulated. In embodiments, upregulation is at least 5%, 10%, 15%, 20%, 25%, 50%, or more relative to pre-administration levels. In embodiments, the compound of the present disclosure down-regulates profibrotic genes, for example, pro-fibrotic genes Col1α2, TGF-β1, Col1α4, Plasminogen activator inhibitor-1 (PAI-1), fibronectin, connective tissue growth factor (CTGF), and any combination(s) thereof. In embodiments, 2, 3, 4, 5, or more of the pro-fibrotic genes are down-regulated. In embodiments, all of these pro-fibrotic genes are down-regulated. In embodiments, down-regulation is at least 5%, 10%, 15%, 20%, 25%, 50%, or more relative to pre-administration levels. In embodiments, the compound of the present disclosure treats the condition at an early stage of disease.

In embodiments, the condition is diabetes. In embodiments, treating the condition includes lowering blood glucose in a subject with diabetes, or slowing a rise in blood glucose in a subject having or at risk for developing diabetes. In embodiments, treating the condition includes preventing or slowing loss of pancreatic islets or of β-cells.

In embodiments, the condition is obesity. In embodiments, treating the condition includes reducing or slowing the increase in fat mass or body weight of a subject.

Methods of Inhibiting Expression of a Mammalian MicroRNA-379 Cluster

The present disclosure provides a method of inhibiting expression of a mammalian microRNA-379, the method includes hybridizing a compound of the present disclosure to an RNA sequence 10 to 270 nucleobases downstream of the transcription start site of a mammalian microRNA-379 transcript. In embodiments, the method of inhibiting expression of a mammalian microRNA-379 cluster includes contacting a cell or tissue with a nucleic acid sequence of SEQ ID NOs: 1-24 or 128-130. In embodiments, the method of inhibiting expression of a mammalian microRNA-379 cluster includes contacting a cell or tissue with a nucleic acid sequence having 90-91%, 91-92%, 92-93%, 93-94%, 94-95%, 95-96%, 96-97%, 97-98%, 98-99%, or 99-100% sequence identity with a continuous 10, 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal internucleotide phosphorothioate linkage between nucleobases and/or nucleobase LNA analogs at the 5′- and/or the 3′-ends of the nucleic acid sequence.

In embodiments, the method of inhibiting expression of a mammalian microRNA-379 cluster includes contacting a kidney mesangial cell or a pancreatic β cell with a nucleic acid sequence of SEQ ID NOs: 1-24 or 128-130. In embodiments, the method of inhibiting expression of a mammalian microRNA-379 cluster includes contacting a kidney mesangial cell or a pancreatic β cell with a nucleic acid sequence having 90-91%, 91-92%, 92-93%, 93-94%, 94-95%, 95-96%, 96-97%, 97-98%, 98-99%, or 99-100% sequence identity with a continuous 10, 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal internucleotide phosphorothioate linkage between nucleobases and/or nucleobase LNA analogs at the 5′- and/or the 3′-ends of the nucleic acid sequence.

Pharmaceutical Compositions

The present disclosure provides a pharmaceutical composition including a compound of the present disclosure and a pharmaceutically acceptable diluent, carrier, salt, and/or adjuvant.

In embodiments, the pharmaceutical composition of the present disclosure includes a nucleic acid sequence of SEQ ID NOs: 1-24 or 128-130. In embodiments, the pharmaceutical composition of the present disclosure includes a nucleic acid sequence having 90-91%, 91-92%, 92-93%, 93-94%, 94-95%, 95-96%, 96-97%, 97-98%, 98-99%, or 99-100% sequence identity with a continuous 10, 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130, with internal internucleotide phosphorothioate linkage between nucleobases and/or nucleobase LNA analogs at the 5′- and/or the 3′-ends of the nucleic acid sequence.

In embodiments, the present disclosure includes administering to an individual, a composition of a therapeutically effective amount of a compound including a nucleic acid sequence of SEQ ID NOs: 1-24 or 128-130, alone or in combination with a diabetic and/or diabetic nephropathic agent. The effective dose of the composition may be between about 0.001 mg/kg to about 100 mg/kg of compound. In embodiments, the compositions may have between about 0.1% to about 20% of the pharmaceutical composition. In embodiments, the compositions may include pharmaceutically acceptable diluent(s), excipient(s), and/or carrier(s).

The composition of a compound including a nucleic acid sequence of SEQ ID NOs: 1-24 or 128-130 may be administered with a suitable pharmaceutical carrier. The administration can be local or systemic, including oral, parenteral, intraperitoneal, intrathecal or topical application. The release profiles of such composition may be rapid release, immediate release, controlled release or sustained release. For example, the composition may comprise a sustained release matrix and a therapeutically effective amount. Alternatively, a composition of a compound including a nucleic acid sequence of SEQ ID NOs: 1-24 or 128-130 can be secreted by genetically modified cells that are implanted, either free or in a capsule, at the gut of a subject. In embodiments, a composition of a compound including a nucleic acid sequence of SEQ ID NOs: 1-24 or 128-130 may be administered to a subject via subcutaneous route. In embodiments, the composition may be administered as an oral nutritional supplement.

Oral compositions may include an inert diluent or an edible pharmaceutically acceptable carrier. They can be enclosed in gelatin capsules or compressed into tablets. For the purpose of oral administration, a composition of a compound including a nucleic acid sequence of SEQ ID NOs: 1-24 or 128-130 can be incorporated with excipients and used in the form of tablets, troches, or capsules. Oral compositions can also be prepared using a fluid carrier for use as a mouthwash, wherein the agent in the fluid carrier is applied orally and swished and expectorated or swallowed. Pharmaceutically compatible binding agents or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

In embodiments, a composition of a compound including a nucleic acid sequence of SEQ ID NOs: 1-24 or 128-130 in combination with another pharmaceutically active agent (small molecule or a large biological molecule) formulated for parenteral (including subcutaneous, intramuscular, and intravenous), inhalation, buccal, sublingual, nasal, rectal, topical, or oral administration for treating a viral infection, for inducing immune response, for treating neuroinflammation. The compositions may be conveniently presented in unit dosage form, and prepared by any of the methods well known to one skilled in the art.

In embodiments, the composition of the present disclosure may be formulated in dosage unit form for ease of administration and uniformity of dosage. Dosage unit form as used herein refers to physically discrete units suited as unitary dosages for the individual to be treated; each unit containing a predetermined quantity of agent calculated to produce the desired therapeutic effect in association with the required pharmaceutical carrier. The specification for the dosage unit forms are dictated by and directly dependent on the unique characteristics of the agent and the particular therapeutic effect to be achieved.

### EXAMPLES

The following examples are provided as illustrations of various embodiments of the disclosure but are not meant to limit the disclosure in any manner.

Results discussed herein identify lncMGC and miR-379 as therapeutic targets for the treatment of diabetes, obesity, and complications thereof. For example, miR-379KO mice were protected from not only diabetic kidney disease, but also from chemically-induced type 1 diabetes, muscle atrophy induced by type 1 diabetes, as well as high fat diet induced obesity and kidney injury. Islets from diabetic miR-379KO mice show decreased parameters of ER stress relative to islets from diabetic wild type (WT) control mice, as well as increased insulin.

### Example 1: Testing of GapmeRs Targeting Mouse and Human lncMGC

The microRNA (miRNA)-379 (miR-379), and a mega-cluster (MGC) of microRNAs (including miR-379 and nearly 40 other miRNAs) are involved in cell and mouse models of diabetic nephropathy, a major renal complication of diabetes. See, e.g., Kato et al., Nature Communications, 7, 12864, (2016). These miRNAs induce endoplasmic reticulum (ER) stress, hypertrophy and fibrosis in the kidney. A long non-coding RNA is the host RNA for this cluster of microRNAs (lncMGC). Synthetic antisense GapmeR oligonucleotides modified by locked nucleic acids (LNA) and phosphorothioate (PS) backbone were designed to inhibit lncMGC (lncMGC GapmeRs). Out of several designed GapmeRs, one (MGC10) against mouse lncMGC inhibited the expression of lncMGC and several cluster microRNAs (including miR-379) in cultured mouse kidney mesangial cells in vitro, as well as in mouse kidney cortex in vivo. In parallel, the expression of the microRNA target genes was increased, whereas the expression of profibrotic genes (which promote diabetic nephropathy) was inhibited in vitro and in vivo in mice. Hypertrophy of mouse mesangial cells and mouse kidney glomeruli (features of diabetic nephropathy) were significantly attenuated in diabetic mice.

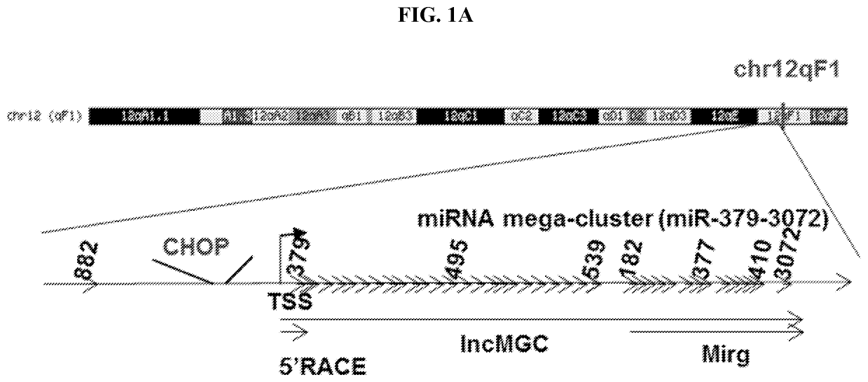

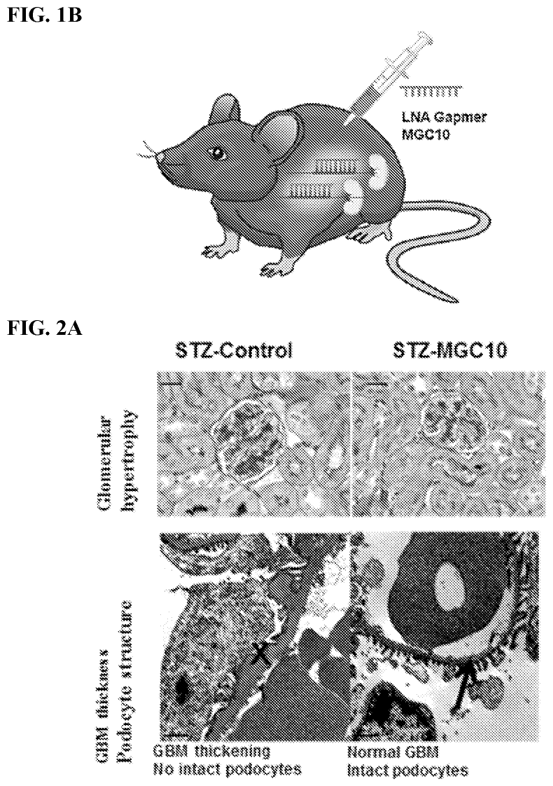

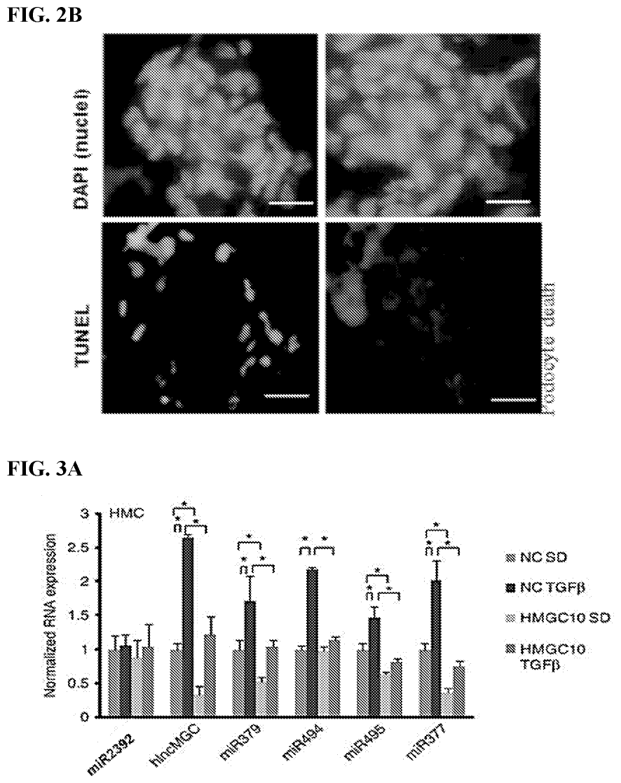

To test the effect of MGC10 in vivo, mice were first made diabetic by injection with streptozotocin (STZ). Some of the STZ-injected Type 1 diabetic mice were injected with MGC10, as illustrated schematically in FIG. 1B. Illustrative images of cells from mice treated with MGC10 (STZ-MGC10) and those not treated with MGC10 (STZ-control) are shown in FIGS. 2A-B. Results indicate that targeting lncRNA-MGC with GapmeR MGC10 in vivo in diabetic mice confers renal protection, including reduction in glomerular hypertrophy and fibrosis, glomerular basement membrane (GBM) thickening and podocyte death (Tunel staining).

There is a version of lncMGC in humans. FIG. 1A depicts a schematic illustration of the microRNA-379 region of chromosome 12 at chr12qF1, and a diagram showing the mega cluster of microRNAs (miRNAs) and their upstream promoter region. The label “CHOP” indicates upstream binding sites for the C/EBP homologous protein (CHOP), a transcription factor (TF) associated with the ER and stress response.

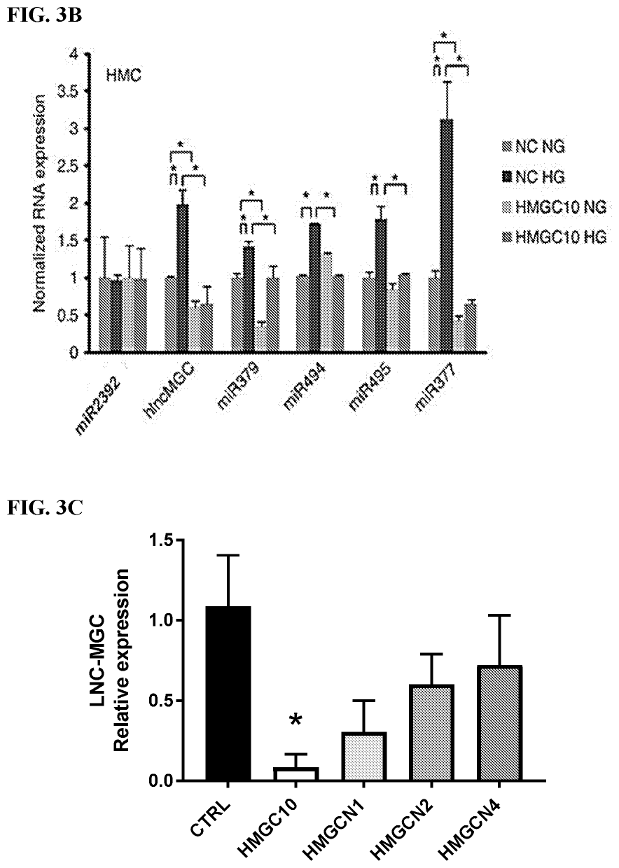

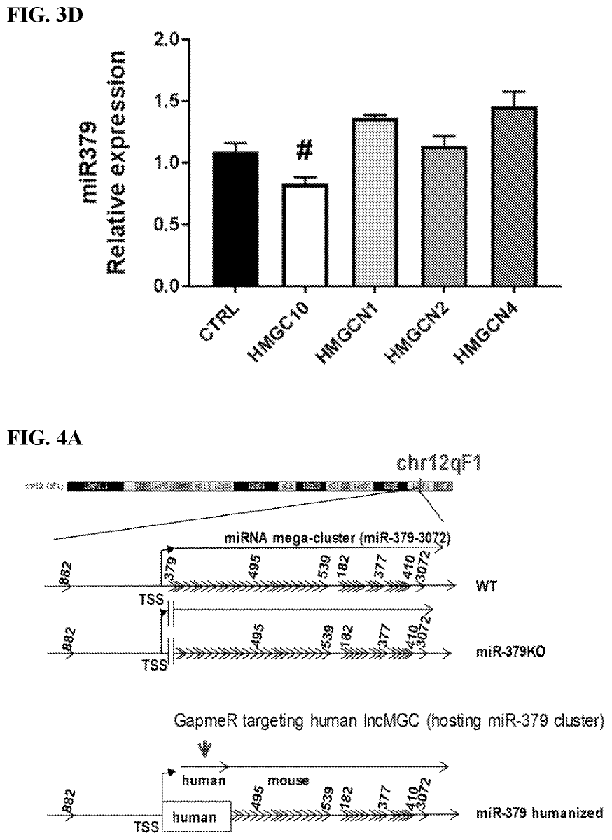

Synthetic oligonucleotides modified by locked nucleic acids (LNA) and phosphorothioate (PS) backbone were designed to inhibit human lncMGC (human lncMGC GapmeRs). Several GapmeRs were tested for their effect on human lncMGC and miR-379 expression levels in Hk-2 cells, a human kidney cell line. Results are illustrated in FIGS. 3C-D. Of the GapmeRs tested, HMGC10 was most effective to reduce the expression of human lncMGC and miR-379 (*, p<0.05). Significant increase of human lncMGC, miR-379, miR-494, miR495, and miR-377 was observed in HMC treated with TGF-β1 (FIG. 3A) or HG (FIG. 3B) relative to respective controls (SD or NG), but not miR-2392 (outside of miR-379 cluster). These increases were significantly reduced in human kidney mesangial cells (HMC) transfected with HMGC10 compared to control oligo. Upregulation of lncMGC and miR-379 by diabetic stimuli like High glucose (HG) or TGF-β was attenuated by HMGC10 in HMC.

### Example 2: Generation of Humanized lncMGC Mice

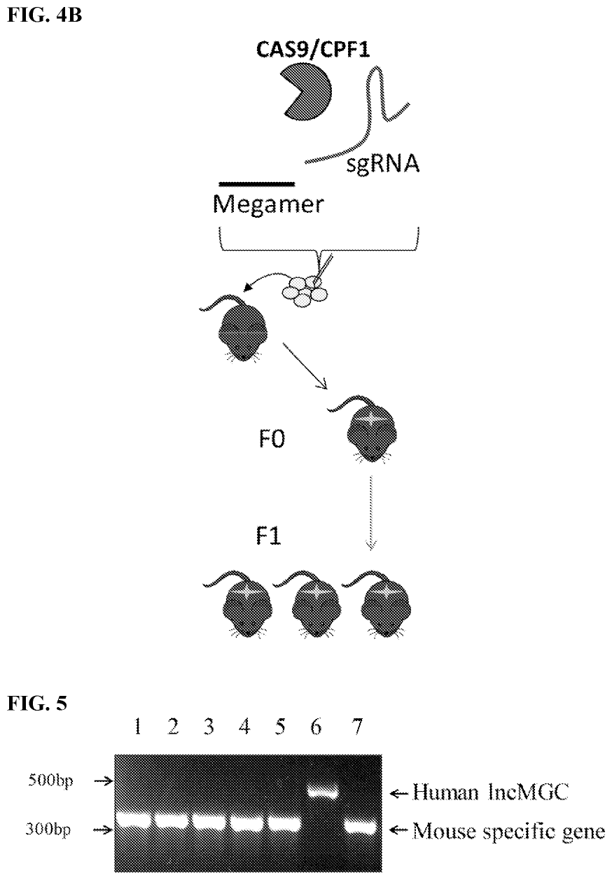

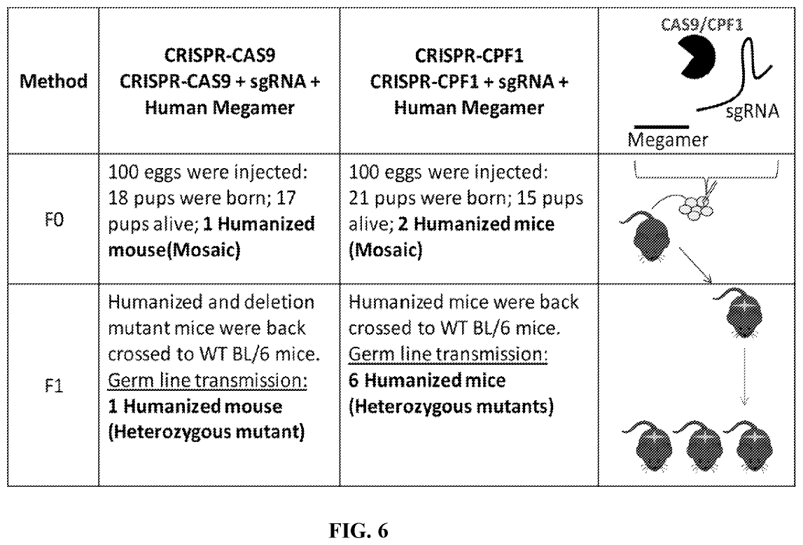

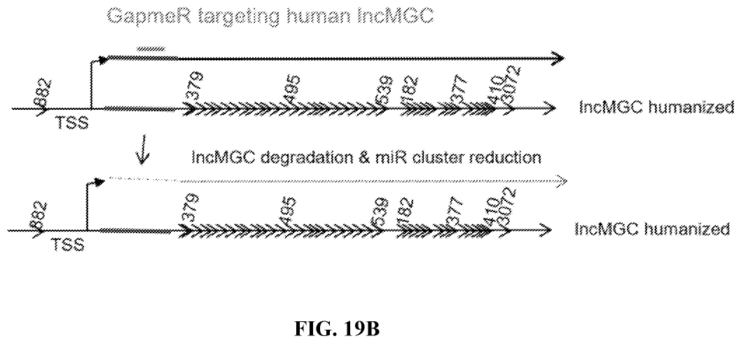

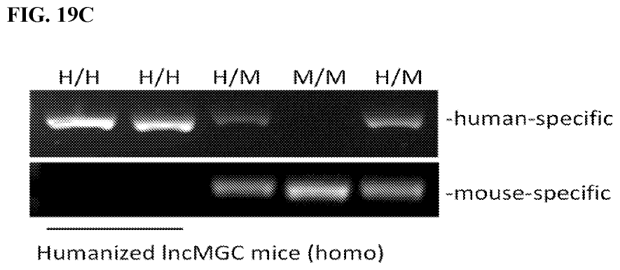

FIG. 4A and FIG. 19A provide schematic diagrams of a strategy for replacing a portion of lncMGC in mice with a corresponding human sequence, thereby creating a humanized lncMGC mouse. FIG. 19A also illustrates a strategy for making a lncMGC knockout (KO) mouse. As illustrated in FIG. 4B, replacement was mediated by CRISPR-Cas9 or CRISPR-Cpf1 genome editing. Resultant mice were backcrossed to obtain mice homozygous for the inserted human sequence. The target region of candidate mice was analyzed by PCR. FIG. 5 illustrates the results of one such PCR analysis. Lane 6, with the longer amplification product, is an F1 mouse resulting from germline transmission of the humanized lncMGC. Another representative PCR analysis of the target region of lncMGC mice is illustrated in FIG. 19C with homozygous genotype for the insertion of humanized lncMGC in 1 lanes and 2, heterogyzous genotype in lanes 3 and 5, and homozygous genotype for mouse lncMGC in lane 4. Additional details regarding the humanization strategy are illustrated in FIG. 6. In both strategies (Cas9 and Cpf1), several humanized lncMGC founders (F0) and germline-transmitted mice (F1) were obtained. Germline-transmitted mice (F1) will be crossed with wild-type mice and a humanized lncMGC mouse colony will be expanded.

The resulting mice comprised human lncMGC GapmeR targets, and were used to test the effects of GapmeRs directed to human lncMGC target sequences (e.g., HMGC10) in mice, such as reducing the incidence of diabetes, obesity, and their complications. FIG. 19B illustrates a strategy for using a GapmeR to target human lncMGC.

### Example 3: In Vivo Protective Effects of miR-379 Knockout

Using the technique of CRISPR-Cas9 genome editing, miR-379 knockout (KO) mice that are deficient in miR-379, which is the first miRNA in the miRNA cluster controlled by lncMGC, were obtained. The strategy for producing the miR-379KO mice is outlined in US20160348105A1 (see, e.g., Example 11).

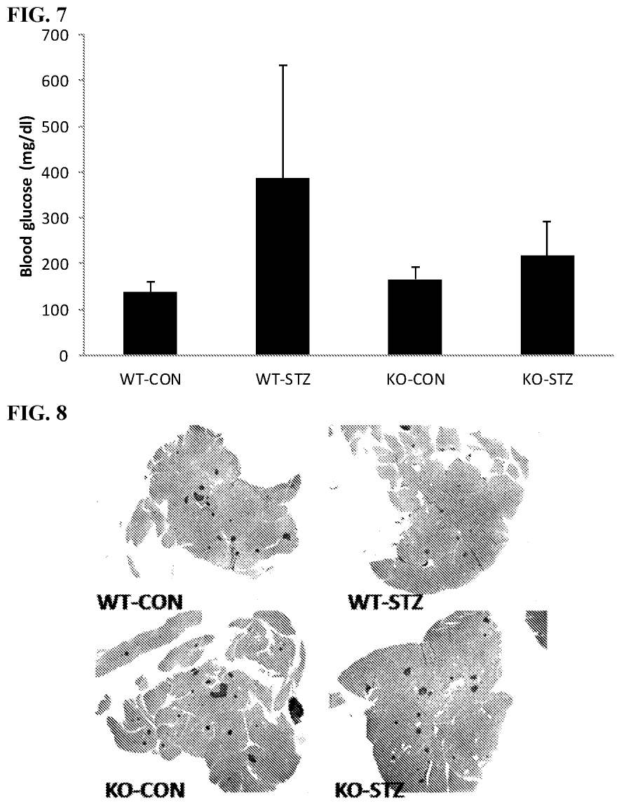

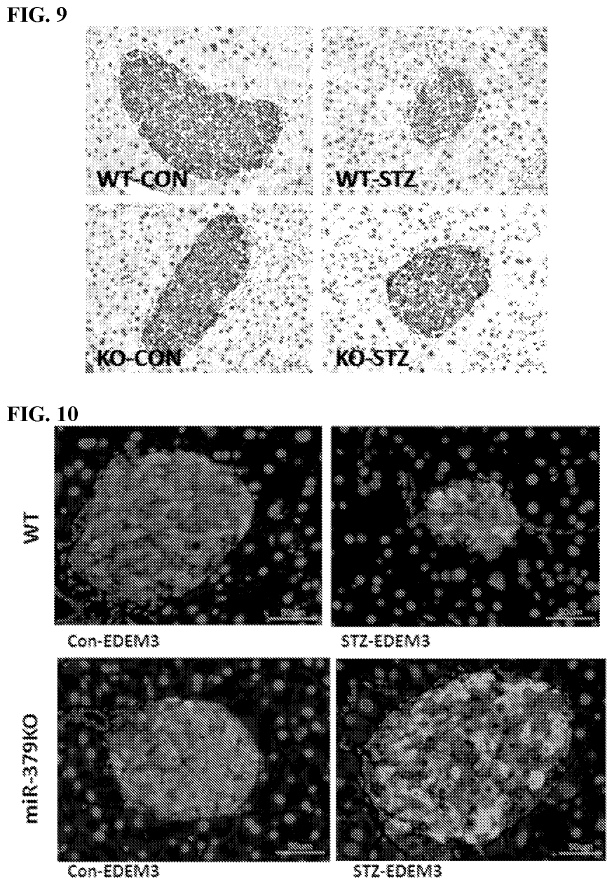

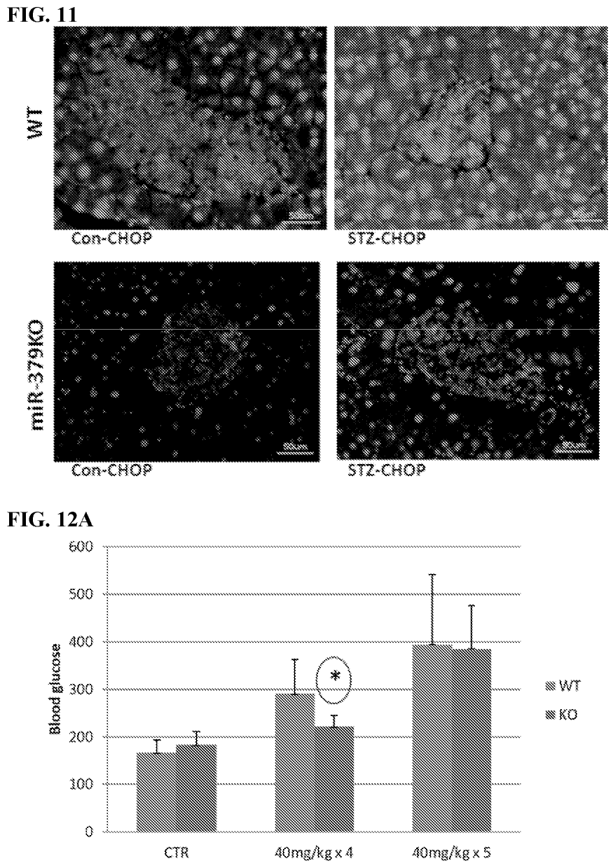

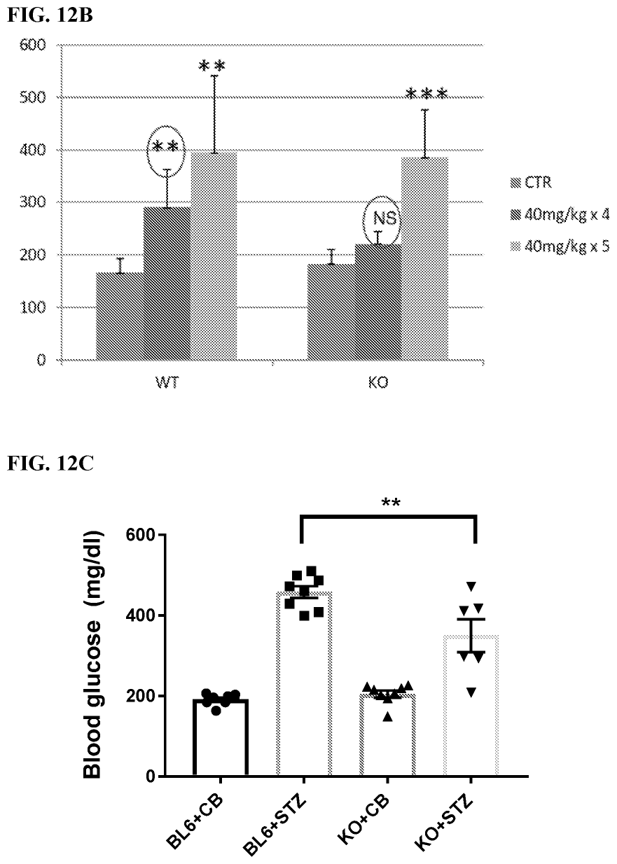

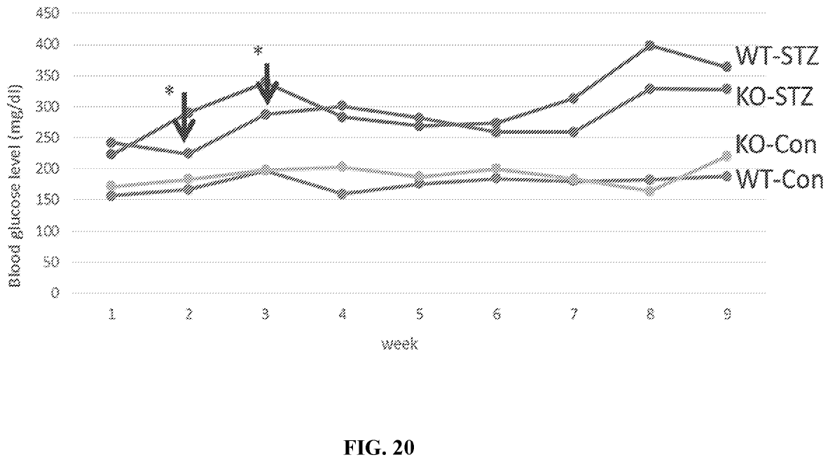

The effects of STZ were evaluated in miR-379KO mice by comparison to wild-type mice. Illustrative results for blood glucose are shown in FIG. 7 and FIG. 20. Mice were evaluated in groups as follows: wild-type mice not treated with STZ (WT-CON), wild-type mice treated with STZ (WT-STZ), knockout mice not treated with STZ (KO-CON), and knockout mice treated with STZ (KO-STZ). Mice treated with STZ were subject to four injections of 40 mg/kg STZ. Blood glucose was lower in STZ-treated miR-379KO mice as compared to STZ-treated wild-type controls. These results indicate miR-379KO mice display delayed onset of hyperglycemia and suggest β-cells in miR-379KO mice are resistant to STZ. As illustrated in FIG. 8, islets were larger and greater in number in miR-379KO treated with STZ as compared to wild-type mice treated with STZ. As illustrated in FIG. 9, there were also more insulin-positive pancreatic β-cells in miR-379KO treated with STZ as compared to wild-type mice treated with STZ. Furthermore, expression of endoplasmic reticulum degradation-enhancing alpha-mannosidase-like 3 (EDEM3) was higher in miR-379KO mice treated with STZ as compared to wild-type mice treated with STZ (FIG. 10). EDEM3 protects against ER stress and is a target of miR-379. In contrast, CHOP expression was lower in miR-379KO mice treated with STZ as compared to wild-type mice treated with STZ (FIG. 11). CHOP increases ER stress and islet dysfunction. Effects on blood glucose were also compared using three different regimens of STZ treatment, four or five injections of STZ at 40 mg/kg or five injections of STZ at 50 mg/kg. Illustrative results are shown in FIGS. 12A-C, and show that blood glucose levels in miR-379KO mice treated with the four STZ injections were lower as compared to wild-type mice treated with the four STZ injection. Similarly, blood glucose levels in miR-379KO mice treated with the five 50 mg/mL STZ injections were lower as compared to wild-type mice treated with the STZ injection. These results identify lncMGC and miR-379 as therapeutic targets for the treatment of diabetes, such as type 1 diabetes.

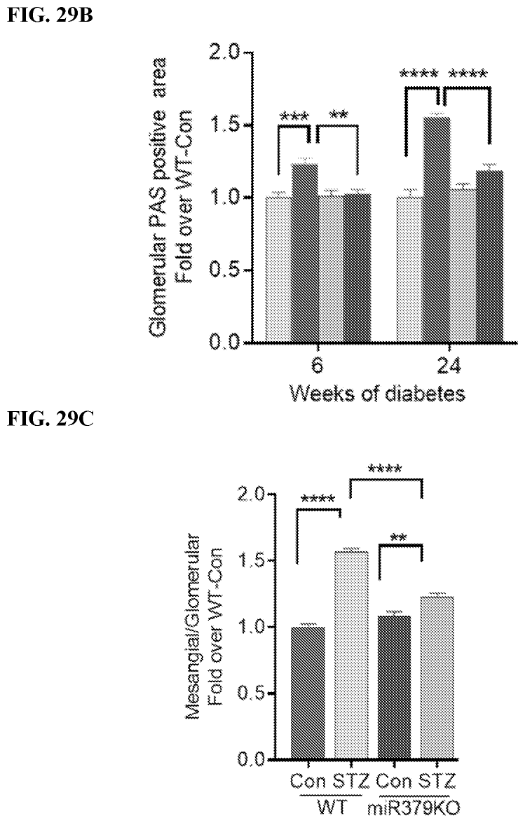

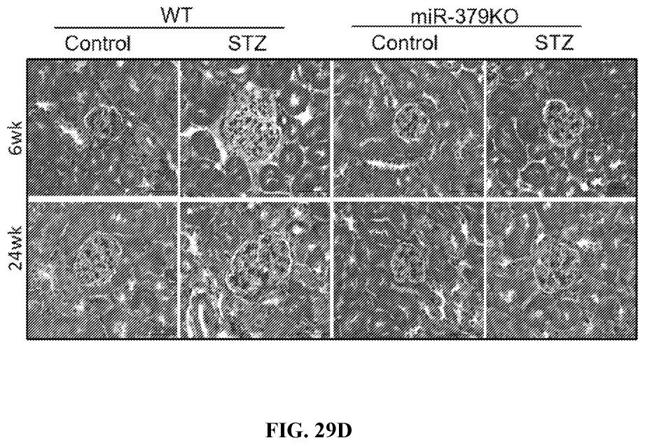

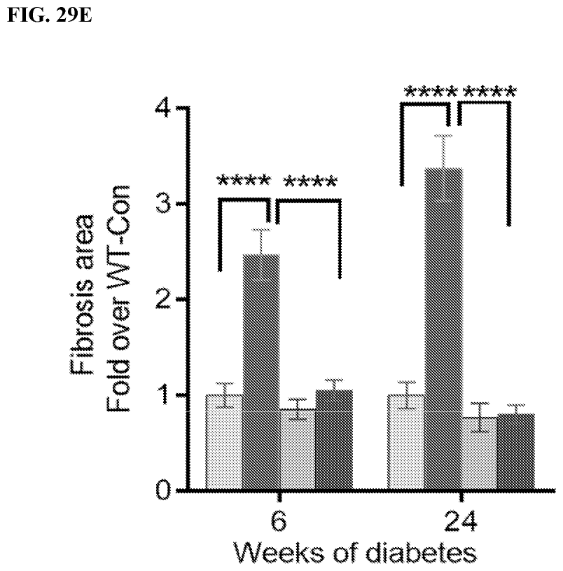

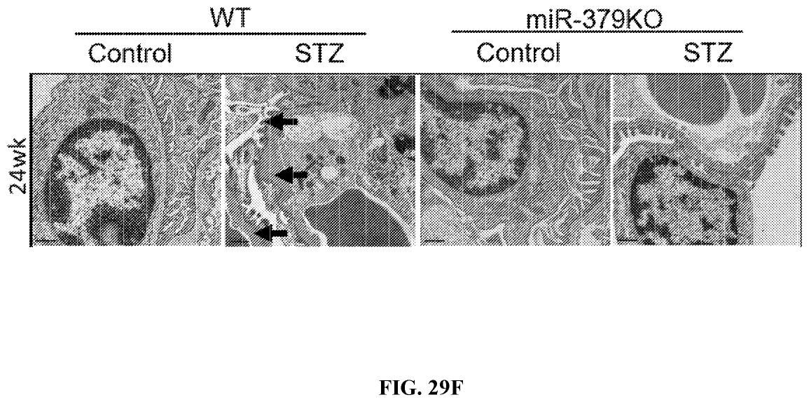

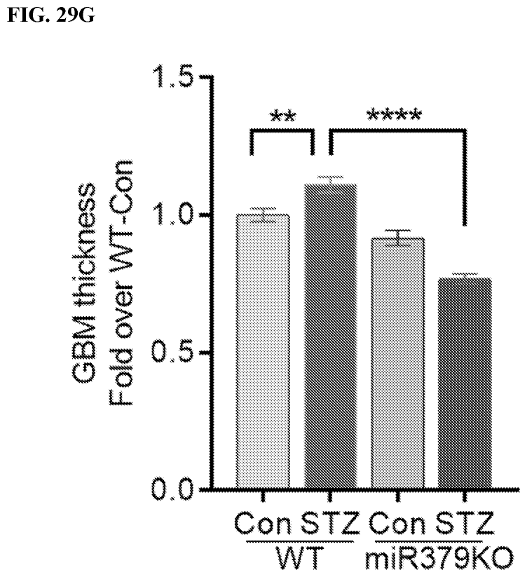

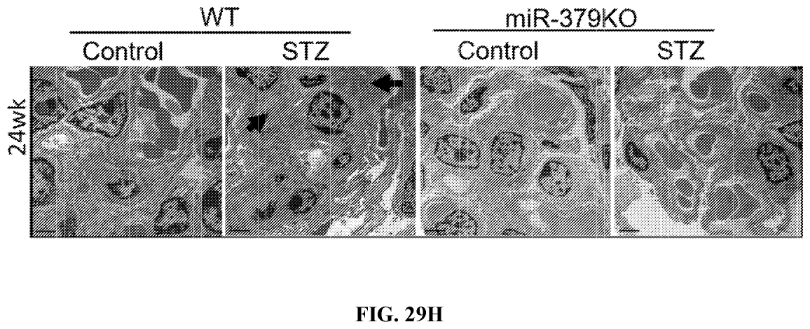

Effects of STZ were evaluated in miR-379KO mice as compared to WT mice. Mice were grouped as follows: Non-diabetic WT control (WT-Con), diabetic wild type (WT-STZ), non-diabetic miR-379 KO control (miR379KO-Con), and diabetic miR-379 KO (miR379KO-STZ). As illustrated in FIGS. 29A-G, WT-STZ mice showed significant increases in mesangial matrix expansion, mesangial expansion, and glomerular fibrosis compared to the non-diabetic controls; in contrast, the increases in mesangial matrix expansion, mesangial expansion, and glomerular fibrosis were ameliorated in miR-379KO-STZ mice at 6 or 24 weeks following diabetes onset. WT-Con showed uniformly thin glomerular basement membranes (GBM) and normal structures of podocytes and foot processes; in contrast, WT-STZ mice exhibited thickening of the GBM and effacement of podocyte foot processes. Although non-diabetic miR-379KO mice exhibited normal structures, similar to WT control mice, the GBM thickening and podocyte foot process effacement observed in WT-STZ mice were attenuated in miR-379KO-STZ mice. Quantitative analysis confirmed that the GBM was significantly thinner in miR-379KO-STZ mice than in WT-STZ mice. Furthermore, WT-STZ mice developed excessive mesangial expansion with electron-dense deposits, which was attenuated in miR-379KO-STZ mice at 24 weeks after diabetes onset. These results indicate that diabetic miR-379KO-STZ mice experience reduced severity in key features of DKD, and suggest that miR-379KO mice are protected from DKD. Therefore, targeting lncMGC with a GapmeR as described herein is expected to exhibit likewise protective effects.

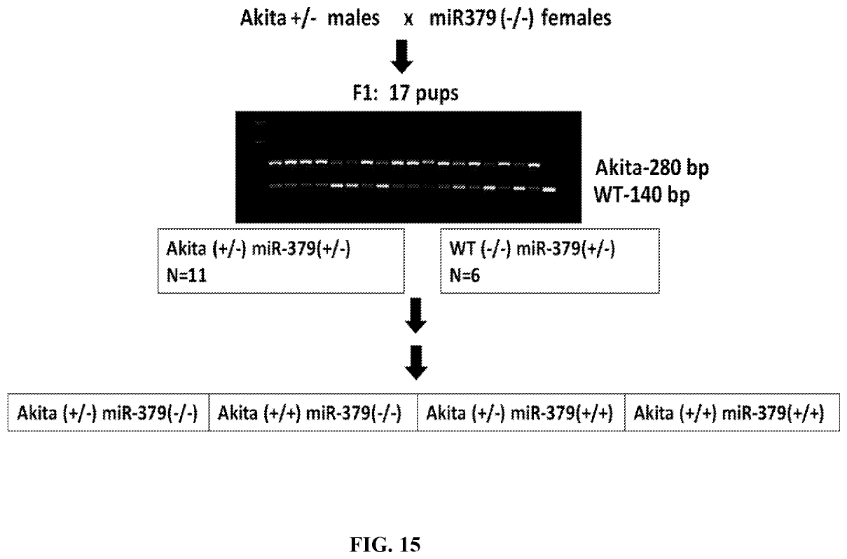

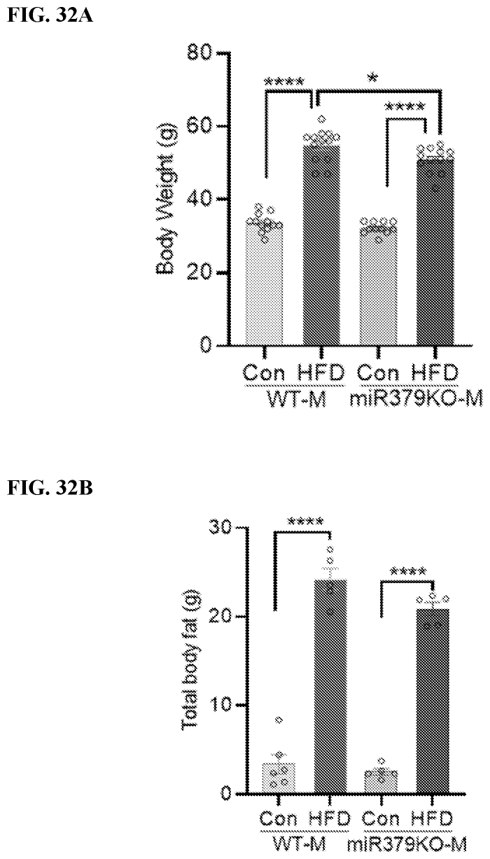

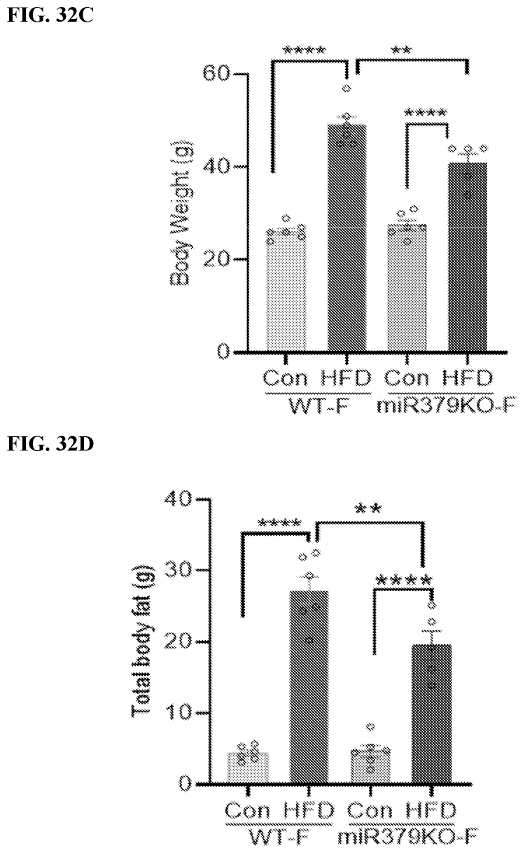

The effects of miR-379 knockout on body weight in mice fed a high-fat diet (HFD) was evaluated. Male mice were grouped as follows: wild-type mice fed a control diet (WT-Con-Male), wild-type mice fed a high-fat diet (WT-HFD-Male), miR-379KO mice fed a control diet (KO-Con-Male), and miR-379KO mice fed a high-fat diet (KO-HFD-Male). Female mice were grouped as follows: wild-type mice fed a control diet (WT-Con-F), wild-type mice fed a high-fat diet (WT-HFD-F), miR-379KO mice fed a control diet (KO-Con-F), and miR-379KO mice fed a high-fat diet (KO-HFD-F). Body weight was followed over time. Illustrative results are shown in FIG. 14 and FIGS. 32A-D, showing statistically significant lower body weight among the miR-379KO mice on HFD as compared to wild-type mice on HFD. The results identify lncMGC and miR-379 as therapeutic targets for anti-obesity therapy.

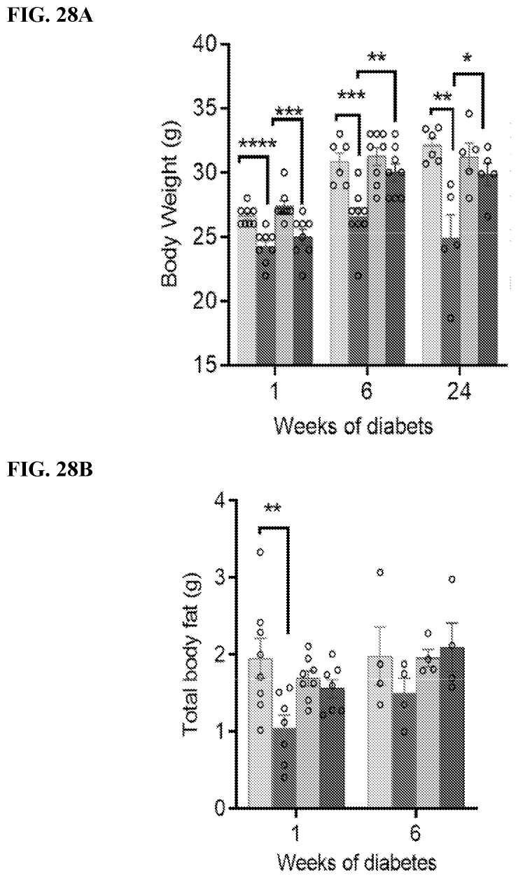

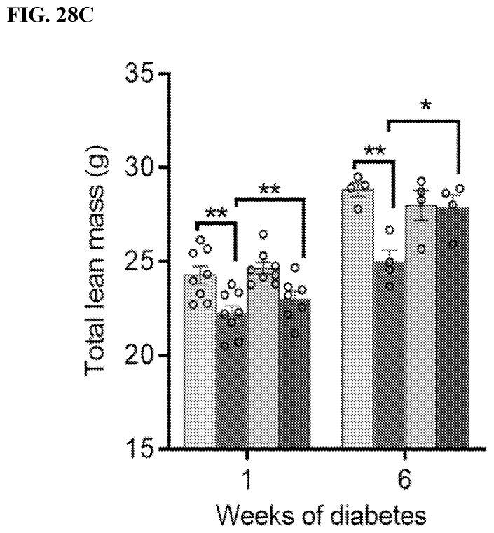

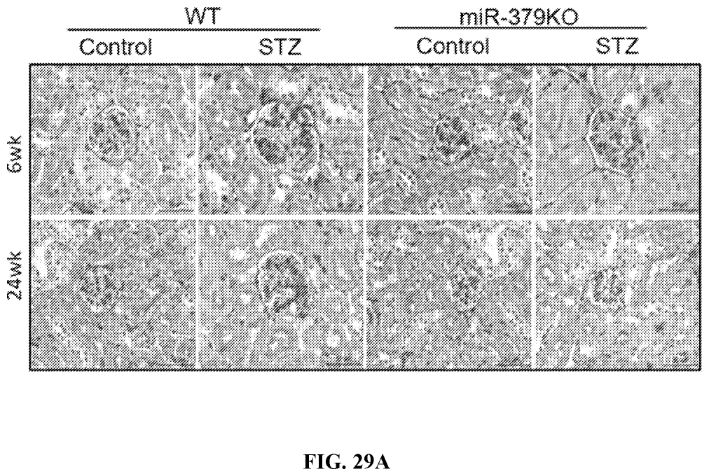

The effect of miR-379 knockout on body weight in STZ-induced diabetic mice was evaluated. FIGS. 28A-C illustrate significant reduction of body weight, as measured in fat and lean mass by body composition analysis using Echo/MRI systems in diabetic WT mice. Body weight was restored in STZ diabetic miR-379KO mice. The results identify lncMGC and miR-379 as therapeutic targets for inhibiting weight loss for treating diabetes related complications.

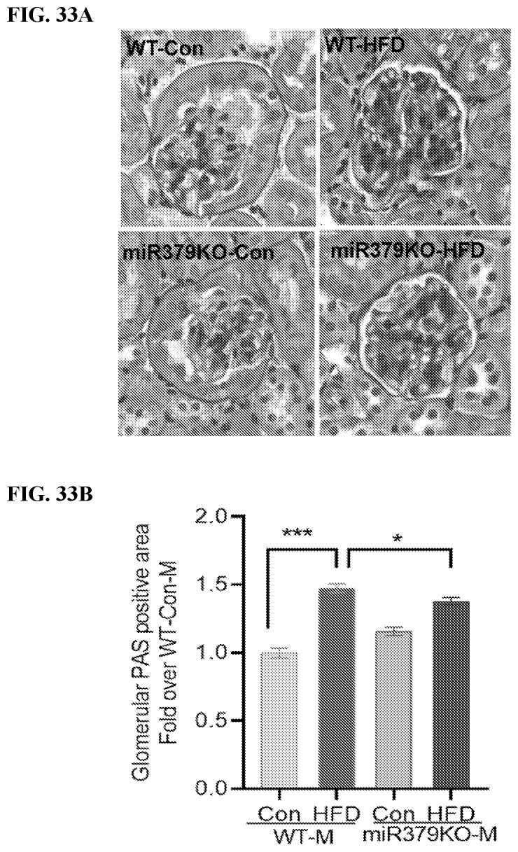

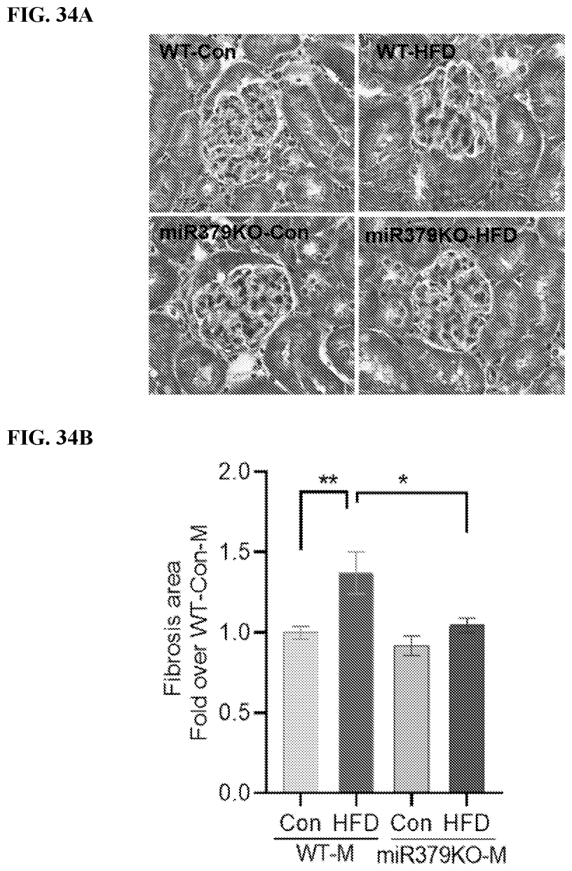

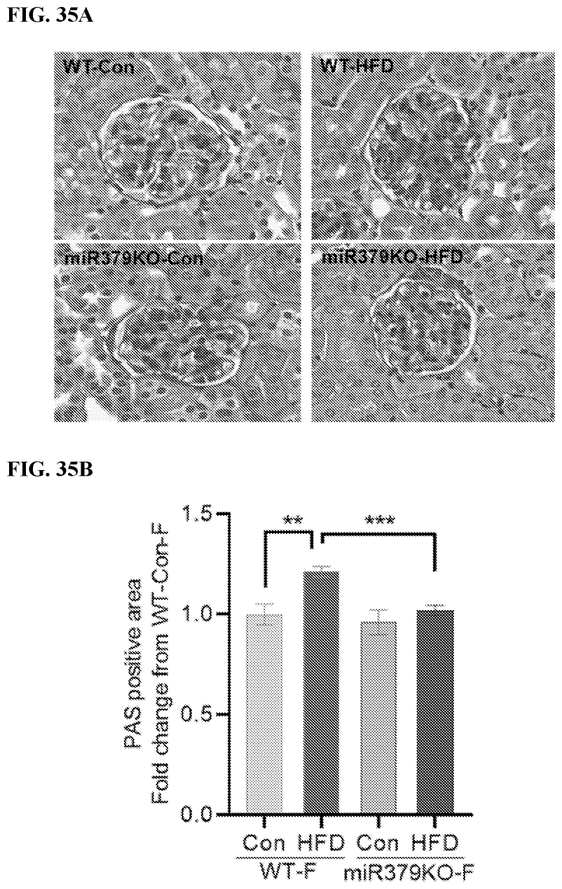

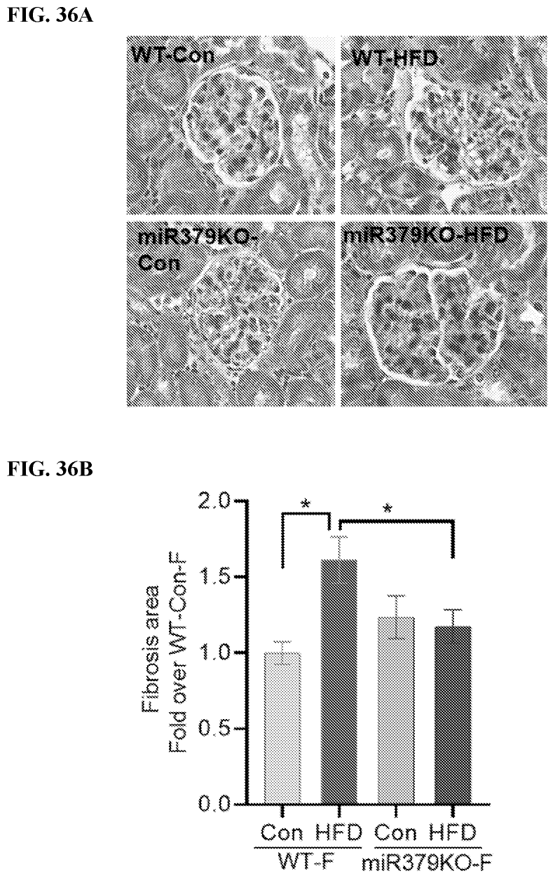

The effects of miR-379 on glomerular tissue in mice fed a high-fat diet (HFD) was evaluated. WT Mice fed HFD display increased glomerular mesangial area and extracellular-matrix (ECM) accumulation, which are attenuated in miR-379KO male and female mice, as illustrated in FIGS. 33A-B and FIGS. 35A-B. Following 24 weeks on HFD, Masson's trichrome staining of glomerular tissue show fibrosis among the WT mice, while the miR-379KO show statistically significant lower fibrosis area. Illustrative results shown in FIGS. 34A-B and FIGS. 36A-B. As such, miR-379 is a viable target for protecting glomerular tissue in instances of kidney injury, such as kidney injury incident to a high-fat diet.

The miR-379KO mice were crossed with a genetic mouse model of type 1 diabetes (Akita mice) to further evaluate the effects of GapmeRs targeting lncMGC. An example process for crossing with Akita diabetic mice is illustrated in FIG. 15, which includes a gel image for a PCR analysis of F1 pups to identify those heterozygous for the Akita genotype. Mice produced from the cross will be compared with regard to blood glucose, body weight, kidney functions, and other complications. In view of the protective effects conferred by miR-379KO in the chemically-induced model of type 1 diabetes, it is expected that mice from the cross having the miR-379KO will also exhibit protective effects as compared to the Akita parental line. GapmeRs (e.g., MGC10) can be administered to Akita mice, and it is expected that such administration will also treat the diabetic phenotype. Mice homozygous for miR-379KO are also expected to be protected against incidence of diabetes, obesity, and complications thereof.

### Example 4: Additional Mouse Experiments

The miR-379KO mice are crossed with other mouse models of disease to evaluate the therapeutic effects of humanized GapmeRs (e.g., HMGC10). Non-limiting examples of such mouse models include NOD, db/db (type 2 diabetes), and ob/ob (obesity).

GapmeRs targeting lncMGC are administered in mouse models of human disease to evaluate the therapeutic effects on the respective conditions. Mouse models can be genetic mouse models, such as those discussed above, or a disease state can be induced. Examples of induced disease states include STZ-induced diabetes, and HFD-induced obesity.

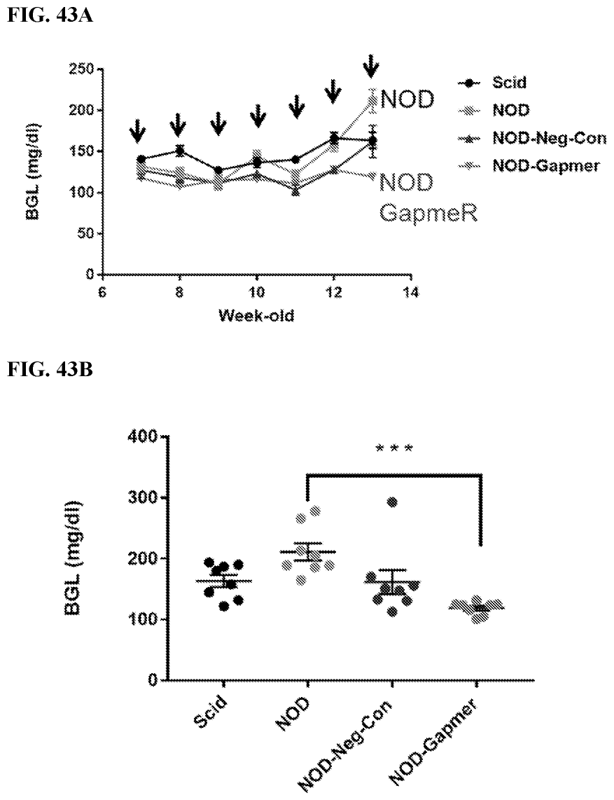

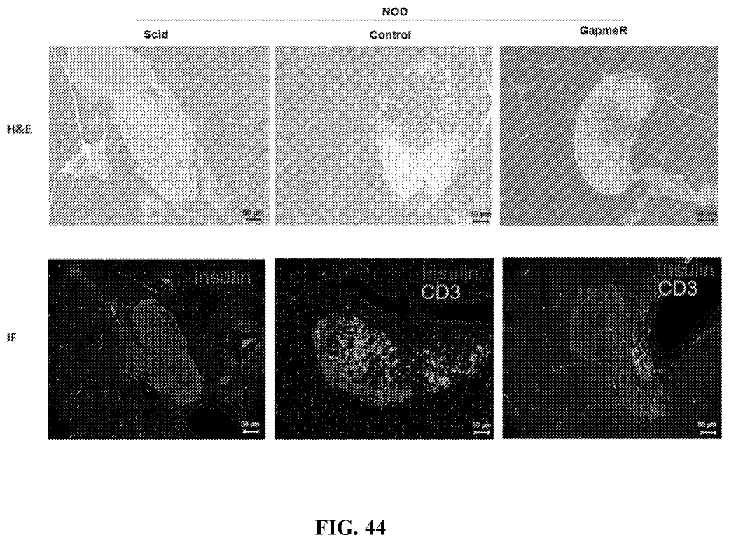

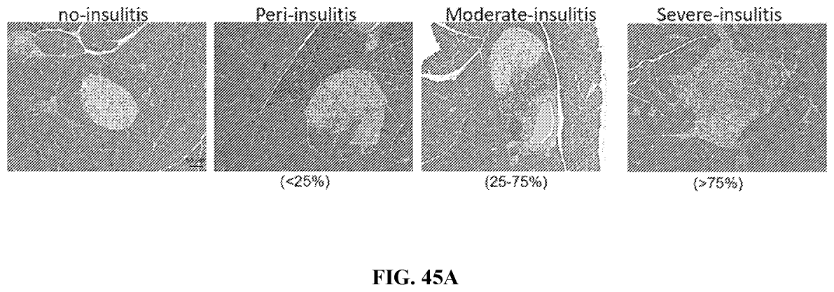

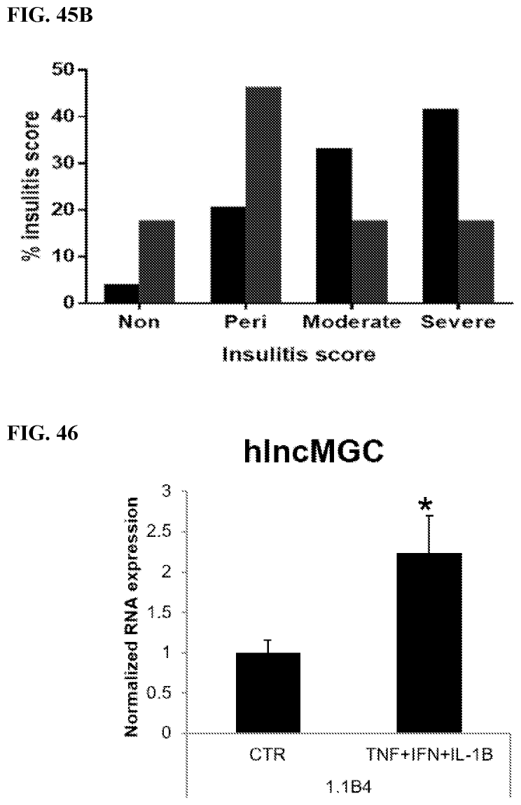

Effects of GapmeR lncMGC were evaluated in genetic type 1 diabetes model NOD mice. Illustrative effects of GapmeR lncMGC on blood glucose are shown in FIGS. 43A-B. Mice were evaluated in groups as follows: Scid mice, NOD mice, NOD-Neg-Con, and NOD-Gapmer. The NOD-Gapmer mice were injected with 5 mg/mkg GapmeR lncMGC weekly, and showed statistically significant lower blood glucose levels as compared to NOD and NOD-Neg-Con mice. Further, NOD mice treated or untreated with GapmeR lncMGC were evaluated for insulitis, and were grouped as follows: Scid, Control, and GapmeR. Illustrative results for attenuation of insulitis by GapmeR lncMGC are shown in FIG. 44 and FIGS. 45A-B. NOD mice were treated with GapmeR lncMGC at a weekly dosage of 5 mg/kg mouse, and Control and Scid mice received no GapmeR lncMGC. Images in FIG. 44 show tissue stained for insulin and CD3 to detect insulitis. NOD mice injected with GapmeR lncMGC show presence of insulin and low infiltration of CD3 positive cells, indicating attenuated insulitis. Further, NOD mice treated with GapmeR lncMGC showed a significantly lower insulitis score, as shown in FIG. 45B. These results identify GapmeR lncMGC as a viable therapeutic for preventing type 1 diabetes.

Mice that are homozygous for a knockout of lncMGC are created by a process similar to that used for producing the miR-379KO mice. The lncMGC-KO mice are compared to wild-type mice for protection against incidence of diabetes, obesity, and complications thereof. Considering the protective effect of miR-379KO, lncMGC-KO is also expected to be protective.

### Example 5: In Vivo Protective Effects of miR-379 Poly-A Knock-in

CRISPR-Cas9 editing was used to create mice having a poly-A knock-in (KI) at the miR-379 position, terminating transcription of the whole miRNA cluster and lncMGC. Results similar to those for the miR-379KO mice were observed for the miR-379KI mice, including protection from diabetes, obesity, and complications. Results from these two mouse models indicate that suppression of the lncMGC could be applied to treat diabetes, obesity, and complications.

### Example 6: Increased Levels of lncMGC in Type 2 Diabetes

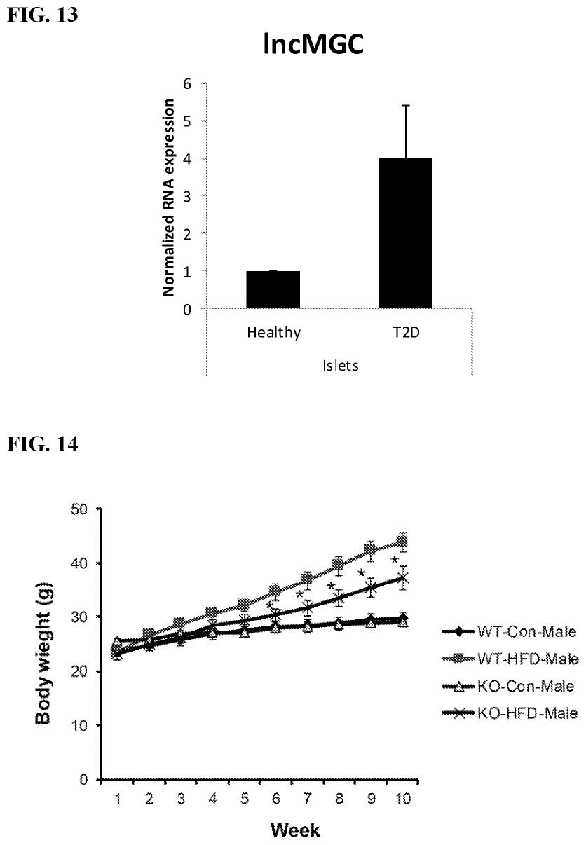

Human islets were isolated from type 2 diabetes patients, and the level of lncMGC expression was measured. These expression levels were compared to those of healthy controls. Illustrative results are shown in FIG. 13, showing significantly increased lncMGC expression levels in type 2 diabetics (T2D) as compared to controls (Healthy). These results identify lncMGC as a therapeutic target for the treatment of diabetes, such as type 2 diabetes.

### Example 7: Identification of Mettl3 as Target of lncMGC miRNAs

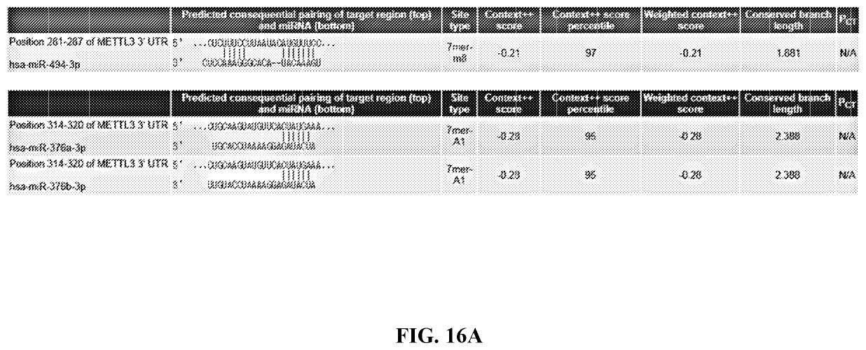

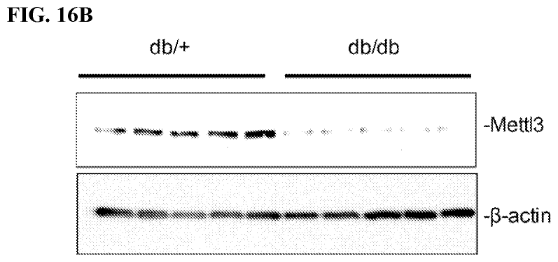

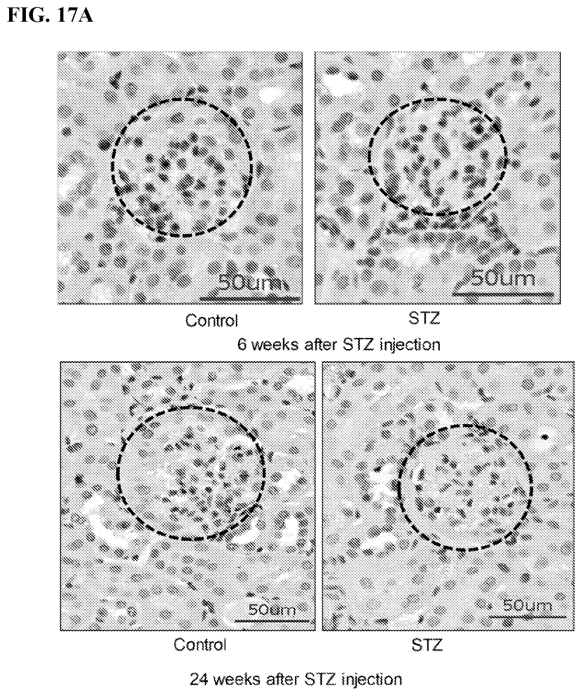

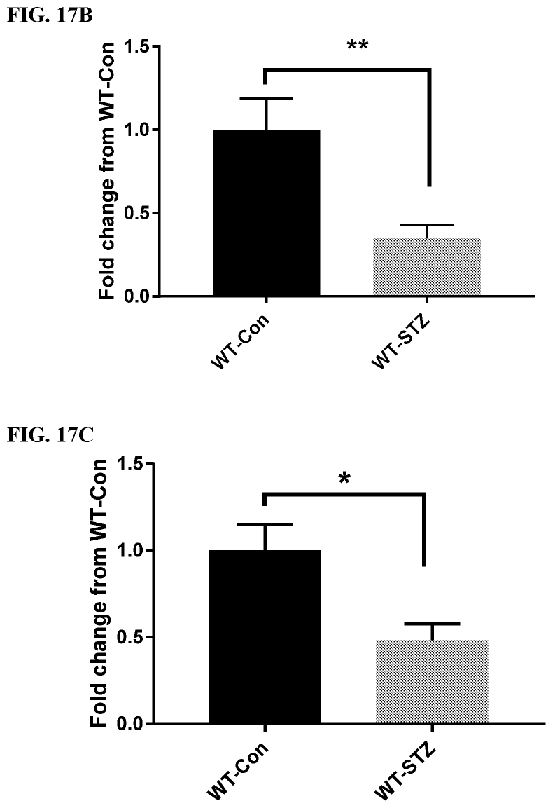

FIG. 16A illustrates predicted target sites for miR-494 (top table) and miR-376 (bottom table) in the 3′ UTR of Mettl3. Both of the miRNAs are members of the miR-379 cluster. Consistent with these predictions, expression of Mettl3 in glomeruli is reduced in both db/db mice relative to db/+ mice (FIG. 16B), and in STZ-induced diabetic mice relative to untreated mice (FIGS. 17A-C). Mettl3 is an important component of the RNA methyltransferase complex, which can affect gene expression. These results indicate that GapmeRs targeting lncMGC can control the miRNA cluster and regulate pathologic cellular signaling and events, and are useful to treat diabetes, obesity, and related complications.

### Example 8: Identification of miR-379 Targets

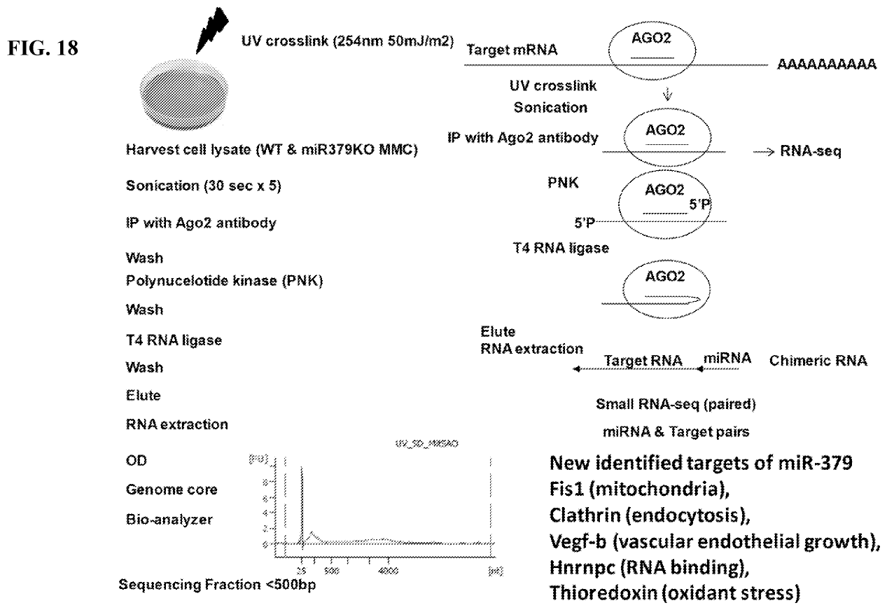

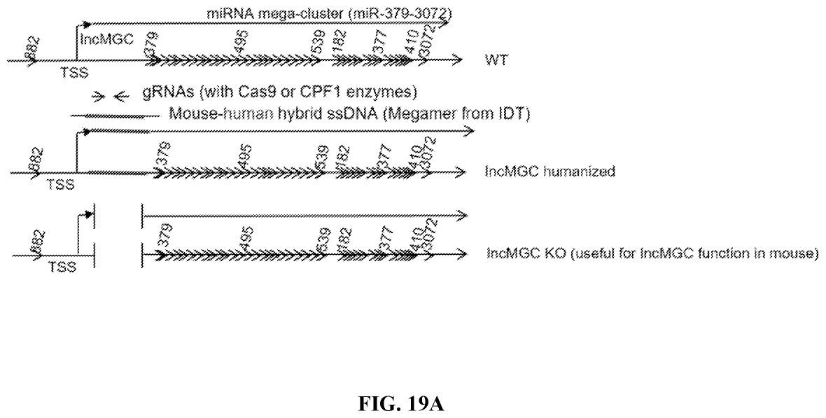

Targets of miR-379 were identified using a process involving crosslinking, ligation, and sequencing of hybrids (CLASH). FIG. 18 provides a diagram of an illustrative CLASH process. Mouse kidney mesangial cells (MMC) derived from miR-379KO mice and MMC derived from wild-type mice were compared. UV cross-linked RNA-protein complexes from these cells were sonicated, immunoprecipitated with Ago2 antibody, and ligated. Hybrid RNAs were subjected to RNA sequencing. Candidate miR-379 targets identified by Ago2-IP RNA-seq and ranked by significant decrease of enrichment in Ago2-IP in miR-379KO MMC compared to WT MMC are shown in Table 4. Candidate miR-379 targets identified by Ago2-IP RNA-seq and ranked by enrichment in Ago2-IP in WT MMC are shown in Table 5. Several targets were identified, including Fis1 (related to mitochondrial fission), Clathrin (endocytosis), Vegf-β (vascular endothelial cell growth), thioredoxin (oxidant stress), EDEM3 (ER stress regulator), and Hnrnpc (RNA binding).

TABLE 4
Candidate miR-379 targets ranked by significant decrease of
enrichment in Ago2-IP in miR-379KO MMC compared to WT MMC

| | log2.tar- | log2.tar- | log2.tar- |
|---|---|---|---|
| | get.vs.3UTR.WTA_IP.tar- | get.vs.3UTR.WT8_IP.tar- | get.vs.3UTR.X379KOA_IP.tar- |
| gene_symbol | get.cov | get.cov | get.cov |
| Clta | 2.003924 | 1.701273 | −0.12614 |
| Fis1 | 1.282478 | 1.208693 | −0.21804 |
| Vegfb | 2.276106 | 2.044893 | 0.902405 |
| Ap3s1 | 2.080585 | 2.450011 | 1.492658 |
| Hnrnpc | 1.412723 | 1.726675 | 1.081704 |
| Txn1 | 3.580309 | 2.627671 | 2.869199 |
| Slc20a1 | 1.739989 | 1.648513 | 0.906599 |

| | log2.tar- | | | | |
|---|---|---|---|---|---|
| | get.vs.3UTR.X379KOB_IP.tar- | | | | fold |
| gene_symbol | get.cov | WT | KO | WT − KO | change |
| Clta | −0.14946 | 1.852598 | −0.1378 | 1.990397 | 3.973463 |
| Fis1 | 0.342257 | 1.245586 | 0.062106 | 1.183479 | 2.271238 |
| Vegfb | 1.247112 | 2.1605 | 1.074759 | 1.085741 | 2.122466 |
| Ap3s1 | 1.315009 | 2.265298 | 1.403833 | 0.861465 | 1.816882 |
| Hnrnpc | 1.04241 | 1.569699 | 1.062057 | 0.507642 | 1.421725 |
| Txn1 | 2.538936 | 3.10399 | 2.704067 | 0.399923 | 1.319438 |
| Slc20a1 | 2.008699 | 1.694251 | 1.457649 | 0.236602 | 1.178214 |

TABLE 5
Candidate miR-379 targets ranked by enrichment in Ago2-IP in WT MMC

| | log2.tar- | log2.tar- | log2.tar- |
|---|---|---|---|
| | get.vs.3UTR.WTA_IP.tar- | get.vs.3UTR.WTB_IP.tar- | get.vs.3UTR.X379KOA_IP.tar- |
| gene_Symbol′ | get.cov | get.cov | get.cov |
| Txn1 | 3.580309 | 2.627671 | 2.869199 |
| Ap3s1 | 2.080585 | 2.450011 | 1.492658 |
| Vegfb | 2.276106 | 2.044893 | 0.902405 |
| Clta | 2.003924 | 1.701273 | −0.12614 |
| Slc20a1 | 1.739989 | 1.648513 | 0.906599 |
| Hnrnpc | 1.412723 | 1.726675 | 1.081704 |
| Fis1 | 1.282478 | 1.208693 | −0.21804 |

| | log2.tar- | | | | |
|---|---|---|---|---|---|
| | get.vs.3UTR.X379KOB_IP.tar- | | | | fold |
| gene_Symbol′ | get.cov | WT | KO | WT − K | change |
| Txn1 | 2.538936 | 3.10399 | 2.704067 | 0.399923 | 1.319438 |
| Ap3s1 | 1.315009 | 2.265298 | 1.403833 | 0.861465 | 1.816882 |
| Vegfb | 1.247112 | 2.1605 | 1.074759 | 1.085741 | 2.122466 |
| Clta | −0.14946 | 1.852598 | −0.1378 | 1.990397 | 3.973463 |
| Slc20a1 | 2.008699 | 1.694251 | 1.457649 | 0.236602 | 1.178214 |
| Hnrnpc | 1.04241 | 1.569699 | 1.062057 | 0.507642 | 1.421725 |
| Fis1 | 0.342257 | 1.245586 | 0.062106 | 1.183479 | 2.271238 |

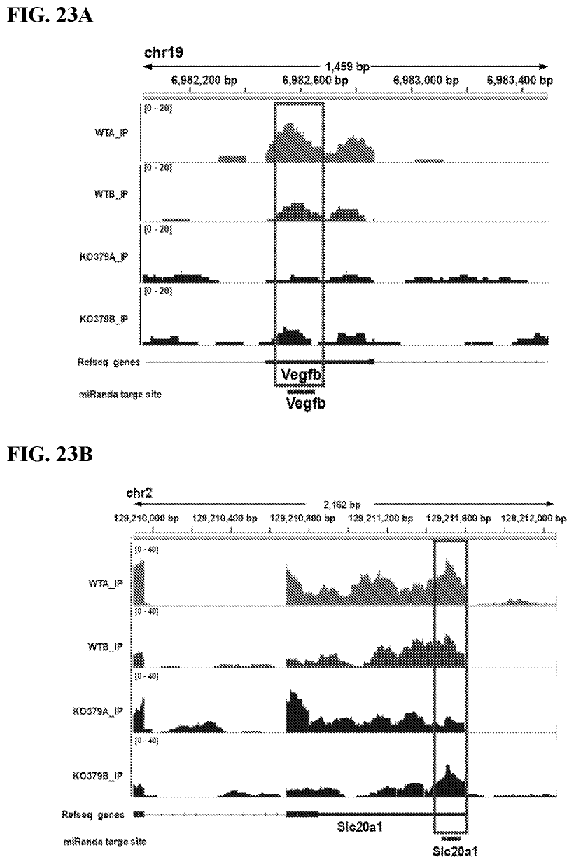

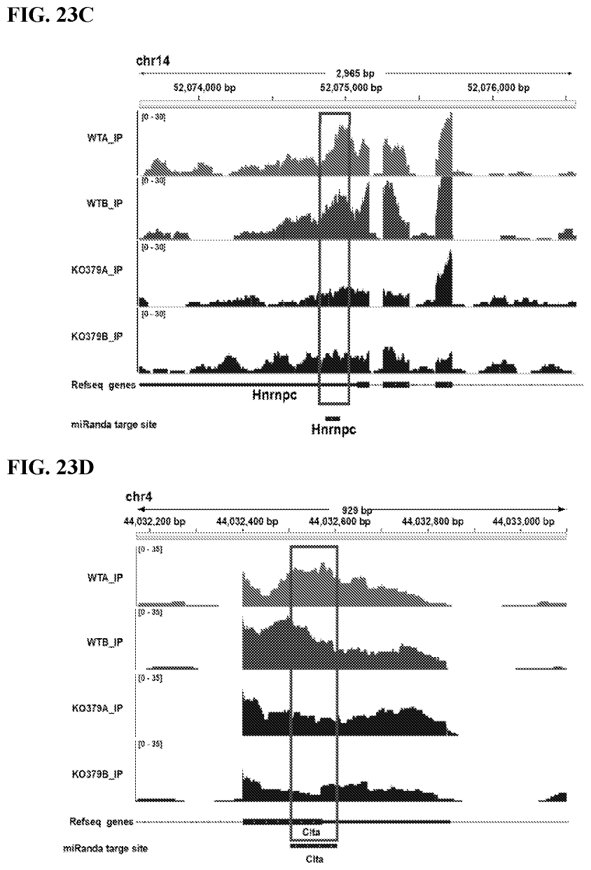

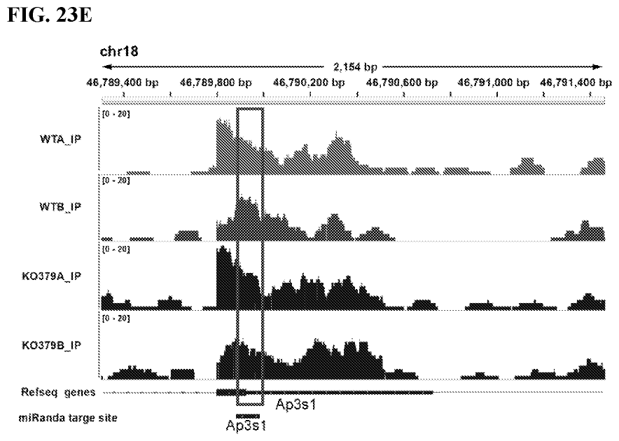

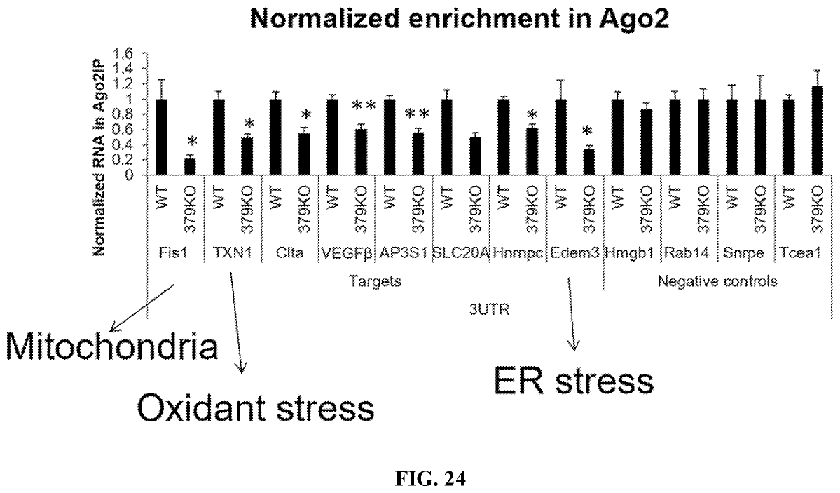

Illustrative results in FIGS. 23A-E display enrichment of RNA reads at the miR-379 target site in miR-379KO MMC and WT MMC. Several miR-379 target candidates were identified, including Vegfb, Slc20a1, Hnrnpc, Clta and, Ap3s1, all of which show significant reduction in RNA reads in miR-379KO MMC compared to WT MMC. Enriched candidates identified by Ago2-IP-seq were subject to qPCR validation, as shown in FIG. 24. In all seven candidate miR-379 targets (EDEM3, Fis1, Txn1, Vegfb, Slc20a1, Hnrnpc, Clta and Ap3s1), decreased RNA levels were observed in miR-379KO cells. Rab14, Snrpe, Tcea1, and Hmgb1 were used as stoic (negative) controls because no significant change between miR-379KO and WT MMC was detected in both RNA-seq and qPCR analyses.

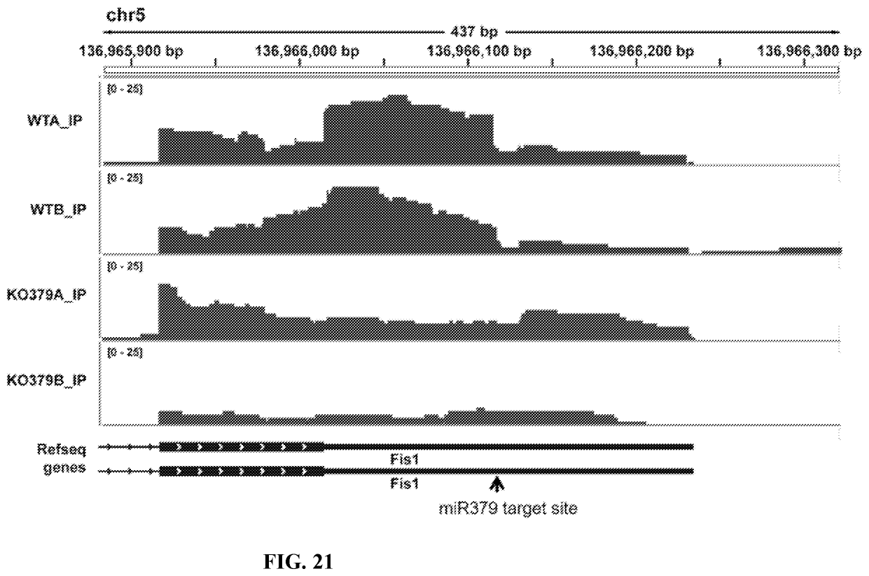

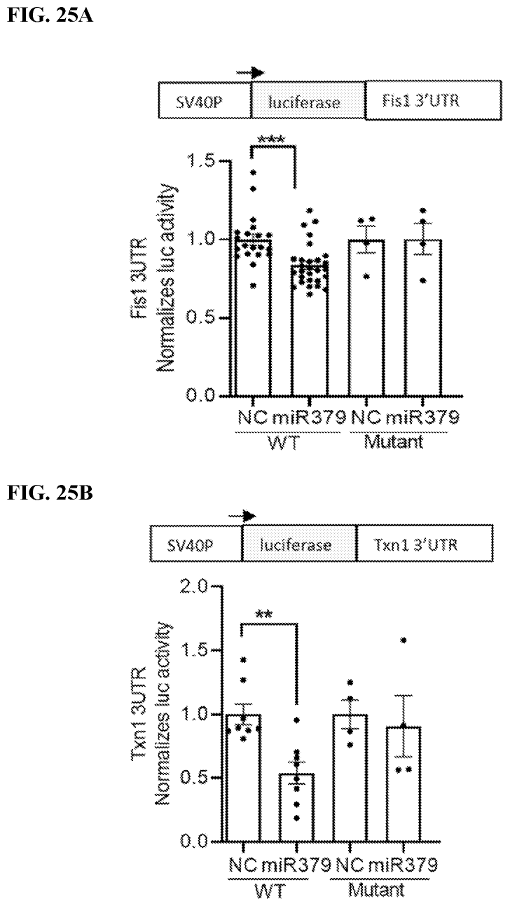

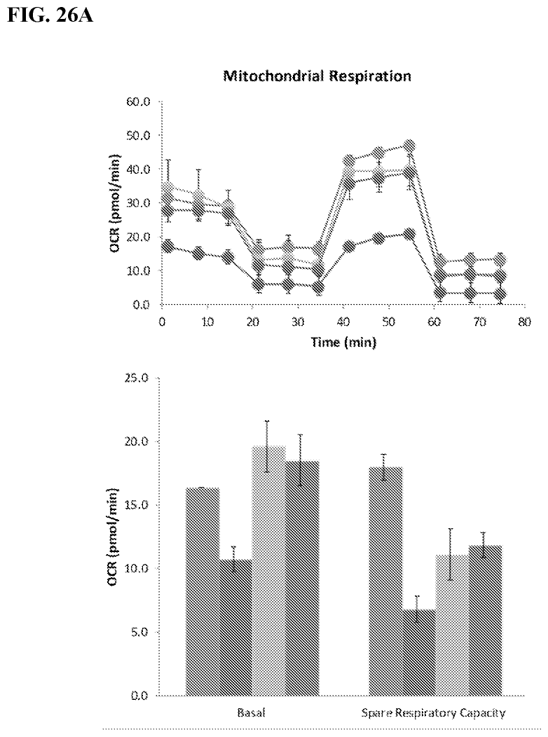

Fis1 was confirmed as a bona fide miR-379 target by Ago2 qPCR and Fis1 3′UTR reporter assays. FIG. 21 depicts enrichment of RNA reads at miR-379 target site Fis1 3′UTR in WT mouse mesangial cells (MMC), with notable reduction in miR-379KO MMC. Further, as illustrated in FIG. 25A, there was a significant decrease in activity in the luciferase-Fis1 3′UTR reporter by miR-379, compared to insignificant change in activity in the mutant Fis1 3′UTR reporter, which abolishes miR-379 binding, indicating Fis1 3′UTR is a true target of miR-379.

siRNA-mediated Fis1 knockdown reduced key mitochondrial signals in MC, suggesting Fis1 is involved in mitochondrial dysfunction in diabetic neuropathy (DN).

miR-379KO mice were protected from early features of DN, and Fis1 expression was decreased in kidneys of diabetic WT but not diabetic miR-379KO mice. These results validate CLASH for identifying targets of miR-379, and identify Fis1 as a potential therapeutic target for DN.

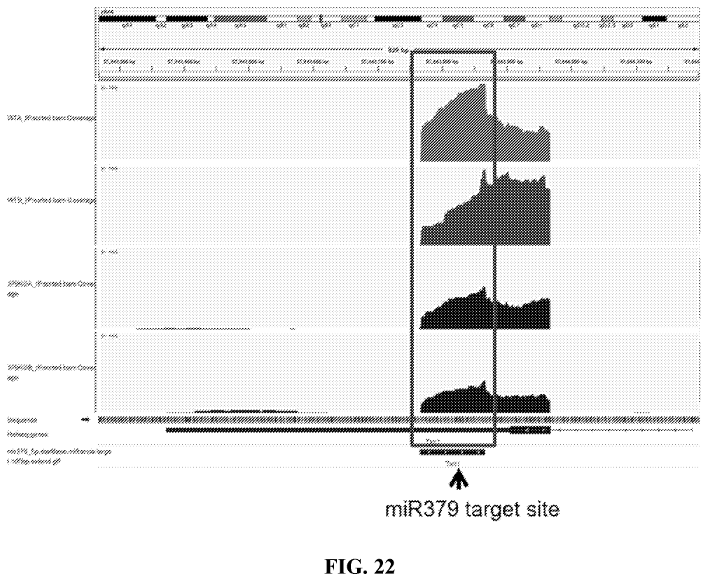

Similarly, Txn1 was confirmed as a miR-379 target by Ago2 qPCR and 3′UTR reporter assays. FIG. 22 depicts enrichment of RNA reads at 3′UTR of Txn1 gene and its significant reduction in miR-379KO MMC, suggesting Txn1 as a miR-379 target. Significant decrease of WT Txn1 3′UTR luciferase reporter by miR-379, compared with no change in mutant Txn1 3′UTR reporter by miR-379, also indicated Txn1 3′UTR is a true target of miR-379, as illustrated in FIG. 25B.

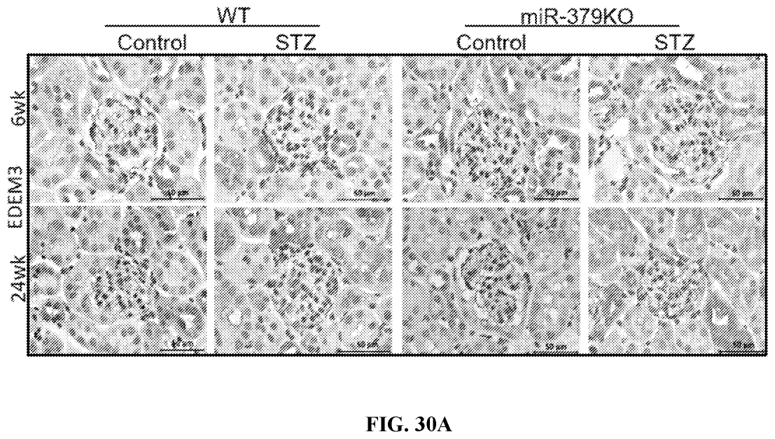

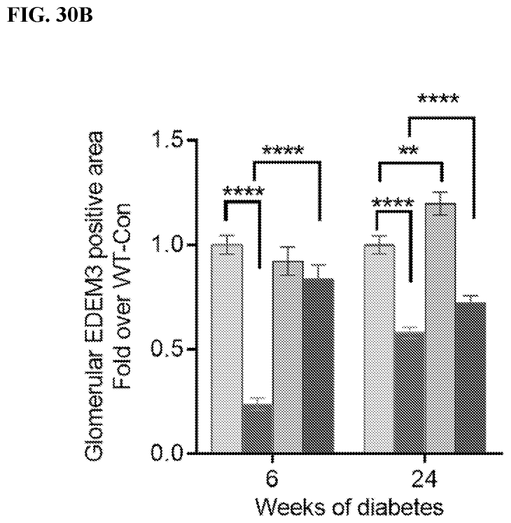

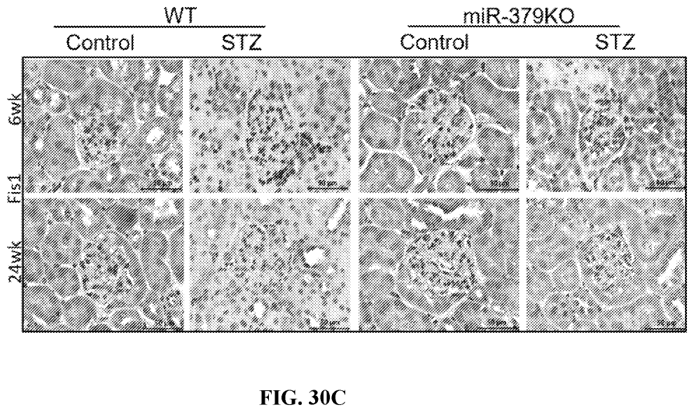

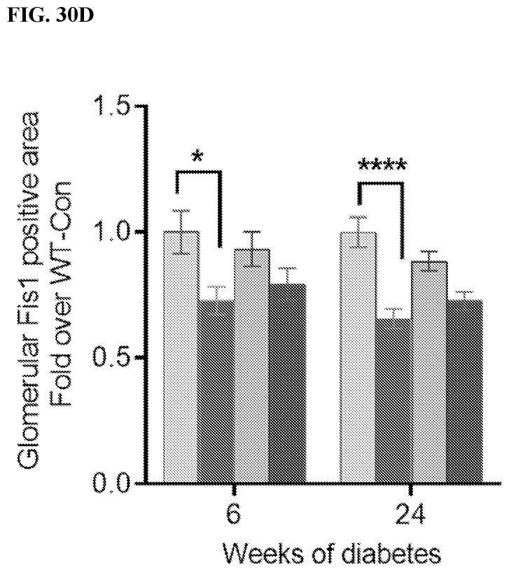

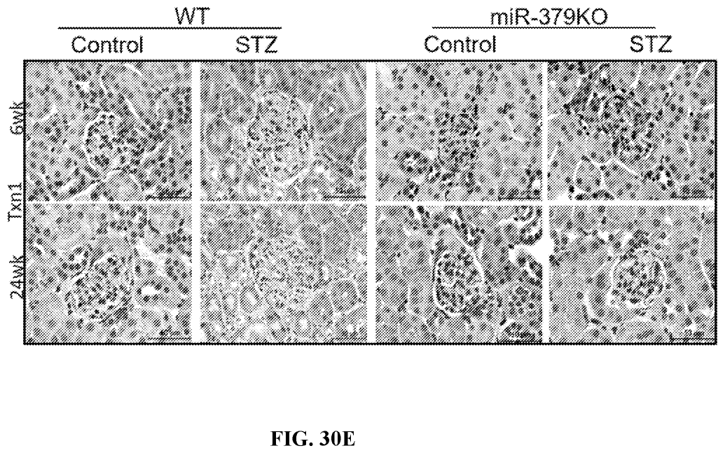

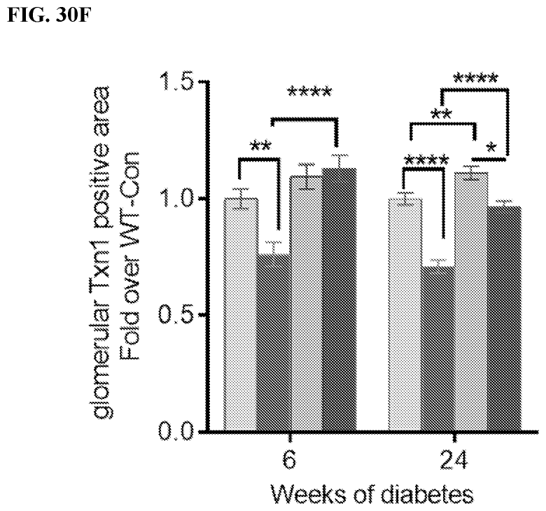

Further, presence of candidate miR-379 targets EDEM3, Fis1 and Txn1 were evaluated in WT and miR-379KO diabetic mice. As illustrated in FIGS. 30A-B, the EDEM3-positive glomerular area was significantly smaller in WT-STZ mice compared to non-diabetic controls; this decrease was restored in miR-379KO-STZ mice. FIGS. 30C-D illustrate that Fis1-positive glomerular area also showed a significant decrease in WT-STZ mice, which was reversed in miR-379KO-STZ mice. FIGS. 30E-F show Txn1 staining was weaker in both the cytoplasm and nucleus in WT-STZ mice compared to WT non-diabetic mice, but significantly higher in miR-379KO-STZ than WT-STZ mice. These results further validate these candidates as targets of miR-379, identify the candidates as potential therapeutic targets for DN, and further support the use of GapmeRs targeting lncMGC as therapeutic agents to treat diabetes, obesity, and related complications.

### Example 9: Effect of miR-379 on Mitochondrial Function

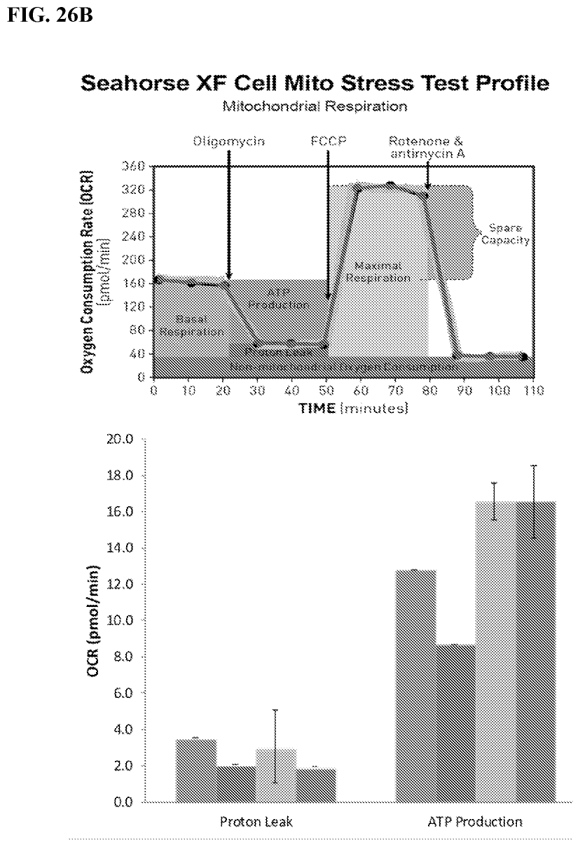

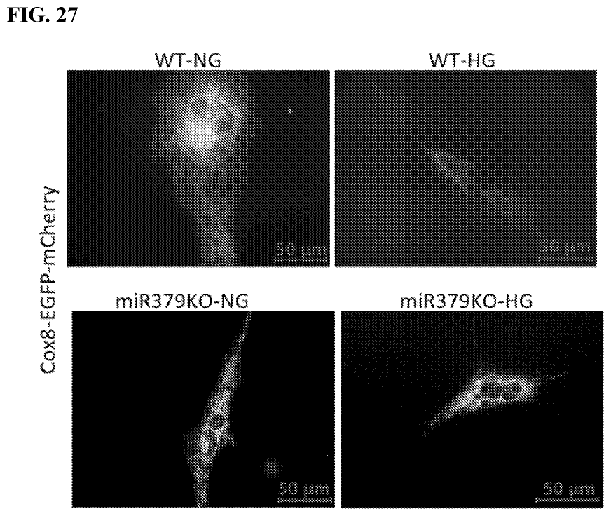

FIGS. 26A-B illustrate results of Seahorse XF Cell Mito Stress Tests for mitochondrial function to determine the effect of miR-379 on oxygen consumption rates (OCR). OCR were calculated in basal and spare respiratory capacity (SRC) levels in WT MMC and miR-379KO MMC. Significant reduction of mitochondrial activity in WT MMC subject to high glucose conditions (HG) was observed compared to WT MMC in low glucose conditions (LG). Mitochondrial activity was restored in miR-379KO MMC even after treatment with HG. To monitor mitochondrial quality and mitophagy, glomerular mesangial cells (from WT and miR-379KO mice) were transfected with pCLBW cox8 EGFP mCherry by Nucleofector (Amaxa Biosystems) using Basic Nucleofector Kit and the program U25. Three days after transfection, the cells were separated into two groups (normal glucose and high glucose). After 3 or 4 days of treatment with high glucose (or normal glucose), fluorescence (EGFP and mCherry) in live cells was detected by Keyence microscopy (Keyence). Results are illustrated in FIG. 27. Because EGFP is sensitive to acidic conditions, signals were decreased in damaged mitochondria of WT-HG cells, which have acidic internal conditions. High glucose conditions induced significant decrease in signal from EGFP in WT-HG MMC. This was not observed in miR-379KO-HG MMC, suggesting inhibition of miR-379 activity protects MMC from mitochondrial dysfunction.

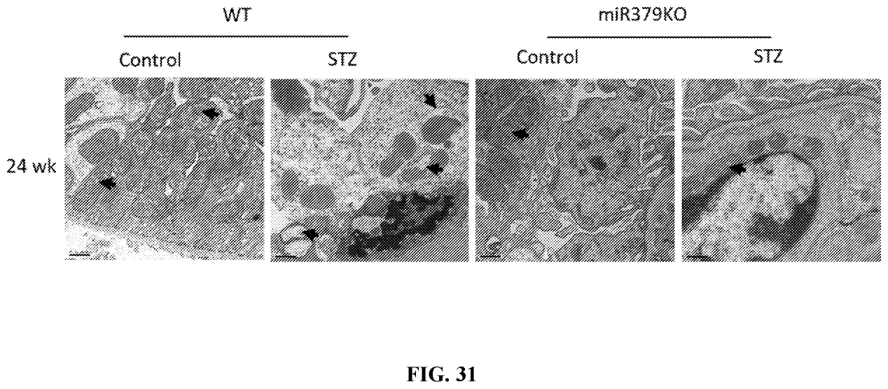

In vivo data further indicate miR-379 targeting protects mitochondria from damage in diabetes. FIG. 31 illustrates that miR-379KO mice show regular internal structure and elongated mitochondria 24 weeks after diabetes onset, while WT mice show disrupted cristae indicative of damaged mitochondrion. Together, these results illustrate protective effects conferred by miR-379 knockout, and indicate a therapeutic role for GapmeRs targeting lncMGC in the treatment of diabetes, obesity, and related complications.

### Example 10: Human lncMGC Interacting Proteins in Human Cells

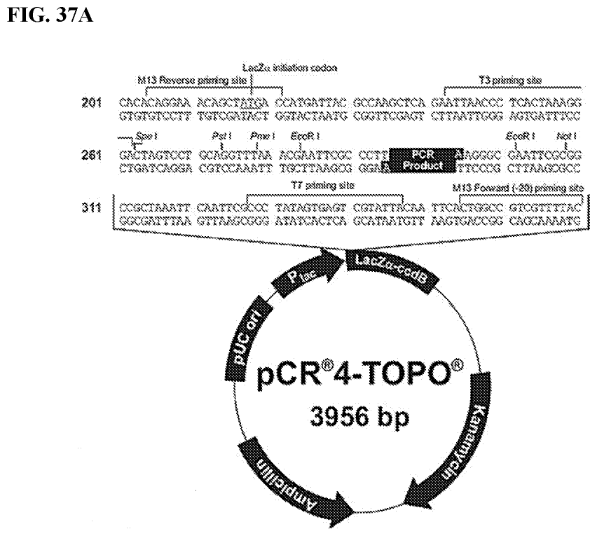

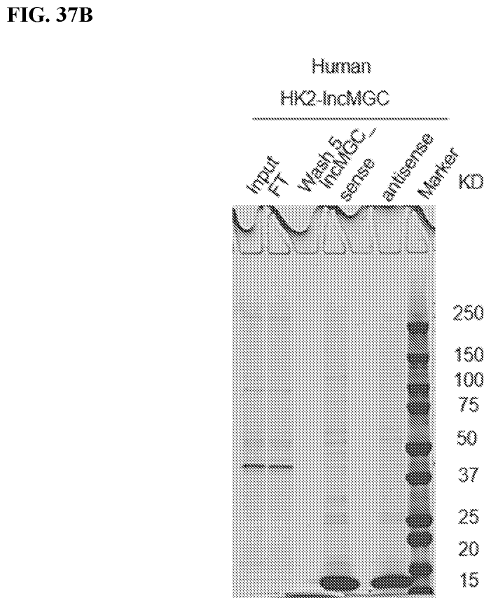

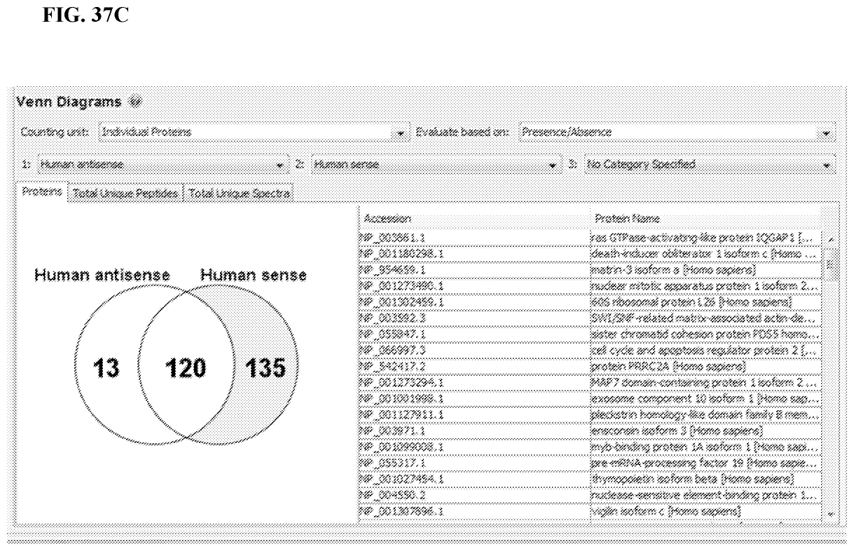

The experimental strategy for isolating and identifying proteins that interact with human lncMGC in human kidney cells (HK-2) is illustrated in FIGS. 37A-C. Proteins that interacted with either the sense or antisense strand of the human lncMGC sequence were separated by SDS-PAGE GEL. The isolated proteins were analyzed by mass-spectrometry, and 135 candidate human lncMGC interactive proteins were identified.

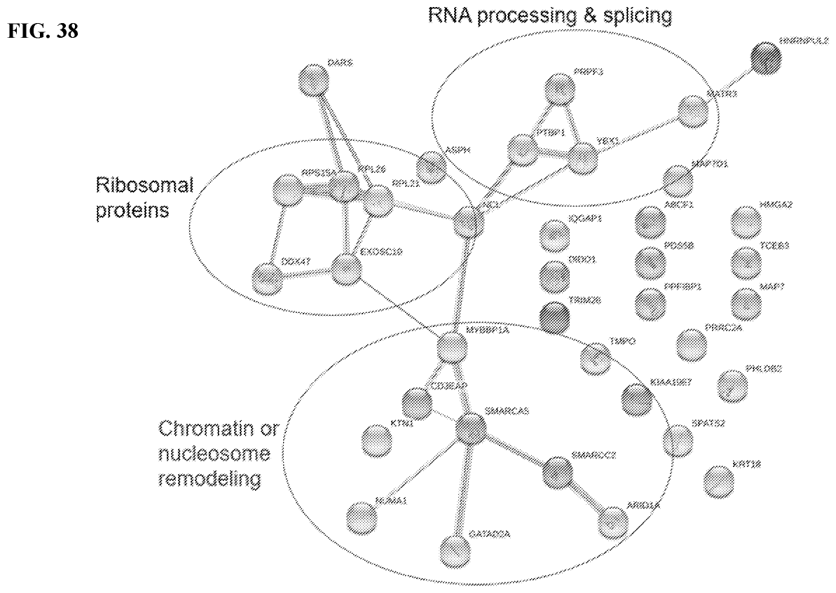

Among these candidates, ribosomal, RNA processing and splicing, and chromatin or nucleosome remodeling proteins were detected as shown in FIG. 38. These results suggest that human lncMGC may be involved in chromatin or nucleosome remodeling to regulate RNA. In embodiments, GapmeR targeting lncMGC reduces the expression of those RNAs (and treats human diseases) by inhibiting nucleosome remodeling.

### Example 11: Regeneration of Insulin-Secreting Cells by GapmeR MGC10

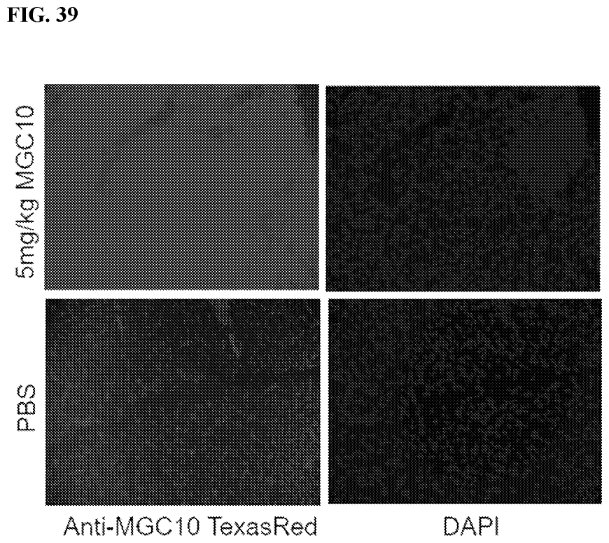

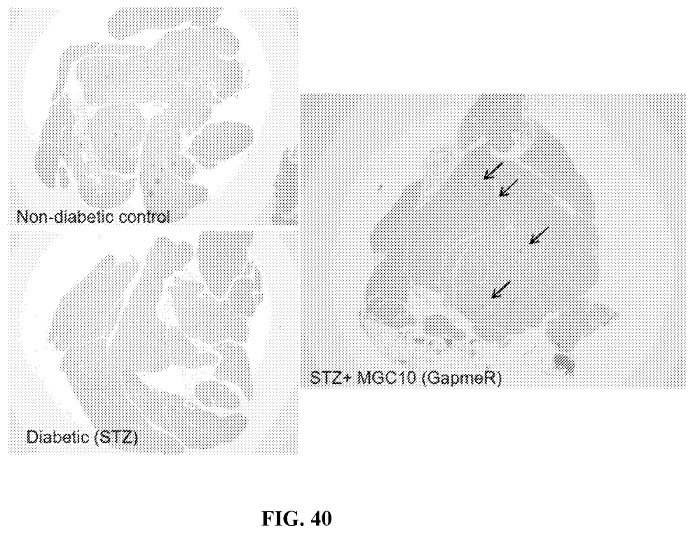

GapmeR MGC10 was evaluated for delivery and effectiveness in vivo. At 48 hours after injection of 5 mg/kg MGC10, GapmeR MGC10 was detected in mouse pancreas by in situ hybridization using anti-MGC10 TexasRed, as shown in FIG. 39. This result demonstrates efficient delivery of the GapmeR. To test the activity of MCG10 in vivo, mice were injected with STZ to induce diabetes. Illustrative images of pancreatic tissue from mice treated with MGC10 (STZ+MGC10), and control mice (Diabetic and Non-diabetic control) are shown in FIG. 40. Pancreatic tissue from the diabetic and non-diabetic control mice were stained for insulin-positive cells. No islets were detected in the pancreas of diabetic mice. However, diabetic mice injected with MGC10 showed insulin-positive islet cell clusters. These results indicate that targeting lncRNA-MGC with GapmeR MGC10 resulted in protection and/or regeneration of pancreatic islets.

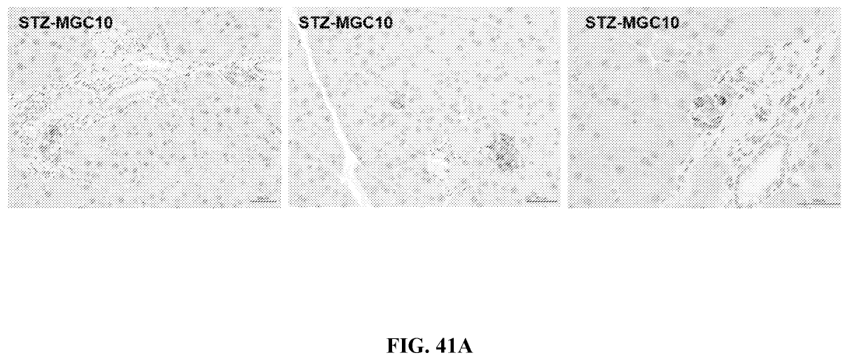

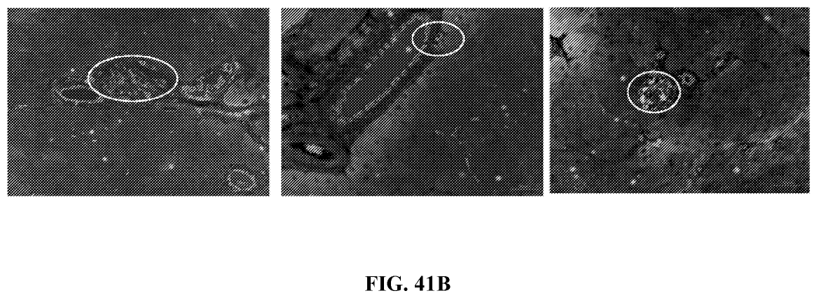

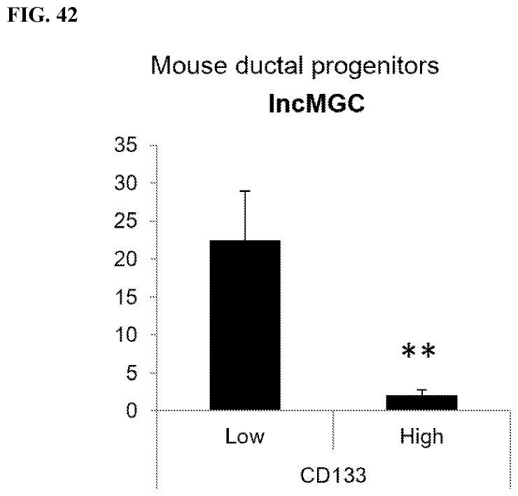

Further, in STZ-induced diabetic mice treated with GapmeR MGC10, insulin-positive cells were observed, as shown in representative images in FIG. 41A. The insulin-positive cells in STZ were close to duct cells as shown in FIG. 41B. These results suggest GapmeR MGC10 regenerates insulin-positive cells or stimulates trans-differentiation of islet cells from duct cells. As depicted in FIG. 42, mouse duct progenitor cells show statistically significantly lower expression of lncMGC than CD133. Cells with low levels of CD133 display higher expression of lncMGC than in mouse duct progenitor cells. These results further support inhibition of lncMGC by GapmeR MGC10 enhances regeneration of duct cells and trans-differentiation of duct cells to insulin-positive cells.

### Example 12: Human lncMGC is Increased in Human 1.1B4 β-cell line by Treatment with Cytokines and is Inhibited by GapmeR

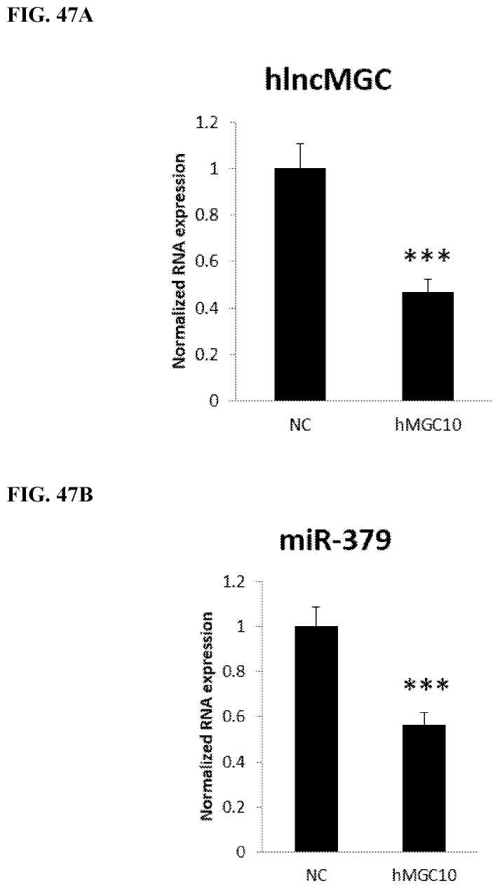

The effect of the cytokines TNF-α, INF-γ, and IL-1β on lncMGC levels in human 1.1B4 β-cells was evaluated. As illustrated in FIG. 46, treatment of 1.1B4 cells with the above cytokines increases levels of hlncMGC expression as compared to untreated cells. By contrast, human lncMGC and miR-379 expression was significantly reduced by treatment with 100 nM Gapmer HMGC10 for 4 days as shown in FIGS. 47A-B. These results indicate that HMGC10 is effective in reducing lncMGC levels, and may be capable of protecting β-cells from an immune response.

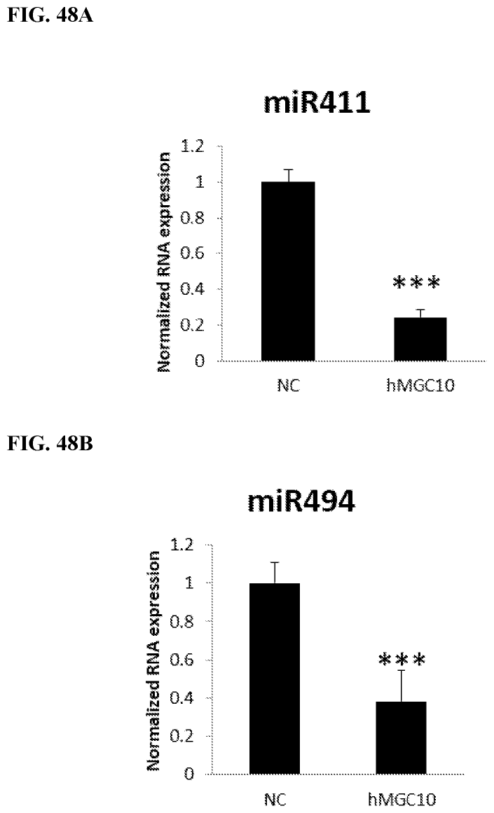

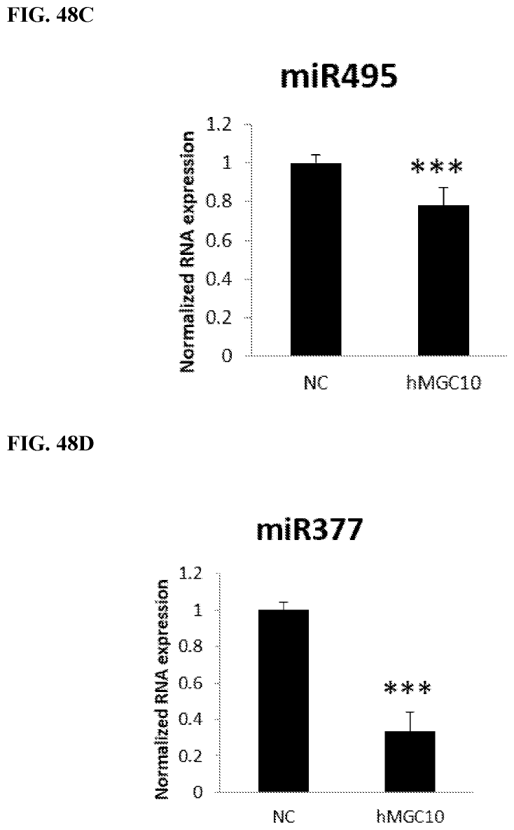

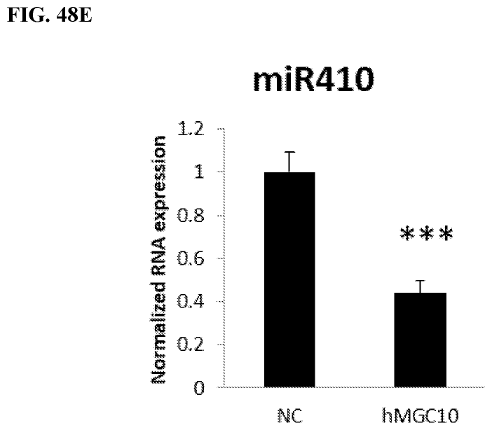

Human 1.1B4 cells were treated with GapmeR HMGC10 to evaluate the effect of humanized GapmeRs on expression of other miR-379 cluster miRNAs in human β-cells. As illustrated in FIGS. 48A-E, expression of members of the miR-379 cluster, including miR411, miR494, miR495, miR377, and miR410, were significantly reduced upon treatment GapmeR HMGC10, further illustrating the efficacy of GapmeRs in regulating multiple miRNAs in the cluster.

### Example 13: miR379 KO mice are Protected Against Type 1 Diabetes (T1D)-Induced Skeletal Muscle Atrophy

The miR379 knockout (KO) mice were evaluated for effects on diabetes-induced skeletal muscle atrophy. miR379 KO mice had significantly lower fasting blood glucose levels compared to WT mice, one week after diabetes development. Moreover, T1D-associated loss in body weight, muscle and fat mass was significantly ameliorated in miR379 KO mice relative to WT mice. Toward interrogating the underlying mechanism, the expression of atrophy-associated genes and miR379 target genes were assessed in skeletal muscle samples. In WT mice, T1D induced a loss of mixed muscle fiber such as Gastrocnemius (GAST), and this effect was underpinned by the increase in expression of muscle atrophy-associated genes such as Atrogin1 and myostatin, a negative regulator of muscle mass. Conversely, T1D induced an increase in miR379 and parallel decrease in miR379 target genes such as FIS1 and EDEM3 which are involved in regulation of mitochondrial health and ER stress respectively. miR379 KO mice also exhibited significant protection from these T1D-induced changes, supporting a role of miR379 in the deterioration of SkM mass under T1D insult. Reduced miR-379 expression is therefore protective against muscle-wasting under diabetic conditions.

### Embodiments

The present disclosure further provides the following embodiments:

Embodiment 1. A method of treating a condition of a subject, the method comprising administering to said subject an effective amount of a compound comprising a nucleic acid sequence capable of hybridizing to an RNA sequence 10 to 270 nucleobases downstream of the transcription start site of a mammalian microRNA-379 transcript, wherein (i) said nucleic acid sequence comprises a nucleobase analog or a modified internucleotide linkage, and (ii) said condition is diabetes or obesity.

Embodiment 2. The method of Embodiment 1, wherein said condition is diabetes.

Embodiment 3. The method of Embodiment 1 or 2, wherein said compound inhibits expression of a long non-coding RNA (lncMGC) comprising microRNA-376a, microRNA-299, microRNA-376c, microRNA-410, microRNA-494, microRNA-380-5p, microRNA-369-3p, microRNA-300, microRNA-541, microRNA-329, microRNA-381, microRNA-411, microRNA-134, microRNA-379, microRNA-154, microRNA-382, microRNA-376b, microRNA-496, microRNA-409-5p, microRNA-543, microRNA-377, microRNA-380-3p, or microRNA-495, in said subject.

Embodiment 4. The method of any one of Embodiments 1-3, wherein said compound inhibits expression of a microRNA-379 gene cluster.

Embodiment 5. The method of any one of Embodiments 1-4, wherein said nucleobase analog is at the 5′-end or the 3′-end of said nucleic acid sequence.

Embodiment 6. The method of any one of Embodiments 1-5, wherein said nucleic acid sequence comprises three nucleobase analogs at the 5′-end or the 3′-end of said nucleic acid sequence.

Embodiment 7. The method of any one of Embodiments 1-6, wherein said nucleobase analog is a Locked Nucleic Acid (LNA), 2′-O-alkyl nucleobase, 2′-Fluoro nucleobase, or 2′-OMe nucleobase

Embodiment 8. The method of any one of Embodiments 1-7, wherein said RNA sequence is 11 to 27, 61 to 93, 115 to 139, or 246 to 265 nucleobases downstream of said transcription start site

Embodiment 9. The method of any one of Embodiments 1-8, wherein said nucleic acid sequence comprises a modified internucleotide linkage

Embodiment 10. The method of Embodiment 9, wherein said modified internucleotide linkage is a phosphorothioate linkage

Embodiment 11. The method of any one of Embodiments 1-10, wherein said nucleic acid sequence has at least 90% sequence identity with a continuous 10 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130

Embodiment 12. The method of Embodiment 11, wherein said nucleic acid sequence has at least 90% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130

Embodiment 13. The method of any one of Embodiments 1-12, wherein said nucleic acid sequence is 10 to 30 nucleobases in length

Embodiment 14. Use of a compound for treating diabetes or obesity in a subject, wherein (i) said compound comprises a nucleic acid sequence capable of hybridizing to an RNA sequence 10 to 270 nucleobases downstream of the transcription start site of a mammalian microRNA-379 transcript, and (ii) said nucleic acid sequence comprises a nucleobase analog or a modified internucleotide linkage.

Embodiment 15. Use of a compound in the manufacture of a medicament for the treatment of diabetes or obesity in a subject, wherein (i) said compound comprises a nucleic acid sequence capable of hybridizing to an RNA sequence 10 to 270 nucleobases downstream of the transcription start site of a mammalian microRNA-379 transcript, and (ii) said nucleic acid sequence comprises a nucleobase analog or a modified internucleotide linkage.

Embodiment 16. A genetically engineered non-human animal comprising a recombinant nucleic acid molecule stably integrated into the genome of said animal, wherein (i) said recombinant nucleic acid molecule encodes an RNA sequence 10 to 270 nucleobases downstream of the transcription start site of a human microRNA-379 transcript, and (ii) said recombinant nucleic acid differs in sequence from a corresponding wild-type nucleic acid of non-human animals of the same type.

Embodiment 17. The transgenic non-human animal of Embodiment 16, wherein said non-human animal is a mouse.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure within known or customary practice within the art to which the disclosure pertains and may be applied to the essential features hereinbefore set forth, and follows in the scope of the appended claims.

What is claimed is:

1. A method of treating a condition of a subject, the method comprising administering to said subject an effective amount of a compound comprising a nucleic acid sequence capable of hybridizing to an RNA sequence 10 to 270 nucleobases downstream of the transcription start site of a mammalian microRNA-379 transcript, wherein (i) said nucleic acid sequence comprises a nucleobase analog or a modified internucleotide linkage, and (ii) said condition is diabetes or obesity.

2. The method of claim 1, wherein said condition is diabetes.

3. The method of claim 1 or 2, wherein said compound inhibits expression of a long non-coding RNA (lncMGC) comprising microRNA-379, microRNA-376a, microRNA-299, microRNA-376c, microRNA-410, microRNA-494, microRNA-380-5p, microRNA-369-3p, microRNA-300, microRNA-541, microRNA-329, microRNA-381, microRNA-411, microRNA-134, microRNA-154, microRNA-382, microRNA-376b, microRNA-496, microRNA-409-5p, microRNA-543, microRNA-377, microRNA-380-3p, or microRNA-495, in said subject.

4. The method of claim 1 or 2, wherein said compound inhibits expression of a microRNA-379 gene cluster.

5. The method of claim 1 or 2, wherein said nucleobase analog is at the 5′-end or the 3′-end of said nucleic acid sequence.

6. The method of claim 1 or 2, wherein said nucleic acid sequence comprises three nucleobase analogs at the 5′-end or the 3′-end of said nucleic acid sequence.

7. The method of claim 1 or 2, wherein said nucleobase analog is a Locked Nucleic Acid (LNA), 2′-O-alkyl nucleobase, 2′-Fluoro nucleobase, or 2′-OMe nucleobase.

8. The method of claim 1 or 2, wherein said RNA sequence is 11 to 27, 61 to 93, 115 to 139, or 246 to 265 nucleobases downstream of said transcription start site.

9. The method of claim 1 or 2, wherein said nucleic acid sequence comprises a modified internucleotide linkage.

10. The method of claim 9, wherein said modified internucleotide linkage is a phosphorothioate linkage.

11. The method of claim 1 or 2, wherein said nucleic acid sequence has at least 90% sequence identity with a continuous 10 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130.

12. The method of claim 1 or 2, wherein said nucleic acid sequence has at least 90% sequence identity with a continuous 11, 12, 13, 14, 15, 16, or 17 nucleobase sequence of SEQ ID NO: 1, 2, 3, 4, 5, 6, 7, 8, 9, 10, 11, 12, 13, 14, 15, 16, 17, 18, 19, 20, 21, 22, 23, 24, 128, 129, or 130.

13. The method of claim 1 or 2, wherein said nucleic acid sequence is 10 to 30 nucleobases in length.

14. Use of a compound for treating diabetes or obesity in a subject, wherein (i) said compound comprises a nucleic acid sequence capable of hybridizing to an RNA sequence 10 to 270 nucleobases downstream of the transcription start site of a mammalian microRNA-379 transcript, and (ii) said nucleic acid sequence comprises a nucleobase analog or a modified internucleotide linkage.

15. Use of a compound in the manufacture of a medicament for the treatment of diabetes or obesity in a subject, wherein (i) said compound comprises a nucleic acid sequence capable of hybridizing to an RNA sequence 10 to 270 nucleobases downstream of the transcription start site of a mammalian microRNA-379 transcript, and (ii) said nucleic acid sequence comprises a nucleobase analog or a modified internucleotide linkage.

16. A genetically engineered non-human animal comprising a recombinant nucleic acid molecule stably integrated into the genome of said animal, wherein (i) said recombinant nucleic acid molecule encodes an RNA sequence 10 to 270 nucleobases downstream of the transcription start site of a human microRNA-379 transcript, and (ii) said recombinant nucleic acid differs in sequence from a corresponding wild-type nucleic acid of non-human animals of the same type.

17. The transgenic non-human animal of claim 16, wherein said non-human animal is a mouse.
